SEQUENCE LISTING (28 sequences)

SEQ ID NO: 1 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 2 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 3 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 4 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 5 - DNA, 2198 bp, Cannabis sativa
  source 1..2198: mol_type = other DNA
cactctgtggtctcaaatgggcaagaactacaagagcctggattctgtggtggccagcgatttcattgctctgggtattacttctgaggtggccgagactcttcatggtagacttgctgagatcgtgtgcaactacggtgctgctactcctcagacctggatcaacattgctaaccatatcctgtctccggacctgcctttctcacttcaccagatgcttttctacggctgctacaaggatttcggtcctgctcctcctgcttggattcctgatcctgagaaggtgaagtctactaaccttggcgctctgcttgagaagcggggtaaagagtttctgggcgtcaagtacaaggacccgatcagctcattcagccacttccaagagttcagcgtgaggaaccctgaagtgtattggaggactgtgctgatggacgagatgaagatcagcttcagcaaggaccctgagtgcattctgagaagggacgacattaacaaccctggtggttctgaatggcttcctggtggctaccttaacagcgctaagaactgcctgaacgtgaacagcaacaagaaactgaacgacaccatgatcgtgtggcgtgatgagggtaacgatgatctgcctcttaacaagctgaccctggatcagctgaggaagagagtttggcttgttggctacgctctggaagagatgggtcttgaaaagggttgcgctatcgctatcgacatgcctatgcatgttgacgccgtggtgatctaccttgctattgtgcttgctggttacgtggtggtgtctatcgctgactctttcagcgctcctgagatttctaccaggctgaggctttctaaggccaaggctattttcacccaggaccacatcatcaggggcaagaagaggattcctctgtacagcagagtggtcgaggctaagtctcctatggctattgtgatcccttgcagcggttctaacattggggctgagcttagagatggggacatcagctgggactactttctcgagagggccaaagagttcaagaactgcgagttcactgctcgtgagcagcctgttgatgcttacaccaacatcctgttcagctctggtactaccggtgagcctaaggctattccttggactcaagctacccctcttaaggctgctgctgatggttggagccacctggatattagaaagggtgacgtgatcgtctggccgaccaatcttggttggatgatgggaccttggctggtgtacgcttctcttcttaacggtgcttctatcgccctgtacaacggttctcctcttgtgtctggtttcgccaagttcgtgcaggatgctaaggttaccatgcttggtgtggtgccatctatcgtgaggtcatggaagtccactaactgcgtgtcaggctacgattggagcactatccgttgcttctcatcctctggcgaggcttctaatgtggatgagtacctttggctgatgggccgtgctaattacaagcctgtgattgagatgtgcggtggcactgaaattggtggcgctttttctgctggatccttcttgcaagctcagagcctgtcctcattctcctcacagtgtatgggttgcaccctgtacatcctggataagaacggttacccgatgccgaagaacaagcctggtattggtgaactggctctgggtcctgttatgttcggagcttctaagaccctgctgaacggtaaccaccacgacgtttacttcaagggcatgcctaccttgaacggtgaggtgttgagaaggcacggtgatattttcgagctgaccagcaacggttactaccatgctcatggaagggctgacgataccatgaacatcggtggcatcaagatcagcagtatcgagattgagcgggtgtgcaatgaggtggacgatagggttttcgagactaccgctattggtgtgcctcctcttggtggtggtcctgagcaacttgtgattttcttcgtgctgaaggacagcaacgataccaccatcgatctgaaccagctccggctttctttcaaccttggtctgcagaagaagctgaacccgcttttcaaggtgacaagggttgtgcctcttagctctttgcctaggactgccaccaacaagattatgagaagggtgctgaggcagcagttctcccattttgaataggctttgagaccacgaagtg

SEQ ID NO: 6 - DNA, 2201 bp, Cannabis sativa
  source 1..2201: mol_type = other DNA
cactctgtggtctcaaggtatgggcaagaactacaagagcctggattctgtggtggccagcgatttcattgctctgggtattacttctgaggtggccgagactcttcatggtagacttgctgagatcgtgtgcaactacggtgctgctactcctcagacctggatcaacattgctaaccatatcctgtctccggacctgcctttctcacttcaccagatgcttttctacggctgctacaaggatttcggtcctgctcctcctgcttggattcctgatcctgagaaggtgaagtctactaaccttggcgctctgcttgagaagcggggtaaagagtttctgggcgtcaagtacaaggacccgatcagctcattcagccacttccaagagttcagcgtgaggaaccctgaagtgtattggaggactgtgctgatggacgagatgaagatcagcttcagcaaggaccctgagtgcattctgagaagggacgacattaacaaccctggtggttctgaatggcttcctggtggctaccttaacagcgctaagaactgcctgaacgtgaacagcaacaagaaactgaacgacaccatgatcgtgtggcgtgatgagggtaacgatgatctgcctcttaacaagctgaccctggatcagctgaggaagagagtttggcttgttggctacgctctggaagagatgggtcttgaaaagggttgcgctatcgctatcgacatgcctatgcatgttgacgccgtggtgatctaccttgctattgtgcttgctggttacgtggtggtgtctatcgctgactctttcagcgctcctgagatttctaccaggctgaggctttctaaggccaaggctattttcacccaggaccacatcatcaggggcaagaagaggattcctctgtacagcagagtggtcgaggctaagtctcctatggctattgtgatcccttgcagcggttctaacattggggctgagcttagagatggggacatcagctgggactactttctcgagagggccaaagagttcaagaactgcgagttcactgctcgtgagcagcctgttgatgcttacaccaacatcctgttcagctctggtactaccggtgagcctaaggctattccttggactcaagctacccctcttaaggctgctgctgatggttggagccacctggatattagaaagggtgacgtgatcgtctggccgaccaatcttggttggatgatgggaccttggctggtgtacgcttctcttcttaacggtgcttctatcgccctgtacaacggttctcctcttgtgtctggtttcgccaagttcgtgcaggatgctaaggttaccatgcttggtgtggtgccatctatcgtgaggtcatggaagtccactaactgcgtgtcaggctacgattggagcactatccgttgcttctcatcctctggcgaggcttctaatgtggatgagtacctttggctgatgggccgtgctaattacaagcctgtgattgagatgtgcggtggcactgaaattggtggcgctttttctgctggatccttcttgcaagctcagagcctgtcctcattctcctcacagtgtatgggttgcaccctgtacatcctggataagaacggttacccgatgccgaagaacaagcctggtattggtgaactggctctgggtcctgttatgttcggagcttctaagaccctgctgaacggtaaccaccacgacgtttacttcaagggcatgcctaccttgaacggtgaggtgttgagaaggcacggtgatattttcgagctgaccagcaacggttactaccatgctcatggaagggctgacgataccatgaacatcggtggcatcaagatcagcagtatcgagattgagcgggtgtgcaatgaggtggacgatagggttttcgagactaccgctattggtgtgcctcctcttggtggtggtcctgagcaacttgtgattttcttcgtgctgaaggacagcaacgataccaccatcgatctgaaccagctccggctttctttcaaccttggtctgcagaagaagctgaacccgcttttcaaggtgacaagggttgtgcctcttagctctttgcctaggactgccaccaacaagattatgagaagggtgctgaggcagcagttctcccattttgaataggctttgagaccacgaagtg

SEQ ID NO: 7 - DNA, 2198 bp, Cannabis sativa
  source 1..2198: mol_type = other DNA
cactctgtggtctcaaggtatgggcaagaactacaagagcctggattctgtggtggccagcgatttcattgctctgggtattacttctgaggtggccgagactcttcatggtagacttgctgagatcgtgtgcaactacggtgctgctactcctcagacctggatcaacattgctaaccatatcctgtctccggacctgcctttctcacttcaccagatgcttttctacggctgctacaaggatttcggtcctgctcctcctgcttggattcctgatcctgagaaggtgaagtctactaaccttggcgctctgcttgagaagcggggtaaagagtttctgggcgtcaagtacaaggacccgatcagctcattcagccacttccaagagttcagcgtgaggaaccctgaagtgtattggaggactgtgctgatggacgagatgaagatcagcttcagcaaggaccctgagtgcattctgagaagggacgacattaacaaccctggtggttctgaatggcttcctggtggctaccttaacagcgctaagaactgcctgaacgtgaacagcaacaagaaactgaacgacaccatgatcgtgtggcgtgatgagggtaacgatgatctgcctcttaacaagctgaccctggatcagctgaggaagagagtttggcttgttggctacgctctggaagagatgggtcttgaaaagggttgcgctatcgctatcgacatgcctatgcatgttgacgccgtggtgatctaccttgctattgtgcttgctggttacgtggtggtgtctatcgctgactctttcagcgctcctgagatttctaccaggctgaggctttctaaggccaaggctattttcacccaggaccacatcatcaggggcaagaagaggattcctctgtacagcagagtggtcgaggctaagtctcctatggctattgtgatcccttgcagcggttctaacattggggctgagcttagagatggggacatcagctgggactactttctcgagagggccaaagagttcaagaactgcgagttcactgctcgtgagcagcctgttgatgcttacaccaacatcctgttcagctctggtactaccggtgagcctaaggctattccttggactcaagctacccctcttaaggctgctgctgatggttggagccacctggatattagaaagggtgacgtgatcgtctggccgaccaatcttggttggatgatgggaccttggctggtgtacgcttctcttcttaacggtgcttctatcgccctgtacaacggttctcctcttgtgtctggtttcgccaagttcgtgcaggatgctaaggttaccatgcttggtgtggtgccatctatcgtgaggtcatggaagtccactaactgcgtgtcaggctacgattggagcactatccgttgcttctcatcctctggcgaggcttctaatgtggatgagtacctttggctgatgggccgtgctaattacaagcctgtgattgagatgtgcggtggcactgaaattggtggcgctttttctgctggatccttcttgcaagctcagagcctgtcctcattctcctcacagtgtatgggttgcaccctgtacatcctggataagaacggttacccgatgccgaagaacaagcctggtattggtgaactggctctgggtcctgttatgttcggagcttctaagaccctgctgaacggtaaccaccacgacgtttacttcaagggcatgcctaccttgaacggtgaggtgttgagaaggcacggtgatattttcgagctgaccagcaacggttactaccatgctcatggaagggctgacgataccatgaacatcggtggcatcaagatcagcagtatcgagattgagcgggtgtgcaatgaggtggacgatagggttttcgagactaccgctattggtgtgcctcctcttggtggtggtcctgagcaacttgtgattttcttcgtgctgaaggacagcaacgataccaccatcgatctgaaccagctccggctttctttcaaccttggtctgcagaagaagctgaacccgcttttcaaggtgacaagggttgtgcctcttagctctttgcctaggactgccaccaacaagattatgagaagggtgctgaggcagcagttctcccattttgaaggtgtgagaccacgaagtg

SEQ ID NO: 8 - DNA, 1193 bp, Cannabis sativa
  source 1..1193: mol_type = other DNA
cactctgtggtctcaaatgaaccaccttagagctgagggtcctgcttctgtgcttgctattggtactgctaaccccgagaacattctgctgcaggatgagttccctgactactacttcagggtgaccaagtctgagcacatgacccagctgaaagagaagttccggaagatctgcgacaagagcatgatccggaagaggaactgcttcctgaacgaggaacacctgaagcagaatcctaggcttgttgagcatgagatgcagacccttgatgctaggcaggatatgcttgttgttgaggtgccaaagctcggcaaggatgcttgtgctaaggctatcaaagaatggggccagccgaagtctaagatcacccaccttattttcaccagcgccagcactactgatatgcctggtgctgattaccactgcgctaagctgcttggtcttagcccttctgttaagcgggtgatgatgtaccagcttggttgctatggtggtggaaccgtgcttaggatcgctaaggatatcgccgagaacaacaagggtgctagagttcttgctgtgtgctgcgatattatggcctgcctttttaggggccctagcgagtctgatcttgagcttcttgttggccaggctatcttcggtgatggtgctgctgctgttattgtgggtgctgaacctgatgagtctgttggtgagaggcctattttcgagcttgtgtctaccggtcagaccatccttcctaactctgagggtactatcggtggccacattagagaggctggccttatcttcgatctgcacaaggatgtgccgatgctgatctccaacaacatcgagaagtgcctgatcgaggctttcacccctatcggtatcagcgactggaacagcattttctggatcactcaccctggtggcaaggctattctggataaggtggaagagaagctgcacctgaagtccgataagttcgtggactctaggcacgtgttgtcagagcacggtaacatgtcatctagcaccgtgctgttcgtgatggatgagctgaggaagcgttctcttgaagagggaaagtctaccaccggtgatggatttgagtggggtgtgctttttggtttcggtcctggtcttaccgttgagcgtgttgtggttagatccgtgccgatcaagtactaggctttgagaccacgaagtg

SEQ ID NO: 9 - DNA, 1196 bp, Cannabis sativa
  source 1..1196: mol_type = other DNA
cactctgtggtctcaaggtatgaaccaccttagagctgagggtcctgcttctgtgcttgctattggtactgctaaccccgagaacattctgctgcaggatgagttccctgactactacttcagggtgaccaagtctgagcacatgacccagctgaaagagaagttccggaagatctgcgacaagagcatgatccggaagaggaactgcttcctgaacgaggaacacctgaagcagaatcctaggcttgttgagcatgagatgcagacccttgatgctaggcaggatatgcttgttgttgaggtgccaaagctcggcaaggatgcttgtgctaaggctatcaaagaatggggccagccgaagtctaagatcacccaccttattttcaccagcgccagcactactgatatgcctggtgctgattaccactgcgctaagctgcttggtcttagcccttctgttaagcgggtgatgatgtaccagcttggttgctatggtggtggaaccgtgcttaggatcgctaaggatatcgccgagaacaacaagggtgctagagttcttgctgtgtgctgcgatattatggcctgcctttttaggggccctagcgagtctgatcttgagcttcttgttggccaggctatcttcggtgatggtgctgctgctgttattgtgggtgctgaacctgatgagtctgttggtgagaggcctattttcgagcttgtgtctaccggtcagaccatccttcctaactctgagggtactatcggtggccacattagagaggctggccttatcttcgatctgcacaaggatgtgccgatgctgatctccaacaacatcgagaagtgcctgatcgaggctttcacccctatcggtatcagcgactggaacagcattttctggatcactcaccctggtggcaaggctattctggataaggtggaagagaagctgcacctgaagtccgataagttcgtggactctaggcacgtgttgtcagagcacggtaacatgtcatctagcaccgtgctgttcgtgatggatgagctgaggaagcgttctcttgaagagggaaagtctaccaccggtgatggatttgagtggggtgtgctttttggtttcggtcctggtcttaccgttgagcgtgttgtggttagatccgtgccgatcaagtactaggctttgagaccacgaagtg

SEQ ID NO: 10 - DNA, 1193 bp, Cannabis sativa
  source 1..1193: mol_type = other DNA
cactctgtggtctcaaggtatgaaccaccttagagctgagggtcctgcttctgtgcttgctattggtactgctaaccccgagaacattctgctgcaggatgagttccctgactactacttcagggtgaccaagtctgagcacatgacccagctgaaagagaagttccggaagatctgcgacaagagcatgatccggaagaggaactgcttcctgaacgaggaacacctgaagcagaatcctaggcttgttgagcatgagatgcagacccttgatgctaggcaggatatgcttgttgttgaggtgccaaagctcggcaaggatgcttgtgctaaggctatcaaagaatggggccagccgaagtctaagatcacccaccttattttcaccagcgccagcactactgatatgcctggtgctgattaccactgcgctaagctgcttggtcttagcccttctgttaagcgggtgatgatgtaccagcttggttgctatggtggtggaaccgtgcttaggatcgctaaggatatcgccgagaacaacaagggtgctagagttcttgctgtgtgctgcgatattatggcctgcctttttaggggccctagcgagtctgatcttgagcttcttgttggccaggctatcttcggtgatggtgctgctgctgttattgtgggtgctgaacctgatgagtctgttggtgagaggcctattttcgagcttgtgtctaccggtcagaccatccttcctaactctgagggtactatcggtggccacattagagaggctggccttatcttcgatctgcacaaggatgtgccgatgctgatctccaacaacatcgagaagtgcctgatcgaggctttcacccctatcggtatcagcgactggaacagcattttctggatcactcaccctggtggcaaggctattctggataaggtggaagagaagctgcacctgaagtccgataagttcgtggactctaggcacgtgttgtcagagcacggtaacatgtcatctagcaccgtgctgttcgtgatggatgagctgaggaagcgttctcttgaagagggaaagtctaccaccggtgatggatttgagtggggtgtgctttttggtttcggtcctggtcttaccgttgagcgtgttgtggttagatccgtgccgatcaagtacggtgtgagaccacgaagtg

SEQ ID NO: 11 - DNA, 341 bp, Cannabis sativa
  source 1..341: mol_type = other DNA
cactctgtggtctcaaatggctgtgaagcaccttatcgtgctgaagttcaaggacgagattaccgaggctcagaaagaagagttcttcaagacctacgtgaacctggtgaacatcatcccggctatgaaggatgtgtactggggcaaagatgtgacccagaagaacaaagaagagggctacacccatatcgtcgaggtgacatttgagagcgtggaaaccatccaggactacatcattcaccctgctcacgttggtttcggcgacgtgtacagatctttctgggagaagctgctgatcttcgactacacccctaggaagtaggctttgagaccacgaagtg

SEQ ID NO: 12 - DNA, 344 bp, Cannabis sativa
  source 1..344: mol_type = other DNA
cactctgtggtctcaaggtatggctgtgaagcaccttatcgtgctgaagttcaaggacgagattaccgaggctcagaaagaagagttcttcaagacctacgtgaacctggtgaacatcatcccggctatgaaggatgtgtactggggcaaagatgtgacccagaagaacaaagaagagggctacacccatatcgtcgaggtgacatttgagagcgtggaaaccatccaggactacatcattcaccctgctcacgttggtttcggcgacgtgtacagatctttctgggagaagctgctgatcttcgactacacccctaggaagtaggctttgagaccacgaagtg

SEQ ID NO: 13 - DNA, 341 bp, Cannabis sativa
  source 1..341: mol_type = other DNA
cactctgtggtctcaaggtatggctgtgaagcaccttatcgtgctgaagttcaaggacgagattaccgaggctcagaaagaagagttcttcaagacctacgtgaacctggtgaacatcatcccggctatgaaggatgtgtactggggcaaagatgtgacccagaagaacaaagaagagggctacacccatatcgtcgaggtgacatttgagagcgtggaaaccatccaggactacatcattcaccctgctcacgttggtttcggcgacgtgtacagatctttctgggagaagctgctgatcttcgactacacccctaggaagggtgtgagaccacgaagtg

SEQ ID NO: 14 - DNA, 1232 bp, Cannabis sativa
  source 1..1232: mol_type = other DNA
cactctgtggtctcaaatgggcctttctctggtgtgcactttctcattccagaccaactaccacaccttgctgaacccgcataacaagaacccgaagaacagcctgctgagctaccagcatcctaagactccgatcatcaagtccagctacgacaacttcccgagcaagtactgcctgaccaagaacttccaccttctgggtctgaacagccacaacaggatcagctctcagagccggtctattagagctggtagcgatcagattgagggctctcctcatcacgagagcgataactctattgccaccaagatcctgaacttcggccatacctgttggaagctgcagaggccttatgtggtgaagggcatgatttctatcgcttgcggtctgttcggtcgtgagcttttcaacaacaggcacctgttctcttggggcttgatgtggaaggctttcttcgctcttgtgccgatcctgagcttcaacttcttcgccgctatcatgaaccagatctacgacgtggacatcgaccggatcaacaagcctgatcttcctttggtgtccggcgagatgtctattgagactgcttggatcctgtccatcatcgtggctcttaccggtcttatcgtgaccatcaagctgaagtctgctccgctgttcgtgttcatctacatcttcggtatcttcgccggcttcgcttactctgttcctcctattaggtggaagcagtacccgtttaccaacttcctgatcaccatctcctctcacgtgggtcttgctttcacctcttactctgctactaccagcgctcttggtctgccttttgtttggaggcctgccttcagcttcattatcgctttcatgaccgtgatgggcatgaccattgctttcgccaaggacatctctgatatcgagggtgacgctaagtacggtgtgtctactgtggctactaagcttggcgctaggaacatgactttcgtggtgtctggtgtgttgctgctgaactacctggtgagcatcagcatcggtatcatctggcctcaggtgttcaagagcaacatcatgatcctgtctcacgccatccttgctttctgccttatcttccagactcgtgagctggctcttgctaactatgcttctgctccttccaggcagtttttcgagttcatctggctgctgtactacgccgagtacttcgtgtacgtgttcatttgagctttgagaccacgaagtg

SEQ ID NO: 15 - DNA, 211 bp, Arabidopsis thaliana
  source 1..211: mol_type = other DNA
cactctgtggtctcaaatggcttcttctatgctttcttctgctgctgttgttgctactcgtgctagtgctgctcaagctagtatggttgctccttttactggacttaagtctgctgcttcttttcctgttactagaaagcaaaacaaccttgatattacttctattgctagtaacggaggaagagttcgagcaggttgagaccacgaagtg

SEQ ID NO: 16 - DNA, 757 bp, synthetic construct
  source 1..757: mol_type = other DNA
cactctgtggtctcaggtggcggctctatggtgtctaagggcgaagaggacaacatggcttctttgcctgctactcacgagctgcacatcttcggttctatcaacggtgtggacttcgacatggttggtcaaggtactggcaaccctaacgatggttacgaggaactgaacctgaagtccaccaagggtgatctgcagttctctccttggattctggtgcctcacatcggttacggtttccaccagtacctgccttatcctgatggcatgtctccattccaggctgctatggttgatggctctggttatcaggtgcacaggactatgcagttcgaggatggtgcttctctgaccgtgaattacaggtacacctacgagggctctcacatcaagggtgaagctcaggtgaaaggcactggttttccagctgatggtcctgtgatgaccaactctcttactgctgctgattggtgccggtccaagaaaacttacccaaacgacaagaccatcatcagcaccttcaagtggtcttacaccaccggtaacggtaagaggtacaggtctactgctaggaccacttacaccttcgctaagcctatggctgccaactaccttaagaaccagccgatgtacgtgttccgtaagaccgagcttaagcacagcaagaccgagttgaacttcaaagagtggcaaaaggccttcaccgacgtgatgggtatggatgagctttacaagtaggctttgagaccacgaagtg

SEQ ID NO: 17 - DNA, 332 bp, synthetic construct
  source 1..332: mol_type = other DNA
cactctgtggtctcaggaggttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgtacttgagaccacgaagtg

SEQ ID NO: 18 - DNA, 877 bp, synthetic construct
  source 1..877: mol_type = other DNA
cactctgtggtctcatactgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgtgagaccacgaagtg

SEQ ID NO: 19 - DNA, 1779 bp, synthetic construct
  source 1..1779: mol_type = other DNA
cactctgtggtctcagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagataatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtcctggttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggagaagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgtggttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgcgcttgagaccacgaagtg

SEQ ID NO: 20 - protein, 192 aa, Arabidopsis thaliana
MIRVTGTAAPAMSVVFPTSWRQPVMLPLRSAKTFKPHTFLDLKGGKEYSTKMSEFHEVELKVRDYELDQFGVVNNAVYANYCQHGMHEFLESIGINCDEVARSGEALAISELTMNFLAPLRSGDKFVVKVNISRTSAARIYFDHSILKLPNQEVILEAKATVVWLDNKHRPVRIPSSIRSKFVHFLRQNDTV

SEQ ID NO: 21 - protein, 188 aa, Arabidopsis thaliana
MIRVTGTAAPAMSVVFPTSWRQPVMLPLRSAKTFKPHTFLDLKGGKEMSEFHEVELKVRDYELDQFGVVNNAVYANYCQHGMHEFLESIGINCDEVARSGEALAISELTMNFLAPLRSGDKFVVKVNISRTSAARIYFDHSILKLPNQEVILEAKATVVWLDNKHRPVRIPSSIRSKFVHFLRQNDTV

SEQ ID NO: 22 - protein, 649 aa, Arabidopsis thaliana
MAFNTAMASTSPAAANDVLREHIGLRRSLSGQDLVLKGGGIRRSSSDNHLCCRSGNNNNRILAVSVRPGMKTSRSVGVFSFQISSSIIPSPIKTLLFETDTSQDEQESDEIEIETEPNLDGAKKANWVERLLEIRRQWKREQKTESGNSDVAEESVDVTCGCEEEEGCIANYGSVNGDWGRESFSRLLVKVSWSEAKKLSQLAYLCNLAYTIPEIKGEDLRRNYGLKFVTSSLEKKAKAAILREKLEQDPTHVPVITSPDLESEKQSQRSASSSASAYKIAASAASYIHSCKEYDLSEPIYKSAAAAQAAASTMTAVVAAGEEEKLEAARELQSLQSSPCEWFVCDDPNTYTRCFVIQGSDSLASWKANLFFEPTKFEDTDVLVHRGIYEAAKGIYEQFLPEITEHLSRHGDRAKFQFTGHSLGGSLSLIVNLMLISRGLVSSEAMKSVVTFGSPFVFCGGEKILAELGLDESHVHCVMMHRDIVPRAFSCNYPDHVALVLKRLNGSFRTHPCLNKNKLLYSPMGKVYILQPSESVSPTHPWLPPGNALYILENSNEGYSPTALRAFLNRPHPLETLSQRAAYGSEGSVLRDHDSKNYVKAVNGVLRQHTKLIVRKARIQRRSVWPVLTSAGRGLNESLTTAEEIMTRV

SEQ ID NO: 23 - protein, 896 aa, Solanum lycopersicum
MLKPQFQQSTKTLIPSWNTNTLFLASFPINILNKNFILKKKNNFRVHHNYNGANTIKAVLNSTQKSIGVKAVVTVQKQVNLNLLRGLDGIGDLLGKSLILWIVAAELDHKTGLEKPSIRSYAHRGLDVDGDTYYEADFEIPEDFGEVGAILVENEHHKEMYVKNIVIDGFVHAKVEITCNSWVHSKFANPDKRIFFTNKSYLPSQTPSGVIRLREGRTRTLRGDGVGERKVFERIYDYDVYNDLGEVVSNNDDAKRPILGGKKLPYPRRCRTGRQRSKKDPLYETRSTFVYVPRDEAFSAVKSLTFSGNTVYSALHAVVPALESVVSDPDLGFPHFPAIDSLFNVGVDLSGLSDKKSSLFNIVPRLIKSISETGKDVLLFESPQLVQRDKFSWFRDVEFARQTLAGLNPYSIRLVTEWPLRSNLDPKVSGPPESEITKELIENEIGNNMTVEQAVQQKKLFILDYHDLLLPYVNKVNELKGSVLYGSRTIFFLTPHGTLKPLAIELTRPPIDDKPQWKEVYSPNNWNATGAWLWKLAKAHVLSHDSGYHQLVSHWLRTHCCTEPYIIATNRQLSAMHPIYRLLHPHFRYTMEINALAREALINANGIIESSFFPGKYSVELSSIAYGAEWRFDQEALPQNLISRGLAEEDPNEPHGLKLAIEDYPFANDGLVLWDILKQWVTNYVNHYYPQTNLIESDKELQAWWSEIKNVGHGDKKDEPWWPELKTPNDLIGIITTIVWVTSGHHAAVNFGQYSYGGYFPNRPTTARSKMPTEDPTAEEWEWFLNKPEEALLRCFPSQIQATKVMTILDVLSNHSPDEEYIGEKIEPYWAEDPVINAAFEVFSGKLKELEGIIDARNNDSKLNNRNGAGVMPYELLKPYSEPGVTGKGVPYSISI

SEQ ID NO: 24 - DNA, 614 bp, Arabidopsis thaliana
  source 1..614: mol_type = other DNA
cactctgtggtctcaaatgatcagagtgaccggtactgctgctcctgctatgtctgttgtgttccctacttcttggaggcagcctgtgatgcttcctttgaggtctgctaagaccttcaagccgcacaccttccttgatctgaaaggcggcaaagaatactccaccaagatgagcgagttccacgaggttgagcttaaggtgagggattacgagctggatcagttcggtgtggtgaacaatgctgtgtacgctaactactgccagcacggtatgcatgagttccttgagagcatcggtatcaactgcgacgaggttgcaagatctggtgaggctcttgctattagcgagctgactatgaacttcctggctcctcttaggtccggcgataagttcgttgtgaaagtgaacatcagccggacctctgctgctaggatctacttcgatcacagcatcctgaagctgccgaatcaagaggttatcctcgaggctaaggctaccgttgtgtggcttgataacaagcacaggccagtgaggatcccgtcctctattaggtctaagttcgtgcacttcctgcggcagaatgatactgtgtaagctttgagaccacgaagtg

SEQ ID NO: 25 - DNA, 602 bp, Arabidopsis thaliana
  source 1..602: mol_type = other DNA
cactctgtggtctcaaatgatcagagtgaccggtactgctgctcctgctatgtctgttgtgttccctacttcttggaggcagcctgtgatgcttcctttgaggtctgctaagaccttcaagccgcacaccttccttgatctgaaaggcggcaaagaaatgagcgagttccacgaggttgagcttaaggtgagggattacgagctggatcagttcggtgtggtgaacaatgctgtgtacgctaactactgccagcacggtatgcatgagttccttgagagcatcggtatcaactgcgacgaggttgcaagatctggtgaggctcttgctattagcgagctgactatgaacttcctggctcctcttaggtccggcgataagttcgttgtgaaagtgaacatcagccggacctctgctgctaggatctacttcgatcacagcatcctgaagctgccgaatcaagaggttatcctcgaggctaaggctaccgttgtgtggcttgataacaagcacaggccagtgaggatcccgtcctctattaggtctaagttcgtgcacttcctgcggcagaatgatactgtgtaagctttgagaccacgaagtg

SEQ ID NO: 26 - DNA, 1985 bp, Arabidopsis thaliana
  source 1..1985: mol_type = other DNA
cactctgtggtctcaaatggctttcaacaccgctatggcttctacttctcctgctgctgctaacgatgtgctgagagagcacattggtcttaggcggtctttgtctggtcaggaccttgtgcttaaaggcggcggtattcgtaggtcctcttctgataatcacctgtgctgcaggtccgggaacaacaacaataggattctggctgtgtctgtgcggcctggtatgaaaacttctagatctgtgggcgtgttcagcttccagatctccagctctattatcccgagtccgattaagaccctgctgttcgagactgataccagccaggatgaacaagagtccgacgagattgagatcgagacagagcctaacctggatggcgctaagaaggctaattgggttgagcggcttcttgagattaggcgtcagtggaagagagagcaaaagaccgagagcggtaactctgatgtggctgaagagtctgtggatgtgacttgtggttgcgaagaggaagagggctgcattgctaattacggtagcgtgaacggtgattggggccgtgagtctttttctaggcttctggttaaggtgagctggtccgaggctaagaagctttctcagcttgcttacctgtgcaacctggcttacaccattcctgagatcaagggcgaagatctgcggaggaattacggtcttaagttcgtgaccagcagcctcgagaagaaagctaaggctgctatcctgcgtgagaagcttgaacaggatcctactcacgtgccagtgatcacctctcctgatcttgaaagcgagaagcagtctcagcggagcgcttctagttctgctagcgcttataagatcgctgcttccgctgctagctacatccactcttgcaaagagtacgatctgagcgagccgatctacaagtctgctgcagctgctcaagctgctgcttctactatgactgctgttgttgctgctggcgaggaagaaaagcttgaagctgctagagagctgcaaagcctgcaatcttctccatgcgagtggttcgtgtgcgacgatcctaatacttacacccggtgcttcgtgatccagggctctgattctcttgctagctggaaggctaacctgttcttcgagcctaccaagttcgaggacactgatgtgcttgttcacaggggaatctacgaggctgcaaagggaatctatgagcagttcctgcctgagattaccgagcacctttctaggcatggtgacagggctaagttccagttcaccggtcattctcttggcggtagcctttctcttatcgtgaacctgatgctgatctccaggggccttgtttcttcagaggctatgaagtctgtggtgaccttcggttctcctttcgttttctgtggtggcgagaagatccttgctgagcttggtcttgatgagtctcatgtgcactgcgtgatgatgcacagggatattgtgcctagggccttctcttgcaactaccctgatcatgtggctctggtgcttaagaggctgaacggttctttcaggactcacccttgcctgaacaagaacaagctcctgtacagccctatgggcaaagtgtacattctgcagcctagcgagtctgtgtctcctactcatccttggttgcctccaggtaacgctctgtacatcctcgagaattctaacgagggctactctcctactgctctgagggcttttcttaacaggcctcatcctctcgagactctgtctcaaagggctgcttatggttctgagggttctgtgcttagggaccacgactctaagaactacgtgaaggctgtgaatggtgtgttgaggcagcacaccaagctgattgtgaggaaggctaggattcagaggcgttctgtttggcctgtgcttacttctgctggtaggggtcttaacgagtctcttactaccgccgaggaaatcatgacccgggtttaggctttgagaccacgaagtg

SEQ ID NO: 27 - DNA, 2726 bp, Solanum lycopersicum
  source 1..2726: mol_type = other DNA
cactctgtggtctcaaatgctgaagcctcagttccagcagtctaccaagactctgatcccgagctggaacaccaataccttgttcctcgctagcttcccgatcaacatcctgaacaagaacttcattctgaagaagaagaacaacttccgggtccaccacaactacaacggtgctaacactatcaaggccgtgctgaacagcacccagaagtctattggagttaaggctgtggtgaccgtgcagaaacaggtgaaccttaaccttctgaggggccttgatggtatcggtgatctgcttggtaagagcctgattctgtggattgtggctgctgagcttgatcacaagaccggtttggagaagccgagcattaggtcttatgctcacaggggtcttgatgtggatggcgatacttactacgaggccgatttcgagatccctgaggattttggtgaggtgggcgctattcttgttgagaacgagcaccacaaagagatgtacgtcaagaacatcgtgatcgacggtttcgtgcacgccaaggttgagattacttgcaactcttgggtgcacagcaagttcgctaaccctgacaagaggatcttctttaccaacaagagctacctgccttctcagaccccttctggtgtgattagactgagagagggtagaaccaggaccttgagaggtgatggtgttggtgagaggaaggtgttcgagaggatctacgattacgacgtgtacaacgatcttggcgaggtggtgagcaacaacgatgatgctaagaggcctatccttggcggtaagaagctgccttatcctagaagatgcaggactggtaggcagcggtctaaaaaggatcctctgtacgagactcggagcaccttcgtttatgtgcctagagatgaggctttcagcgccgtgaagtctcttaccttctctggtaacaccgtgtactctgctctgcatgctgttgtgcctgctcttgagtctgttgtgtctgatcctgatctgggcttccctcacttccctgctattgactctcttttcaacgtgggcgttgacctgtctggcctgtctgataagaagtccagcctgttcaacatcgtgccgaggctgatcaagagcatctctgagactggtaaggacgtgctgcttttcgagtctcctcagcttgttcagcgggacaagttctcatggtttcgggatgttgagttcgctaggcagactcttgctggtctgaacccttactctattaggcttgtgaccgagtggcctctgaggtctaacttggatcctaaggtttcaggccctcctgagagcgagattaccaaagagcttatcgagaacgagatcggcaacaacatgaccgttgagcaagcagtgcagcagaagaagctgttcatcctggattaccacgatctgctgctgccgtacgttaacaaggtgaacgagcttaagggcagcgtgttgtacggttctcggactatcttcttcctgactcctcacggaacccttaagcctcttgctattgagcttaccaggcctcctatcgatgataagccgcagtggaaagaggtgtacagccctaacaattggaacgctactggtgcttggctttggaagcttgctaaggctcatgtgctgagccacgattctggttaccatcagcttgtgtctcactggcttaggactcattgctgtaccgagccttacattatcgccaccaacaggcagctttctgctatgcatcctatctacaggctgctgcatcctcacttcaggtacaccatggaaatcaacgctctggctagggaagctctgatcaacgccaacggtattatcgagtcctcattcttcccgggcaagtacagcgttgagctgtcctctattgcttacggtgctgagtggcgtttcgatcaagaagctttgcctcagaacctgatcagcagaggtcttgctgaggaagatcctaacgagcctcacggtcttaagctggctattgaggattaccctttcgctaacgatggtctggtgctgtgggatattcttaagcagtgggtgaccaactacgtgaaccactactaccctcagaccaacctgatcgagagcgacaaagaattgcaggcttggtggtccgagatcaagaatgttggtcacggcgacaagaaagacgaaccttggtggcctgaacttaagacccctaacgatctgatcggcatcatcaccactatcgtctgggttacctctggtcatcatgctgctgtgaacttcggccagtactcttacggtggttacttccctaataggcctaccaccgctaggtctaagatgcctactgaggatcctactgctgaagagtgggagtggttccttaacaagcctgaagaggctctgctgcggtgtttcccatctcaaattcaggctactaaggtgatgaccatcctcgacgtgctgtctaaccattctcctgacgaagagtacatcggcgagaagattgagccttactgggctgaagatccggtgattaacgctgctttcgaggtgttctccggcaagcttaaagagcttgagggcatcatcgacgcccggaacaatgattctaagctgaacaataggaacggcgctggcgttatgccttacgagcttcttaagccttacagcgagcctggtgttactggaaagggtgtgccatacagcatcagcatctaggctttgagaccacgaagtg

SEQ ID NO: 28 - DNA, 67 bp, synthetic construct
  source 1..67: mol_type = other DNA
cactctgtggtctcaaatgtacccttacgacgttcctgattacgctggaggttgagaccacgaagtg

(19) United States
(12) Patent Application Publication
(10) Pub. No.: US 2025/0388916 A1
(43) Pub. Date: Dec. 25, 2025

(54) PLANTS FOR PRODUCING CANNABINOIDS

(71) Applicant: Commonwealth Scientific and Industrial Research Organisation, Acton (AU)

(72) Inventors: Rosangela DEVILLA, Acton (AU); Nathalie NIESNER, Acton (AU); Vivien ROLLAND, Acton (AU); Surinder SINGH, Acton (AU); Thomas Vanhercke, Acton (AU); Ingrid VENABLES, Acton (AU); Xue-Rong ZHOU, Acton (AU)

(73) Assignee: Commonwealth Scientific and Industrial Research Organisation, Acton (AU)

(21) Appl. No.: 18/722,386

(22) PCT Filed: Dec. 19, 2022
(86) PCT No.: PCT/AU2022/051536

§ 371 Date: Jun. 20, 2024

(30) Foreign Application Priority Data
Dec. 20, 2021 (AU) 2021904150

(51) Int. Cl. C12N 15/82; A61K 31/00; C07K 14/00; C12N 5/04; C12N 5/10; C12N 9/02; C12N 9/10; C12N 9/88; C12N 15/52; C12N 15/62; C12P 7/42

(52) U.S. Cl. CPC C12N 15/8214; A61K 31/658; C07K 14/00; C12N 5/04; C12N 5/10; C12N 9/0069; C12N 9/1029; C12N 9/88; C12N 15/52; C12N 15/62; C12P 7/42; C12Y 113/11; C12Y 203/01206; C12Y 404/01026; C07K 2319/00; C12N 2510/02

(57) ABSTRACT

The present invention relates to polynucleotides for the generation of genetically modified plants, algae or plastids thereof that are capable of producing cannabinoids. In an aspect, the present invention also relates to methods of producing cannabinoids.

### CROSS-REFERENCE TO RELATED APPLICATIONS

This application is a § 371 national stage of PCT International Application No. PCT/AU2022/051536, filed Dec. 19, 2022, claiming priority of Australian Patent Application No. AU2021904150, filed Dec. 20, 2021, the entire contents of each of which are hereby incorporated by reference into the subject application.

### REFERENCE TO SEQUENCE LISTING

This application incorporates-by-reference nucleotide and/or amino acid sequences which are present in the file named “240624_92429_96261_0087_SeqListing_AS.txt”, which is 239 kilobytes in size, and which was created on Jun. 20, 2024 in the IBM-PCT machine format, having an operating system compatibility with MS-Windows, which is contained in the text file filed Jun. 20, 2024 as part of this application.

### FIELD OF THE INVENTION

The present invention relates to polynucleotides for the generation of genetically modified plants, algae or plastids thereof that are capable of producing cannabinoids. In an aspect, the present invention also relates to methods of producing cannabinoids.

### BACKGROUND OF THE INVENTION

The plant Cannabis sativa has been utilised as a medicine over many centuries. For instance, Cannabis has previously been utilised in China to treat gout, malaria, digestive disorders, and menstrual pain and has been further utilised in Western medicine for the treatment of rheumatism and seizures (Bostwick, 2012; Russo, 2016; Kinghorn et al., 2017; Baron, 2018). Since that time, a growing number of cannabinoid medicinal drugs have been approved for human use, including nabilone in 1985, dronabinol in 1986, rimonabant in 2006 (in Europe; withdrawn in 2008), Sativex® in 2010, and Epidiolex® in 2018. A growing number of countries have also approved the use of Cannabis for treating a variety of medical conditions with cannabidiol (CBD) oil being a favoured mode of administration.

In Cannabis, the cannabinoid synthesis pathway begins with the precursor molecules olivetolic acid (OA) and geranyl-pyrophosphate (GPP), which combine to form cannabigerolic acid (CBGA) (Shoyama et al., 1975; Fellermeier and Zenk, 1998; Fellermeier et al., 2001; Gülck and Moller, 2020). CBGA serves as the precursor to most other cannabinoids and is converted to Δ9-tetrahydrocannabinolic acid (Δ9-THCA), cannabidiolic acid (CBDA), and cannabichromenic acid (CBCA) in Cannabis (FIG. 1). Because CBGA serves as the precursory molecule to other cannabinoids, it is normally found in very low quantities in Cannabis.

Enzymatically produced cannabinoids (including CBGA) are produced in their acidic form and are then decarboxylated to create the “active” form (i.e. THC (tetrahydrocannabinol), CBD (cannabidiol), CBC (cannabichromene), CBG (cannabigerol) etc). With the recent deregulation of CBD and other Cannabis-derived cannabinoids, there is growing interest in cannabinoid pharmacology (www.usda.gov/farmbill). In particular, in spite of having CBG as a common precursor, Δ9-THC, CBD, and CBC have dramatically different physiological effects. For instance, Δ9-THC is known to produce euphoria and appetite stimulation (Volkow et al., 2014), while CBD is non-euphorigenic and has been shown to have antiepileptic (Jones et al., 2010) and anti-inflammatory effects (Carrier et al., 2006).

Different approaches to the generation of cannabinoid compounds in plant hosts have been attempted. In WO 2021/081648, the authors used algae as a system for producing olivetolic acid (OA) which included the generation of an olivetolic acid synthase (OAS) and olivetolic acid cyclase (OAC) fusion protein, however the scaling up for commercial production is costly and requires high energy levels. The authors of WO 2018/200888 overexpressed a geranyl pyrophosphate:olivetolic acid geranyltransferase polypeptide to catalyse the production of cannabigerolic acid from geranyl pyrophosphate and olivetolic acid in yeast. However, yeast scaling is not predictable from small scale, and is associated with a lack of certainty of real recovery. Yeast also use geranyl pyrophosphate (GPP) for making isoprenoids so it is necessary to develop approaches to block consumption of the substrate necessary for cannabinoid production. Yet another approach involved the generation of components of the cannabinoid pathway in a heterologous plant, however the authors of this work were unable to detect the production of CBGA, despite the formation of OA and OA-glucoside (Gulck et al., 2020). The authors speculated that lack of CBGA may be due to glycosylation of OA, impeding the biosynthesis of downstream cannabinoid products, highlighting the difficulties for successful production of cannabinoids therefrom.

In response to increasing demand for medicinal uses of Cannabis, many countries have sought to legalise and decriminalise Cannabis by relaxing the laws relating to drug regulation, however the cultivation of Cannabis is still tightly controlled by strict regulations (Alharbi, 2020; Śledziński et al. 2021). For example, within the EU, territories control the varieties that may be grown based upon the THC content (Hazekamp, 2018). Although Cannabis varieties with reduced activity of the three major synthesis enzymes can accumulate higher levels of CBGA, the presence of euphorigenic THCA is still a confounding problem (Fellermeier and Zenk, 1998; Fellermeier et al., 2001). Breeding or modification of Cannabis to produce CBD only is not achievable, as THC levels are difficult to control. Furthermore, reliable efficient transformation and regeneration protocols for the modification of Cannabis are yet to be established (Schachtsiek et al., 2018; Deguchi et al., 2020). In addition, the production of the major cannabinoid is mainly limited to one organ, the female flower; as a result, the majority of the biomass is wasted.

These and other complexities related to cannabinoid metabolism has resulted in challenges in the development of robust and cost effective approaches for production of cannabinoids.

As a result, there is a need for the development of alternative strategies for the production of cannabinoids.

### SUMMARY OF THE INVENTION

The present inventors have demonstrated for the first time a functional cannabinoid biosynthesis pathway in a heterologous plant. In particular, the expression of components of the cannabinoid pathway in a heterologous plant successfully resulted in the synthesis of OA and CBGA. The inventors show that targeting of components of the cannabinoid pathway to the plastid of the host plant increases yields of OA, CBGA and their respective glycosylated forms. Significant increases in the levels of all four cannabinoids were also obtained when components of the cannabinoid biosynthetic pathway were overexpressed using an optimised vector for expression in the plastid.

The inventors therefore demonstrate for the first time the reconstitution of a functional cannabinoid biosynthetic pathway in a heterologous plant that results in OA and CBGA production. Such a system is applicable to the generation of a scalable production system, such as a plant biomass with industrial applicability.

The present inventors have thus developed polynucleotides that are useful in the generation of genetically modified plants, algae and plastids thereof for increasing cannabinoid production.

Thus, in a first aspect, there is provided a plastid of a genetically modified plant, part thereof or alga comprising at least one polypeptide selected from the group consisting of a polynucleotide encoding a polyketide synthase, a polyketide cyclase, an acyl-activating enzyme, a prenyltransferase, a plastid acyl-lipid thioesterase, a plastid lipase or a plastid tomato 13-lipoxygenase, wherein the at least one polypeptide increases the production of cannabigerolic acid (CBGA) in the presence of hexanoic acid (C6) and geranyl-pyrophosphate (GPP), when compared to a plastid of a wild-type plant, part thereof or alga.

In another aspect, the present invention provides a plastid of a genetically modified plant, part thereof or alga comprising at least one polypeptide selected from the group consisting of an acyl activating enzyme 1 (AAE1), an olivetol synthase (OLS) and an olivetolic acid cyclase (OAC),
wherein the at least one polypeptide increases the production of cannabigerolic acid (CBGA) in the presence of hexanoic acid (C6) and geranyl-pyrophosphate (GPP), when compared to a plastid of a wild-type plant, part thereof or alga.

In another aspect, the present invention provides a plastid of a genetically modified plant or part thereof comprising an acyl activating enzyme 1 (AAE1) polypeptide, an olivetol synthase (OLS) polypeptide and an olivetolic acid cyclase (OAC) polypeptide, optionally further comprising a plastid localised prenyltransferase polypeptide, preferably cannabigerolic acid synthase (CBGAS),
wherein the polypeptides increase the production of cannabigerolic acid (CBGA) in the presence of hexanoic acid (C6) and geranyl-pyrophosphate (GPP), when compared to a plastid of a wild-type plant or part thereof.

Optionally, the at least one polypeptide or polypeptides increase the production of olivetolic acid (OA) in the presence of hexanoic acid (C6), when compared to a plastid of a wild-type plant, part thereof or alga.

In an embodiment, the at least one polypeptide selected from the group consisting of AAE1, OLS and OAC, or polypeptides, are encoded by one or more polynucleotides, wherein the one or more polynucleotides, optionally each polynucleotide, encodes a fusion polypepide comprising a plastid transporting peptide, preferably a chloroplast transit peptide (CTP); and wherein the one or more polynucleotides are operably linked to a promoter capable of directing expression of the one or more polynucleotides in the plant or part thereof. In an embodiment, the at least one polypeptide selected from the group consisting of AAE1, OLS and OAC, or polypeptides, are encoded by one or more polynucleotides, wherein the polynucleotide is integrated into the plastidial genome and wherein the polynucleotide is operably linked to a promoter capable of directing expression of the polynucleotide in the plant or part thereof.

In yet another embodiment, the at least one polypeptide selected from the group consisting of AAE1, OLS and OAC, or polypeptides, are encoded by one or more polynucleotides, wherein the one or more polynucleotides are operably linked to a viral vector sequence capable of expressing the polynucleotide in a plant or part thereof, or is comprised in a viral vector capable of expressing the polynucleotide in a plant or part thereof; and optionally, wherein the polynucleotide encodes a fusion protein comprising a plastid transporting peptide.

Optionally, each polynucleotide encoding the polypeptide is operably linked to a viral vector sequence capable of expressing the polynucleotide in a plant or part thereof, or is comprised in a viral vector capable of increasing the polynucleotide expression; and the CBGA and/or OA production is enhanced.

In another embodiment, the plastid comprises the polypeptides AAE1, OLS and OAC, optionally further comprising a plastid localised prenyltransferase, preferably cannabigerolic acid synthase (CBGAS). In this embodiment, at least two polypeptides, optionally each polypeptide, comprises a plastid transporting peptide, preferably a chloroplast transit peptide (CTP).

In another aspect, the present invention provides a plastid of a genetically modified plant, part thereof or alga comprising at least one polynucleotide encoding an acyl activating enzyme 1 (AAE1), an olivetol synthase (OLS) and an olivetolic acid cyclase (OAC),
wherein the at least one polynucleotide is operably linked to a promoter which is capable of directing expression of the polynucleotide in the plastid;
wherein the at least one polypeptide encoded by the polynucleotide increases the production of cannabigerolic acid (CBGA) in the presence of hexanoic acid (C6) and geranyl-pyrophosphate (GPP), when compared to a plastid of a wild-type plant, part thereof or alga. Optionally, the at least one polypeptide encoded by the polynucleotide increases the production of olivetolic acid (OA) in the presence of hexanoic acid (C6), when compared to a plastid of a wild-type plant, part thereof or alga. In an embodiment, the plastid of a genetically modified plant, part thereof or alga comprises polynucleotides encoding AAE1, OLS and OAC and further comprises a polynucleotide encoding a plastid localised prenyltransferase, preferably cannabigerolic acid synthase (CBGAS).

In an embodiment, the polynucleotide is contained in a nucleic acid construct comprising sequences enabling integration of the polynucleotide into the genome of the plastid, and preferably not into the genome of the nucleus of the plant, part thereof or alga. Non-limiting examples of specific integration sites may include tmH/pbA, tmG/tmfM, ycf3/tmS, rbcL/accD, petA/psbJ, 5′rps12/clpP, petD/rpoA, ndhB/rps7, 3′rpsl2/tmV, tmV/rml6, rml6/tml, tml/tmA, tmN/tmR, and rp32/tmL.

In another aspect, there is provided a genetically modified plant, part thereof or alga comprising at least one polynucleotide selected from the group consisting of a a polynucleotide encoding a polyketide synthase, a polyketide cyclase, an acyl-activating enzyme, a prenyltransferase, a plastid acyl-lipid thioesterase, a plastid lipase or a plastid tomato 13-lipoxygenase,
wherein the at least one polynucleotide is operably linked to a promoter which is capable of directing expression of the polynucleotide in the plant, part thereof or alga, optionally wherein the at least one polynucleotide is codon optimised for expression in the plant, part thereof or alga, and
when expressed in the plant, part thereof or alga in the presence of hexanoic acid (C6) and geranyl-pyrophosphate (GPP), the polypeptide encoded by the at least one polynucleotide increases the production of cannabigerolic acid (CBGA) when compared to a wild-type plant, part thereof or alga.

In another aspect, the present invention provides a genetically modified plant, part thereof or alga comprising at least one polynucleotide selected from the group consisting of a polynucleotide encoding an acyl activating enzyme 1 (AAE1), a polynucleotide encoding an olivetol synthase (OLS) and a polynucleotide encoding an olivetolic acid cyclase (OAC), optionally further comprising a polynucleotide encoding a plastid localised prenyltransferase polypeptide, preferably cannabigerolic acid synthase (CBGAS),
wherein the at least one polynucleotide is operably linked to a promoter which is capable of directing expression of the polynucleotide in the plant, part thereof or alga, wherein the at least one polynucleotide is codon optimised for expression in the plant, part thereof or alga, and
when expressed in the plant, part thereof or alga in the presence of hexanoic acid (C6) and geranyl-pyrophosphate (GPP), the polypeptide encoded by the at least one polynucleotide increases the production of cannabigerolic acid (CBGA) when compared to a wild-type plant, part thereof or alga. Optionally, the at least one polypeptide increases the production of olivetolic acid (OA) in the presence of hexanoic acid (C6), when compared to a wild-type plant, part thereof or alga.

In another aspect, there is provided a genetically modified plant, part thereof or alga comprising at least one polynucleotide selected from the group consisting of a polynucleotide encoding an acyl activating enzyme 1 (AAE1), a polynucleotide encoding an olivetol synthase (OLS) and a polynucleotide encoding an olivetolic acid cyclase (OAC), optionally further comprising a polynucleotide encoding a plastid localised prenyltransferase polypeptide, preferably cannabigerolic acid synthase (CBGAS),
wherein the at least one polynucleotide is operably linked to a promoter which is capable of directing expression of the polynucleotide in the plant, part thereof or alga, and
when expressed in the plant in the presence of hexanoic acid (C6) and geranyl-pyrophosphate (GPP), the polypeptide encoded by the at least one polynucleotide increases the production of cannabigerolic acid (CBGA) when compared to a wild-type plant, part thereof or alga.

In another aspect, there is provided a genetically modified plant or part thereof comprising a polynucleotide encoding an acyl activating enzyme 1 (AAE1), a polynucleotide encoding an olivetol synthase (OLS) and a polynucleotide encoding an olivetolic acid cyclase (OAC), optionally further comprising a polynucleotide encoding a plastid localised prenyltransferase polypeptide, preferably cannabigerolic acid synthase (CBGAS),
wherein each polynucleotide is operably linked to a promoter which is capable of directing expression of the polynucleotide in the plant or part thereof,
wherein the polynucleotides are codon optimised for expression in the plant or part thereof, and
when expressed in the plant or part thereof in the presence of hexanoic acid (C6) and geranyl-pyrophosphate (GPP), the polypeptides encoded by the polynucleotides increase the production of cannabigerolic acid (CBGA) when compared to a wild-type plant or part thereof.

In an embodiment, the at least one polynucleotide, or polynucleotides are comprised within a vector, preferably a viral vector, optionally wherein the at least one polynucleotide, or polynucleotides encode/s a fusion polypepide comprising a plastid transporting peptide; and/or, the polypeptide encoded by the at least one polynucleotide or polynucleotides is/are expressed in the presence of a plastid localised prenyltransferase; wherein when expressed in the plant in the presence of hexanoic acid (C6) and geranyl-pyrophosphate (GPP), the polypeptide encoded by the at least one polynucleotide or polynucleotides increase/s the production of cannabigerolic acid (CBGA) compared to a wild-type plant, part thereof or alga. Optionally, the genetically modified plant, part thereof or alga further comprises a plastid localised prenyltransferase, preferably cannabigerolic acid synthase (CBGAS).

In an embodiment, the genetically modified plant is a vegetative plant part, preferably of a vascular plant.

In another embodiment, the genetically modified plant or part thereof is a high biomass plant, preferably selected from the group consisting of Acrocomia aculeata (macauba palm), Arabidopsis thaliana, Aracinis hypogaea (peanut), Astrocaryum murumuru (murumuru), Astrocaryum vulgare (tucuma), Attalea geraensis (Indaia-rateiro), Attalea humilis (American oil palm), Attalea oleifera (andaia), Attalea phalerata (uricuri), Attalea speciosa (babassu), Avena sativa (oats), Beta vulgaris (sugar beet), Brassica sp. Such as Brassica carinata, Brassica juncea, Brassica napobrassica, Brassica napus (canola), Camelina sativa (false flax), Cannabis sativa (hemp), Carthamus tinctorius (safflower), Caryocar brasiliense (pequi), Cocos nucifera (Coconut), Crambe abyssinica (Abyssinian kale), Cucumis melo (melon), Elaeis guineensis (African palm), Glycine max (soybean), Gossypium hirsutum (cotton), Helianthus sp. Such as Helianthus annuus (sunflower), Hordeum vulgare (barley), Jatropha curcas (physic nut), Joannesia princeps (arara nut-tree), Lemna sp. (duckweed) such as Lemna aequinoctialis, Lemna disperma, Lemna ecuadoriensis, Lemna gibba (swollen duckweed), Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Licania rigida (oiticica), Linum usitatissimum (flax), Lupinus angustifolius (lupin), Mauritia flexuosa (buriti palm), Maximiliana maripa (inaja palm), Miscanthus sp. Such as Miscanthus x giganteus and Miscanthus sinensis, Nicotiana sp. (tobacco) such as Nicotiana tabacum or Nicotiana benthamiana, Oenocarpus bacaba (bacaba-do-azeite), Oenocarpus bataua (pataua), Oenocarpus distichus (bacaba-de-leque), Oryza sp. (rice) such as Oryza sativa and Oryza glaberrima, Panicum virgatum (switchgrass), Paraqueiba paraensis (mari), Persea amencana (avocado), Pongamia pinnata (Indian beech), Populus trichocarpa, Ricinus communis (castor), Saccharum sp. (sugarcane), Sesamum indicum (sesame), Solanum tuberosum (potato), Sorghum sp. Such as Sorghum bicolor, Sorghum vulgare, Theobroma grandiforum (cupuassu), Trifolium sp., Trithrinax brasiliensis (Brazilian needle palm), Triticum sp. (wheat) such as Triticum aestivum and Zea mays (corn).

In another embodiment, the plant or part thereof is a Nicotiana sp., preferably Nicotiana benthamiana or Nicotiana tabacum.

In another embodiment, the plant or part thereof is a non-Cannabis plant. Alternatively, the plastid is not from an alga.

In an embodiment, at least one polypeptide or polynucleotides is/are active in the cytosol of the plant.

In another embodiment, where the plant, part thereof or alga comprises polynucleotides encoding AAE1, OLS and OAC, the polynucleotides are contained in the same or different nucleic acid constructs. Optionally, each polynucleotide is operably linked to a sequence capable of directing the polypeptide encoded by the polynucleotide to the plastid of the plant, algae or part thereof. Optionally, each polynucleotide encodes a fusion polypepide comprising a plastid transporting peptide. Optionally, one, more or all polynucleotides are integrated in the plastidial genome. In an embodiment, the polynucleotides encoding the OLS and OAC are fused.

In an embodiment, the polypeptide/s increase/s the production of CBGA in the vegetative parts of the plant or part thereof. In another embodiment, production of CBGA is increased by at least about 1.5-about 2 fold, about 2-about 2.5 fold, about 2.5-about 3.0 fold, about 3-about 3.5 fold, about 3.5-about 4.0 fold, about 4-about 4.5 fold, about 4.5-about 5.0 fold or about 5-about 5.5 fold or more when compared to a wild-type plant, part thereof, alga or plastid thereof.

In another embodiment, production of CBGA is increased at least about 10%-about 20%, about 20%-about 30%, about 30%-about 40%, about 40%-about 50%, about 50%-about 60%, about 60%-about 70%, about 70%-about 80%, about 80%-about 90%, about 90%-about 100%, about 100%-about 120%, about 120%-about 140%, about 140%-about 160%, about 160%-about 180%, about 180%-about 200%, about 200%-about 220%, about 220%-about 240%, about 240%-about 260%, about 260%-about 280%, about 280%-about 300%, about 300%-about 320%, about 320%-about 340%, about 340%-about 360%, about 360%-about 380%, about 400%-about 420% or more when compared to a wild-type plant, part thereof, alga, or plastid thereof.

In an embodiment, the increased production of CBGA in the plastid, alga, plant or part thereof is determinable by chromatography.

In an embodiment, the plastid is a chloroplast. In another embodiment, the plastid transporting peptide is a chloroplast targeting peptide, preferably a chloroplast transit peptide (CTP). In yet another embodiment, the CTP is a stroma targeting peptide preferably wherein the stroma targeting peptide is a Rubisco small subunit or a Rubisco large subunit, preferably the Rubisco small subunit. Preferably the Rubisco small subunit is located at the N terminus of the polypeptide.

In an embodiment, the plant, part thereof or alga further comprises a polynucleotide encoding a prenyltransferase, preferably cannabigerolic acid synthase (CBGAS), wherein the polynucleotide encodes a fusion polypepide comprising a plastid transporting peptide. In another embodiment, the plant, part thereof or alga further comprises at least one polynucleotide selected from the group consisting of a polynucleotide encoding a acyl-lipid thioesterase (ALT4), a polynucleotide encoding a plastid lipase 1 (PLIP1) and a polynucleotide encoding a tomato 13-lipoxygenase (TomLoxC), optionally wherein the polynucleotide encodes a fusion polypepide comprising a plastid transporting peptide.

In another embodiment, the plastid further comprises a polypeptide encoded by a prenyltransferase, preferably a cannabigerolic acid synthase (CBGAS). In another embodiment the cannabigerolic acid synthase (CBGAS) is integrated into the plastid genome. In yet another embodiment the cannabigerolic acid synthase (CBGAS) is expressed in a viral vector. In another embodiment, the plastid further comprises at least one polypeptide selected from the group consisting of an acyl-lipid thioesterase (ALT4), a plastid lipase 1 (PLIP1) and a tomato 13-lipoxygenase (TomLoxC).

In another aspect, the present invention provides a nucleic acid construct encoding a polypeptide for expression in a plant, part thereof or alga, the nucleic acid construct comprising one or more or all of a polynucleotide encoding a polyketide synthase, a polyketide cyclase, an acyl-activating enzyme, a prenyltransferase, a plastid acyl-lipid thioesterase, a plastid lipase and a plastid tomato 13-lipoxygenase;
wherein the polynucleotide is operably linked to a promoter which is capable of directing expression of the one or more or all polynucleotide/s in the plant, part thereof or alga,
optionally, wherein the one or more or all polynucleotides are operably linked to a polynucleotide encoding a plastid transporting peptide;
optionally wherein the nucleic acid construct encodes a fusion polypepide comprising the plastid transporting peptide, and
when expressed, the polypeptide encoded by the polynucleotide increases the production of cannabigerolic acid (CBGA) compared to a wild-type plant, part thereof or alga. Optionally, the polynucleotide increases the production of olivetolic acid (OA), when compared to a wild-type plant, part thereof or alga.

In an embodiment, the nucleic acid construct is for expression in a plastid of the plant, part thereof or alga. In another embodiment, the nucleic acid construct comprises a polynucleotide encoding a silencing suppressor polypeptide, preferably a p19 silencing suppressor polypeptide.

In yet another embodiment, the nucleic acid construct is comprised within a vector suitable for expression in a plant, part thereof or alga. In another embodiment, the nucleic acid construct is operably linked to components of a viral vector suitable for expression in a plant, part thereof or alga. In an embodiment, the viral vector is a geminivirus, more preferably the viral vector is BeYDV. In an alternative embodiment the nucleic acid construct is operably linked to viral vector replication sequences. In a further embodiment the viral vector replication sequence includes the long intergenic region (LIR), short intergenic sequence (SIR) and Rep/RepA, preferably wherein the viral vector replication sequences are from a geminivirus.

In an embodiment:
i) the polynucleotide encoding a polyketide synthase is olivetol synthase (OLS);
ii) the polynucleotide encoding an acyl-activating enzyme is acyl activating enzyme 1 (AAE1);
iii) the polynucleotide encoding an polyketide cyclase is olivetolic acid cyclase (OAC);
   optionally wherein the nucleic acid construct comprises a polynucleotide encoding a silencing suppressor polypeptide, preferably a p19 silencing suppressor polypeptide, wherein one or more or all polynucleotides are operably linked to a promoter which is capable of directing expression of the polypeptide encoded by the polynucleotide in a plant, part thereof or alga;
   optionally, wherein the one or more or all polynucleotides are operably linked to a polynucleotide encoding a plastid transporting peptide; and
   optionally wherein the nucleic acid construct encodes a fusion polypepide comprising a plastid transporting peptide.

In an embodiment, the nucleic acid construct is suitable for transient expression in a plant, plant part, alga or cell thereof, for instance by use of an inducible promoter system described herein or known in the art. In another embodiment, the nucleic acid construct is suitable for stable expression in a plant, plant part, alga or cell thereof.

In an embodiment, production of CBGA is increased by at least about 1.5-about 2 fold, about 2-about 2.5 fold, about 2.5-about 3.0 fold, about 3-about 3.5 fold, about 3.5-about 4.0 fold, about 4-about 4.5 fold, about 4.5-about 5.0 fold or about 5-about 5.5 fold or more when the polynucleotide/s are expressed in a plant or part thereof, compared to a wild-type plant, part thereof or alga.

In another embodiment, production of CBGA is increased at least about 10%-about 20%, about 20%-about 30%, about 30%-about 40%, about 40%-about 50%, about 50%-about 60%, about 60%-about 70%, about 70%-about 80%, about 80%-about 90%, about 90%-about 100%, about 100%-about 120%, about 120%-about 140%, about 140%-about 160%, about 160%-about 180%, about 180%-about 200%, about 200%-about 220%, about 220%-about 240%, about 240%-about 260%, about 260%-about 280%, about 280%-about 300%, about 300%-about 320%, about 320%-about 340%, about 340%-about 360%, about 360%-about 380%, about 400%-about 420% or more when the polynucleotide/s are expressed in a plant or part thereof, compared to a wild-type plant, part thereof or alga.

In another embodiment, the polypeptide/s are preferably for expression in vegetative parts and/or seeds of a vascular plant, preferably leaves, more preferably in the chloroplasts of the leaves.

In another aspect, the present invention provides a fusion polypeptide for expression in a plant, part thereof or alga, comprising:
i) a polypeptide selected from the group consisting of a polyketide synthase, a polyketide cyclase, an acyl-activating enzyme, a a prenyltransferase, a plastid acyl-lipid thioesterase, a plastid lipase or a plastid tomato 13-lipoxygenase, and
ii) a plastid transporting sequence,
   wherein the polypeptide of (i) increases the production of cannabigerolic acid (CBGA) in the presence of hexanoic acid (C6) and geranyl-pyrophosphate (GPP), when compared to a wild-type plant or alga. Optionally, the fusion polypeptide increases the production of olivetolic acid (OA), when compared to a wild-type plant, part thereof or alga.

In an embodiment, i) the polypeptide selected from the group consisting of a polyketide synthase, polyketide cyclase, an acyl-activating enzyme, a prenyltransferase, a plastid acyl-lipid thioesterase, a plastid lipase or a plastid tomato 13-lipoxygenase is encoded by a polynucleotide, and ii) a plastid transporting sequence is encoded by a polynucleotide, preferably encoding a chloroplast transit peptide. In another embodiment, the fusion polypeptide is for expression in a plastid of the plant, part thereof or alga, preferably for expression in a plastid of vegetative parts and/or seeds of a vascular plant, preferably plastids of leaves, more preferably in the chloroplasts of the leaves.

In an embodiment, the polyketide synthase is olivetol synthase (OLS), the polyketide cyclase is olivetolic acid cyclase (OAC), the acyl-activating enzyme is acyl activating enzyme 1 (AAE1), the plastid acyl-lipid thioesterase is ALT4, the plastid lipase is PLIP1, the plastid tomato 13-lipoxygenase is TomLoxC and the prenyltransferase is cannabigerolic acid synthase (CBGAS), preferably wherein the plastid transporting sequence is a chloroplast transit peptide (CTP).

In another embodiment, one or more or all of:
i) the polynucleotide encoding AAE1 comprises a nucleotide sequence which is at least 70%, at least 75%, at least 80%, at least 85%, at least 90%, at least 95%, at least 96%, at least 97%, at least 98%, at least 99%, at least 99.5% at least 99.8% or identical to a sequence set forth as any one of SEQ ID NO:5, SEQ ID NO:6 and SEQ ID NO:7;
ii) the polynucleotide encoding OLS comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to a sequence set forth as any one of SEQ ID NO:8, SEQ ID NO:9 and SEQ ID NO:10;
iii) the polynucleotide encoding OAC comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to a sequence set forth as any one of SEQ ID NO:11, SEQ ID NO:12 and SEQ ID NO:13;
iv) the polynucleotide encoding the plastid transporting peptide comprises a sequence which is at least 70%, at least 75%, at least 80%, at least 85%, at least 90%, at least 95%, at least 96%, at least 97%, at least 98%, at least 99%, at least 99.5%, at least 99.8% or identical to the sequence set forth as SEQ ID NO:15.

In another embodiment, the polynucleotide encoding a prenyltransferase comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to a sequence set forth as SEQ ID NO:14.

In another embodiment,
i) the polynucleotide encoding ALT4 comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to a sequence set forth as any one of SEQ ID NO:24 or SEQ ID NO:25;
ii) the polynucleotide encoding PLIP1 comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to the sequence set forth as SEQ ID NO:26; and
iii) the polynucleotide encoding TomLoxC comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to the sequence set forth as SEQ ID NO:27.

In another aspect, the present invention provides a plant, part thereof, alga, plastid, or cell thereof comprising a polypeptide or fusion polypeptide of the invention.

In another aspect, the present invention provides a method of producing cannabigerolic acid (CBGA) in a plant, part thereof or alga, the method comprising cultivating a plant, part thereof or alga comprising a nucleic acid construct of the invention under conditions sufficient for expression of the polypeptide in the plant, part thereof or alga, wherein when expressed in the presence of hexanoic acid (C6) and geranyl-pyrophosphate (GPP), the polypeptides increase the production of cannabigerolic acid (CBGA) in the plant, part thereof or alga when compared to a wild type plant, part thereof or alga. Optionally, the method further comprises introducing a nucleic acid construct of the invention into the plant, part thereof or alga. Optionally, the method produces olivetolic acid (OA), when compared to a wild-type plant, part therof or alga.

In another aspect, the present invention provides a method of producing olivetolic acid (OA) in a plant, part thereof or alga, the method comprising cultivating a plant, part thereof or alga comprising a nucleic acid construct of the invention under conditions sufficient for expression of the polypeptide in the plant, part thereof or alga, wherein when expressed in the presence of olivetol synthase (OAS) and/or olivetolic acid cyclase (OAC), OA is produced in the plant, part thereof or or alga when compared to a wild type plant, part thereof or alga.

In another aspect, the present invention provides a method of producing cannabigerolic acid (CBGA) in a plant, part thereof or alga, the method comprising cultivating a plant, part thereof or alga comprising a nucleic acid construct of the invention under conditions sufficient for expression of the polypeptide in the plant, part thereof or alga, wherein when expressed in the presence of cannabigerolic acid synthase (CBGAS), CBGA is produced in the plant, part thereof or alga when compared to a wild type plant, part thereof or alga.

In another aspect, the present invention provides a method of producing C6-CoA from C6 in a plant, part thereof or alga, the method comprising cultivating a plant, part thereof or alga comprising a nucleic acid construct of the invention under conditions sufficient for expression of the polypeptide/s in the plant, part thereof or alga, wherein when expressed in the presence of acyl activating enzyme 1 (AAE1), C6-CoA is produced in the plant, part thereof or alga when compared to a wild type plant, part thereof or alga.

In an embodiment, the method further comprises providing exogenous C6, malonyl-CoA and/or GPP to the plant, part thereof or alga, optionally in the form of plant feed, amino acid supplement or C6 and/or GPP substrate.

In an embodiment, the polypeptide/s described herein is/are expressed in the plastid of the plant, part thereof or alga.

In an embodiment, the CBGA is produced in the vegetative parts of the plant and/or seeds, preferably wherein the vegetative part is an aerial part comprising leaves of the plant. In an embodiment, increased production of CBGA in the vegetative parts of the plant is determinable by chromatography.

In an embodiment, the method further comprises the production of a cannabinoid, the method comprising:
i) recovering the CBGA produced by a method of the invention; and
ii) optionally feeding the CBGA to a modified microorganism, preferably yeast or bacteria; and
iii) chemically, physically or biochemically converting the CBGA, optionally by using the CBGA as a feedstock, thereby producing the cannabinoid.

In an embodiment, the method comprises the production of at least one of cannbigerol (CBG), tetrahydrocannabinolic acid (THCA), cannabidiolic acid (CBDA) and cannabichromenic acid (CBCA), preferably cannabidiolic acid (CBDA) optionally comprising production of tetrahydrocannabinol (THC), cannabidiol (CBD), cannabinol (CBN) and cannabichromene (CBC), preferably cannbigerol (CBG) or cannabidiol (CBD).

In another embodiment, the method comprises a step of CBGA decarboxylation after recovery of the CBGA containing extract, preferably by incubation of CBGA with heat or light.

In another aspect, the present invention provides cannabigerol (CBG) produced from a method of the invention.

In another aspect, the present invention provides cannabigerolic acid (CBGA) produced from a method of the invention. In an embodiment, the CBGA is substantially glycosylated. In another embodiment, the CBGA is substantially free of glycosylation.

In another aspect, the present invention provides cannabidiolic acid (CBDA) or cannabidiol (CBD) produced from a method of the invention.

In another aspect, the present invention provides cannabichromenic acid (CBCA) or cannabichromene (CBC) produced from a method of the invention.

In another aspect, the present invention provides olivetolic acid (OA) produced from a method of the invention.

In another aspect, the present invention provides tetrahydrocannabinolic acid (THCA) or tetrahydrocannabinol (THC) produced from a method of the invention.

In another aspect, the present invention provides a method of producing a genetically modified plant, the method comprising:
i) regenerating a plant from a plant cell transformed with a nucleic acid construct of the invention;
ii) optionally harvesting seed from the plant; and/or
iii) optionally producing one or more progeny plants from the genetically modified plants, thereby producing the genetically modified plant.

In another aspect, the present invention provides a method of producing a genetically modified seed, the method comprising:
i) regenerating a plant from a plant cell transformed with a nucleic acid construct of the invention; and
ii) harvesting seed from the plant, thereby producing a genetically modified seed.

In an embodiment, the method further comprises introducing a nucleic acid construct of the invention into the plant or cell thereof.

In an embodiment, the encoded polypeptide is expressed in the vegetative parts of the plant, preferably the aerial vegetative parts, more preferably the leaves. In another embodiment, the plant is a vascular plant, preferably of the genus Nicotiana, more preferably Nicotiana benthamiana or Nicotiana tabacum.

In another aspect, the present invention provides a method of producing a genetically modified plant or alga, the method comprising:
i) crossing two parental plants or alga, wherein at least one plant or alga comprises a genetic modification(s) introduced by a nucleic acid construct of the invention,
ii) screening one or more progeny plants or alga from the cross in i) for the presence or absence of the genetic modification(s), and
iii) selecting a progeny plant or alga which comprise the genetic modification(s), thereby producing the plant or alga.

In an embodiment, the method further comprises:
iv) crossing a first genetically modified parent plant with a second parent plant, wherein the first genetically modified parent plant is a plant that comprises a genetic modification(s) introduced by a nucleic acid construct described herein; and
v) backcrossing a progeny plant of the cross of step (iv) with a plant of the same genotype as the second parent plant to produce a plant with a majority of the genotype of the second parent and comprising said genetic modification.

In an embodiment, the vegetative parts, or aerial vegetative parts are capable of photosynthesis, preferably wherein the leaves of the plant comprise the genetic modification.

In another aspect, the present invention provides a genetically modified and/or recombinant plant or alga cell for producing cannabigerolic acid (CBGA) comprising a nucleic acid construct of the invention. Optionally, the genetically modified and/or recombinant plant or alga cell produces olivetolic acid (OA).

In another aspect, the present invention provides a genetically modified plant, part thereof or alga for producing cannabigerolic acid (CBGA) comprising a nucleic acid construct of the invention. Optionally, the genetically modified plant, part thereof or alga produces olivetolic acid (OA).

In another aspect, the present invention provides a plant part of the genetically modified plant of the invention. Optionally, the plant part is a seed comprising the genetic modification(s).

In another aspect, the present invention provides a method of producing a plant part from a genetically modified plant, the method comprising,
i) growing a plant according to a method of the invention, and
ii) harvesting the plant part.

In another aspect, the present invention provides use of a nucleic acid construct of the invention to produce a recombinant plant or alga cell and/or a genetically modified plant and/or alga.

In another aspect, the present invention provides a crop or population of plants or algae comprising:
(i) plants or algae, plastids, nucleic acid constructs or fusion polypeptides of the invention; or
(ii) plants or algae, plastids, nucleic acid constructs or fusion polypeptides of the invention and wild-type plants or algae. In an embodiment, the crop or population of plants comprises vegetative plant parts, preferably of vascular plants, more preferably of Nicotiana, most preferably of Nicotiana benthamiana or Nicotiana tabacum plants. In a further embodiment, the crop or population of plants further comprises non-vascular plants.

In an embodiment, the plants or algae comprise a higher yield of CBGA, optionally OA, when compared to wild type plants or algae, wherein the CBGA and/or OA is optionally determinable by chromatography.

In another aspect, the present invention provides an industrial plant biomass obtained from leaves of genetically modified plants of the invention, wherein the leaves of the plant biomass comprise a higher yield of CBGA, optionally OA, when compared to leaves of wild type plants, optionally determinable by chromatography.

In another aspect, the present invention provides a composition comprising a plastid, nucleic acid construct or plant cell of the invention and one or more acceptable carriers.

In another aspect, there is provided a process for identifying a polynucleotide encoding a polypeptide for producing cannabigerolic acid (CBGA), optionally OA, in a plant, part thereof, alga or plastid or cell thereof, the process comprising:
i) obtaining a nucleic acid construct of the invention,
ii) introducing the nucleic acid construct into plant, part thereof, alga or plastid or cell thereof,
iii) determining whether the level of CBGA, optionally OA, is increased relative to a corresponding wild-type plant, part thereof, alga or plastid or cell thereof lacking the nucleic acid construct, and
iv) optionally, selecting a polynucleotide, which when expressed produces a polypeptide suitable for producing CBGA, optionally OA. Preferably, the CBGA is produced in plastids of the vegetative parts of the plant, optionally determinable by chromatography.

In another aspect, there is provided a process for producing an industrial product, the process comprising the steps of:
(i) obtaining a plant, part thereof, alga or a plastid of the invention;
(ii) processing the plant, part thereof, alga or plastid;
(iii) converting at least some of the cannabinoid in the plant, part thereof, alga, or plastid of step (i), or in the processed plant, part thereof, alga or plastid, obtained by step (ii) by applying heat, chemical, or enzymatic means, or any combination thereof, to the cannabinoid in situ; and
(iv) recovering the cannabinoid, thereby producing the industrial product.

In a preferred embodiment, the industrial product is cannabigerolic acid (CBGA) or olivetolic acid (OA). In a preferred embodiment, the industrial product is cannabigerolic acid (CBGA) or cannabigerol (CBG).

In another aspect, there is provided a process for producing extracted cannabigerolic acid (CBGA), the process comprising the steps of:
(i) obtaining a plant, part thereof, alga or plastid of the invention,
(ii) extracting CBGA from the plant, part thereof, alga or plastid; and
(iii) recovering the extracted CBGA, thereby producing extracted CBGA.

Optionally, the extracted CBGA comprises olivetolic acid (OA).

In an embodiment, the extract process of step (ii) comprises one or more or all of (a) milling of dry material, (b) solvent (e.g., Ethanol, hydrocarbon) or supercritical CO2 extraction, (c) winterization & filtering, (d) drying, (e) distillation and/or (f) chromatography.

In another aspect, there is provided a process of producing a cannabigerolic acid (CBGA) enriched extract, the process comprising the steps of:
(i) obtaining a plant, part thereof, alga of or plastid of the invention;
(ii) processing the plant, part thereof, alga or plastid of step (i);
(iii) centrifuging the processed plant, part thereof, alga or plastid from step (ii) to obtain extracts;
(iv) filtering the extract obtained by step (iii);
(v) drying the filtered extract of step (iv) and resuspending in buffer solution;
(vi) purifying the extract of step (v) using a solid-phase extraction column; and
(vii) recovering the CBGA enriched extract,
   thereby producing the CBGA enriched extract. Optionally, the extracted CBGA comprises olivetolic acid (OA).

In another aspect, there is provided a method for producing a pharmaceutical composition comprising:
(i) obtaining an extract or a cannabinoid of the invention; and
(ii) formulating the extract or cannabinoid with one or more pharmaceutically acceptable carriers,
   thereby producing a pharmaceutical composition.

In another aspect, there is provided a pharmaceutical composition obtained from a method of the invention.

In another aspect, there is provided a method of treating and/or preventing a condition or disease responsive to cannabinoid treatment in a subject comprising administering a therapeutically effective amount of a cannabinoid of the invention, an extract or pharmaceutical composition of the invention to the subject, thereby treating and/or preventing a condition or disease responsive to cannabinoid treatment.

In another aspect, there is provided use of a therapeutically effective amount of a cannabinoid of the invention, an extract or pharmaceutical composition of the invention in the manufacture of a medicament for treating a condition or disease responsive to cannabinoid treatment.

In another aspect, there is provided a therapeutically effective amount of a cannabinoid of the invention, extract of the invention or a pharmaceutical composition of the invention for use in treating a condition or disease responsive to cannabinoid treatment.

In an embodiment, the condition or disease is selected from the group consisting of chronic pain, neuropathic pain, cancer, nausea and/or vomiting associated with cancer chemotherapy, lack of appetite, multiple sclerosis, spasticity associated with multiple sclerosis or spinal cord injury epilepsy, Parkinson's disease, anorexia and/or weight loss, irritable bowel syndrome, Tourette syndrome, amyotrophic lateral sclerosis, Huntington's disease, dystonia, dementia, glaucoma, dermatitis, acne, microbial infection, traumatic brain injury and/or intracranial haemorrhage, addiction, anxiety, depression, sleep disorders, post-traumatic stress disorder, microbial infection including methicillin-resistant Staphylococcus aureus (MRSA) and biofilm producing organisms, dermatitis, acne, schizophrenia and other psychoses.

In another aspect, there is provided use of a plant, part thereof, alga or plastid of the invention in the manufacture of an industrial product, preferably a cannabinoid, more preferably cannabigerolic acid (CBGA) and/or cannbigerol (CBG).

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Cannabinoid biosynthesis pathway in C. sativa trichomes.

FIG. 2. OA glycosylation in infiltrated N. benthamiana leaves. A LC-MS chromatogram of OA standard. B LC-MS chromatogram of a N. benthamiana leaf extract following infiltration with OA.

FIG. 3. CBGA glycosylation in infiltrated N. benthamiana leaves. A LC-MS chromatogram of CBGA standard. B LC-MS chromatogram of N. benthamiana leaves following infiltration with CBGA.

FIG. 4. Functional CBGA biosynthesis pathway reconstituted in N. benthamiana spanning both cytosol and chloroplast.

FIG. 5. Functional CBGA biosynthesis pathway reconstituted in N. benthamiana in the chloroplast.

FIG. 6. Biosynthesis of OA in N. benthamiana leaves. CsAAE1, CsOLS, CsOAC, and CsCBGAS were co-infiltrated together with C6 and GPP as substrates for the recombinant cannabinoid biosynthesis pathway.

FIG. 7. Biosynthesis of glycosylated OA derivative in N. benthamiana leaves. CsAAE1, CsOLS, CsOAC, and CsCBGAS were co-infiltrated together with C6 and GPP as substrates for the recombinant cannabinoid biosynthesis pathway.

FIG. 8. Biosynthesis of CBGA in N. benthamiana leaves. CsAAE1, CsOLS, CsOAC, and CsCBGAS were co-infiltrated together with C6 and GPP as substrates for the recombinant cannabinoid biosynthesis pathway.

FIG. 9. Biosynthesis of glycosylated CBGA derivative in N. benthamiana leaves. CsAAE1, CsOLS, CsOAC, and CsCBGAS were co-infiltrated together with C6 and GPP as substrates for the recombinant cannabinoid biosynthesis pathway.

FIG. 10. Subcellular localization of CsAAE1, CsOLS, and CsOAC in N. benthamiana. Genes were fused at the 3′ end with the coding sequence of the mNeonGreen fluorescent protein and contained either the Rubisco SSU chloroplast targeting sequence (‘tp’) (left two columns) or the hemagglutinin epitope (‘HA’) (right two columns) as fusions at the 5′ end. For each construct (line), the left panel shown mNeonGreen fluorescence and the right panel shown mNeonGreen fluorescence together with chloroplast autofluorescence.

FIG. 11. Levels of OA (A) and CBGA (B) in N. benthamiana leaves. CsAAE1, CsOLS, CsOAC, and CsCBGAS were co-expressed either in the cytosol using a standard binary expression vector (Cyto), targeted to the chloroplast using a standard binary expression vector (Plast), localized in the cytosol using a deconstructed viral expression vector (Cyto VV) or targeted to the chloroplast using a deconstructed viral vector (Plast VV). OA and CBGA biosynthesis genes were co-infiltrated together with C6 and GPP as substrates for the recombinant cannabinoid biosynthesis pathway. Both free form and glycosylated derivatives were detected for OA and CBGA. P19+GFP, negative control. Relative amounts of products are shown as peak area.

FIG. 12. LC-MS chromatograms of N. benthamiana leaves, infiltrated with BeYDV-adapted expression vectors containing the coding sequences for OA and CBGA biosynthesis genes. A, C Cytosolic localization of CsAAE1, CsOLS and CsOAC. B, D Chloroplast targeting of CsAAE1, CsOLS and CsOAC.

FIG. 13. Biosynthesis of OA in N. benthamiana leaves. Chlorplast targeting CsAAE1, CsOLS, and CsOAC were co-expressed either with C6 substrate (Plast VV) or with AtALT4 (Plast VV+ALT) but no exogenous C6. Both free OA form and glycosylated derivative OA form were detected. P19+GFP, negative control. Relative amounts of products are shown as peak area and are based on four independent biological repeats.

### KEY TO THE SEQUENCE LISTING

SEQ ID NO: 1—Amino acid sequence of C. sativa enzyme CsAAE1.

SEQ ID NO: 2—Amino acid sequence of C. sativa enzyme CsOLS.

SEQ ID NO: 3—Amino acid sequence of C. sativa enzyme CsOAC.

SEQ ID NO: 4—Amino acid sequence of C. sativa enzyme CsCBGAS.

SEQ ID NO: 5—Codon optimised nucleotide sequence of C. sativa enzyme CsAAE1, designed as a GG level 0 module without N- or C-terminal fusions.

SEQ ID NO: 6—Codon optimised nucleotide sequence of C. sativa enzyme CsAAE1, designed as a GG level 0 module for N-terminal fusion.

SEQ ID NO: 7—Codon optimised nucleotide sequence of C. sativa enzyme CsAAE1, designed as a GG level 0 module for N and C-terminal fusions.

SEQ ID NO: 8—Codon optimised nucleotide sequence of C. sativa enzyme CsOLS, designed as a GG level 0 module without N- or C-terminal fusions.

SEQ ID NO: 9—Codon optimised nucleotide sequence of C. sativa enzyme CsOLS, designed as a GG level 0 module for N-terminal fusion.

SEQ ID NO: 10—Codon optimised nucleotide sequence of C. sativa enzyme CsOLS, designed as a GG level 0 module for N and C-terminal fusions.

SEQ ID NO: 11—Codon optimised nucleotide sequence of C. sativa enzyme CsOAC, designed as a GG level 0 module without N- or C-terminal fusions.

SEQ ID NO: 12—Codon optimised nucleotide sequence of C. sativa enzyme CsOAC, designed as a GG level 0 module for N-terminal fusion.

SEQ ID NO: 13—Codon optimised nucleotide sequence of C. sativa enzyme CsOAC, designed as a GG level 0 module for N- and C-terminal fusions.

SEQ ID NO: 14—Codon optimised nucleotide sequence of C. sativa enzyme CsCBGAS, designed as a GG level 0 module without N- or C-terminal fusions.

SEQ ID NO: 15—Nucleotide sequence encoding Rubisco small subunit targeting peptide; designed as a GG level 0 module as N-terminal fusion.

SEQ ID NO: 16—Nucleotide sequence encoding mNeonGreen; designed as a GG level 0 module as C-terminal fusion.

SEQ ID NO: 17—Nucleotide sequence encoding EN38510; designed as a GG level 0 promoter module, long intergenic region (LIR) sequence underlined.

SEQ ID NO: 18—Nucleotide sequence encoding EN38509; designed as a GG level 0 5′UTR module.

SEQ ID NO: 19—Nucleotide sequence encoding EN38511; designed as GG level 0 terminator module, LIR sequence underlined, SIR sequence in bold, Rep/RepA sequence in Italic.

SEQ ID NO: 20—Amino acid sequence of Arabidopsis thaliana enzyme AtALT4.1.

SEQ ID NO: 21—Amino acid sequence of Arabidopsis thaliana enzyme AtALT4.2.

SEQ ID NO: 22—Amino acid sequence of Arabidopsis thaliana enzyme AtPLIP1.

SEQ ID NO: 23—Amino acid sequence of TomloxC.

SEQ ID NO: 24—Codon optimised nucleotide sequence of Arabidopsis thaliana enzyme AtALT4.1, designed as a GG level 0 module without N- or C-terminal fusions.

SEQ ID NO: 25—Codon optimised nucleotide sequence of Arabidopsis thaliana enzyme AtALT4.2, designed as a GG level 0 module without N- or C-terminal fusions.

SEQ ID NO: 26—Codon optimised nucleotide sequence of Arabidopsis thaliana enzyme AtPLIP1, designed as a GG level 0 module without N- or C-terminal fusions.

SEQ ID NO: 27—Codon optimised nucleotide sequence of TomloxC, designed as a GG level 0 module without N- or C-terminal fusions.

SEQ ID NO: 28—Nucleotide sequence encoding EN38113 hemagglutinin epitope; designed as a GG level 0 module as N-terminal fusion.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, plant molecular biology, plant cannabinoid synthesis, protein chemistry, and biochemistry).

Unless otherwise indicated, the recombinant polynucleotide, polypeptide, cell and plant culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present) and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10%, more preferably 5%, more preferably 1%, of the particular term.

“Wild type”, as used herein, refers to a cell, plastid, tissue or plant that has not been modified according to the invention. For example, in an embodiment, there is provided a genetically modified plant or part thereof comprising a polynucleotide encoding an acyl activating enzyme 1 (AAE1), a polynucleotide encoding an olivetol synthase (OLS) and a polynucleotide encoding an olivetolic acid cyclase (OAC) that when expressed, can increase the production of cannabigerolic acid (CBGA) compared to a wild-type plant or part thereof. In this embodiment, the wild type plant does not comprise a polynucleotide encoding an acyl activating enzyme 1 (AAE1), a polynucleotide encoding an olivetol synthase (OLS) and a polynucleotide encoding an olivetolic acid cyclase (OAC) but will otherwise be similar in genotype and phenotype when compared to the genetically modified plant or part thereof. Wild-type cells, plastids tissue or plants may be used as controls to compare levels of expression of an exogenous nucleic acid or the extent and nature of trait modification with cells, plastids, tissue or plants modified as described herein.

### Cannabinoid Biosynthesis Pathway

In Cannabis, cannabinoids are produced from the common metabolite precursors geranylpyrophosphate (GPP) and hexanoyl-CoA by the action of three polypeptides. In particular, hexanoyl-CoA and malonyl-CoA combine to form a 12-carbon tetraketide intermediate by a tetraketide synthase (TKS) (olivetolic acid synthase; OAS) polypeptide. This tetraketide intermediate is then cyclized by an olivetolic acid cyclase (OAC) polypeptide to produce olivetolic acid (OA). OA is then prenylated with the common isoprenoid precursor GPP by a geranyltransferase polypeptide (e.g., a CsPT4 polypeptide) to produce cannabigerolic acid (CBGA). Various synthase polypeptides then convert CBGA into other cannabinoids. For instance, tetrahydrocannabinolic acid (THCA) synthase produces THCA and cannabidiolic acid (CBDA) synthase produces CBDA. In the presence of heat or light, the acidic cannabinoids undergo decarboxylation whereby cannabigerol (CBG) is produced from CBGA, tetrahydrocannabidiol (THC) is produced from THCA and cannabidiol (CBD) is produced from CBDA.

Geranyl-pyrophosphate (GPP) and hexanoyl-CoA can be generated through several pathways including via amino acid supplementation or via the mevalonate (MVA) pathway. The term “mevalonate pathway” or “MVA pathway,” refers to the biosynthetic pathway that converts acetyl-CoA to isopentenyl pyrophosphate (IPP) and dimethylallyl pyrophosphate (DMAPP). The mevalonate pathway comprises polypeptides that catalyze the following steps: (a) condensing two molecules of acetyl-CoA to generate acetoacetyl-CoA (e.g., by action of an acetoacetyl-CoA thiolase polypeptide); (b) condensing acetoacetyl-CoA with acetyl-CoA to form hydroxymethylglutaryl-CoA (HMG-CoA) (e.g., by action of a HMG-CoA synthase (HMGS) polypeptide); (c) converting HMG-CoA to mevalonate (e.g., by action of a HMGCoA reductase (HMGR) polypeptide); (d) phosphorylating mevalonate to mevalonate 5-phosphate (e.g., by action of a mevalonate kinase (MK) polypeptide); (e) converting mevalonate 5-phosphate to mevalonate 5-pyrophosphate (e.g., by action of a phosphomevalonate kinase (PMK) polypeptide); (f) converting mevalonate 5-pyrophosphate to isopentenyl pyrophosphate (e.g., by action of a mevalonate pyrophosphate decarboxylase (MVD) polypeptide); and (g) converting isopentenyl pyrophosphate (IPP) to dimethylallyl pyrophosphate (DMAPP) (e.g., by action of an isopentenyl pyrophosphate isomerase (IDI) polypeptide). A geranyl diphosphate synthase (GPPS) polypeptide then acts on IPP and/or DMAPP to generate GPP. Additionally, polypeptides that generate GPP or are part of a biosynthetic pathway that generates GPP may be one or more polypeptides having at least one activity of a polypeptide present in the deoxyxylulose-5-phosphate (DXP) pathway, instead of those of the MVA pathway.

In addition to the MVA pathway located in the cytosol, ER and peroxisomes, these intermediates can also arise from the more recently identified methylerythritol 4-phosphate (MEP) pathway localized in plastids (Hemmerlin et al., 2012).

Hexanoyl-CoA may be generated by polypeptides that generate acyl-CoA compounds or acyl-CoA compound derivatives (e.g., a hexanoyl-CoA synthase (HCS) polypeptide, an acyl-activating enzyme polypeptide, a fatty acyl-CoA synthetase polypeptide, or a fatty acyl-CoA ligase polypeptide). Hexanoyl-CoA may also be generated through pathways comprising one or more polypeptides that generate malonylCoA, such as an acetyl-CoA carboxylase (ACC) polypeptide. Additionally, hexanoyl-CoA may be generated by polypeptides that are part of a biosynthetic pathway that produces hexanoyl-CoA, including, but not limited to: a malonyl CoA-acyl carrier protein transacylase (MCTl) polypeptide, a PaaHl polypeptide, a Crt polypeptide, a Ter polypeptide, and a BktB polypeptide; a MCTl polypeptide, a PhaB polypeptide, a PhaJ polypeptide, a Ter polypeptide, and a BktB polypeptide; a short chain fatty acyl-CoA thioesterase (SCFA-TE) polypeptide; or a fatty acid synthase (FAS) polypeptide. Hexanoyl CoA derivatives, acyl-CoA compounds, or acyl-CoA compound derivatives may also be formed via such pathways and polypeptides.

Malonyl-CoA-intermediate is involved in a number of metabolic processes; it is a substrate in acylation and condensation reactions and as such it is found in plastids, the cytosol and mitochondria. It is made by carboxylation of acetyl-CoA in the plastid and cytosol and primarily produced by cytosolic ATP-dependent acetyl-CoA carboxylase (ACCase) malonyl-CoA synthetase (Guan et al (2016)).

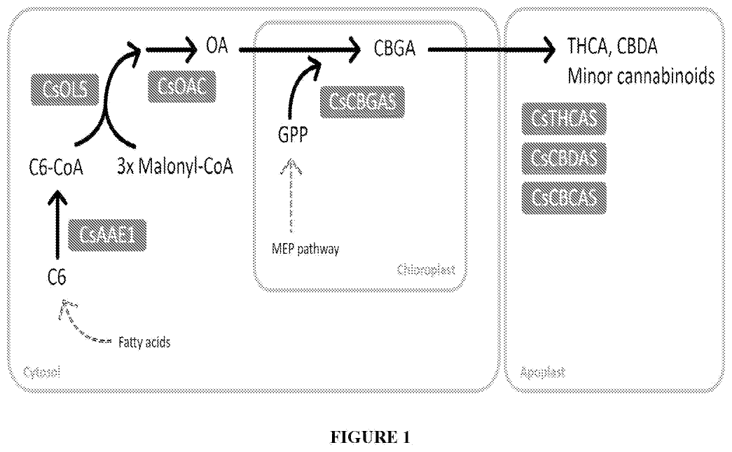

GPP and hexanoyl-CoA may also be generated through pathways comprising polypeptides that condense two molecules of acetyl-CoA to generate acetoacetyl-CoA and pyruvate dehydrogenase complex polypeptides that generate acetyl-CoA from pyruvate. Hexanoyl CoA derivatives, acyl-CoA compounds, or acyl-CoA compound derivatives may also be formed via such pathways. An exemplary cannabinoid biosynthetic pathway from Cannabis sativa is shown in FIG. 1.

The term “cannabinoid” relates to a group of closely related compounds which include cannabinol and the active constituents of Cannabis and may include cannabinoid derivatives, cannabinoid precursors, or cannabinoid precursor derivatives. A cannabinoid may be in an acid form or a non-acid form, the latter also being referred to as the decarboxylated form since the non-acid form can be generated by decarboxylating the acid form.

Cannabinoids may include, but are not limited to, cannabichromene (CBC) type (e.g. cannabichromenic acid), cannabigerol (CBG) type (e.g. cannabigerolic acid), cannabidiol (CBD) type (e.g. cannabidiolic acid), Δ9-trans-tetrahydrocannabinol (Δ9-THC) type (e.g. Δ9 tetrahydrocannabinolic acid), Δ8-trans-tetrahydrocannabinol (Δ8-THC) type, cannabicyclol (CBL) type, cannabielsoin (CBE) type, cannabinol (CBN) type, cannabinodiol (CBND) type, cannabitriol (CBT) type, cannabigerolic acid (CBGA), cannabigerolic acid monomethylether (CBGAM), cannabigerol (CBG), cannabigerol monomethylether (CBGM), cannabigerovarinic acid (CBGV A), cannabigerovarin (CBGV), cannabichromenic acid (CBCA), cannabichromene (CBC), cannabichromevarinic acid (CBCV A), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDV A), cannabidivarin (CBDV), cannabidiorcol (CBD-C1), Δ9-tetrahydrocannabinolic acid A (THCA-A), Δ9-tetrahydrocannabinolic acid B (THCA-B), Δ9-tetrahydrocannabinol (THC), Δ9-tetrahydrocannabinolic acid-C4 (THCA-C4), Δ9-tetrahydrocannabinol-C4 (THC-C4), Δ9-tetrahydrocannabivarinic acid (THCVA), Δ9-tetrahydrocannabivarin (THCV), Δ9-tetrahydrocannabiorcolic acid (THCA-C1), Δ9-tetrahydrocannabiorcol (THC-C1), Δ7-cis-iso-tetrahydrocannabivarin, Δ8-tetrahydrocannabinolic acid (Δ8-THCA), Δ8-tetrahydrocannabinol (Δ8-THC), cannabicyclolic acid (CBLA), cannabicyclol (CBL), cannabicyclovarin (CBLV), cannabielsoic acid A (CBEA-A), cannabielsoic acid B (CBEAB), cannabielsoin (CBE), cannabielsoinic acid, cannabicitranic acid, cannabinolic acid (CBNA), cannabinol (CBN), cannabinol methylether (CBNM), cannabinol-C4, (CBN-C4), cannabivarin (CBV), cannabinol-C2 (CNB-C2), cannabiorcol (CBN-C1), cannabinodiol (CBND), cannabinodivarin (CBVD), cannabitriol (CBT), 10-ethyoxy-9-hydroxy-delta-6atetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), Δ9-cistetrahydrocannabinol (cis-THC), 3,4,5,6-tetrahydro-7-hydroxy-alpha-alpha-2-trimethyl-9-npropyl-2,6-methano-2H-l-benzoxocin-5-methanol (OH-iso-HHCV), cannabiripsol (CBR), and trihydroxy-delta-9-tetrahydrocannabinol (triOH-THC).

In an embodiment, the present invention relates to the production of OA and/or CBGA which may be processed by a suitable method described herein or known in the art into any of the cannabinoids described above, preferably CBG, THC, CBD and CBN, most preferably CBG and CBD.

As disclosed herein, increased cannabinoid production produced by a genetically engineered plant, alga or plant cell can be the result of increasing expression of one or more enzymes associated with the cannabinoid biosynthetic pathway. Increased expression of an enzyme in a plant, alga or plant cell can include, for example, the introduction of a polynucleotide sequence encoding the enzyme into the plant, alga, part thereof, plastid or plant cell. Increasing expression can be achieved by increasing the copy number of one, two or all of the genes in a pathway thereby inducing a gene dosage effect as seen in plants expressing endogenous biosynthetic pathways introduction of additional copies of a gene contribute to greater accumulation of a desired product content in the plant. One example relates to increasing expression of a polynucleotide sequence encoding a polypeptide described herein in Cannabis satvia by inducing a gene dosage effect sufficient such that the expression results in a greater accumulation of, for instance, CBGA or OA in the plant. In an embodiment, introduction of a polynucleotide sequence encoding an enzyme can be accomplished by transformation or other methods known in the art or described herein. The polynucleotide may be introduced under the control of an inducible or developmental triggered promoter or a constitutitvely expressed promoter that targets a plant part for example targeting trichome, leaf or stem expression. The polynucleotide sequence may be introduced into a nuclear genome, plastid genome or be present in a vector such as a geminivirus vector or plasmid including geminivirus sequences or replication machinery. Examples of suitable cannabinoid biosynthetic pathway enzymes include, but are not limited to hexanoyl-CoA synthetase, acetyl-CoA carboxylase, MVA type III polyketide synthase (e.g., tetraketide synthase (TKS), Steely1 and Steely2), OAC, geranyl pyrophosphate synthase (GPPS), aromatic prenyltransferase, geranyl pyrophosphate:olivetolic acid geranyltransferase, cannabichromene synthase, tetrahydrocannabinolic acid synthase, and cannabidiolic acid synthase.

In addition to the exemplary cannabinoid biosynthetic pathway from Cannabis sativa shown in FIG. 1, alternative biosynthetic intermediates can be used in a cannabinoid biosynthetic pathway in a genetically engineered plant or plant cell. For example, olivetol is an intermediate that lacks the carboxyl group of olivetolic acid. Use of olivetol instead of olivetolic acid in a cannabinoid biosynthetic pathway will produce cannabinoids that similarly lack a carboxyl group such as cannabigerol (CBG), tetrahydrocannabinol (THC), or cannabidiol (CBD). In another example, tetraketide synthase (TKS) condenses butyryl-CoA and malonyl-CoA to form the intermediate trioxodecanoyl-CoA, and olivetolic acid cyclase (OAC) catalyzes an intramolecular aldol condensation of trioxodecanoyl-CoA to yield divarinolic acid. Divarinolic acid is an intermediate containing an n-propyl group in place of the n-pentyl group found in olivetolic acid. Use of divarinolic acid instead of olivetolic acid in a cannabinoid biosynthetic pathway will produce cannabinoids that similarly contain an n-propyl group such as cannabigerovarinic acid (CBGVA), tetrahydrocannabivarinic acid (THCVA), cannabidivarinic acid (CBDVA), or cannabichromevarinic acid (CBCVA). In another example, divarinol is an intermediate that lacks the carboxyl group of divarinolic acid, and contains an n-propyl group in place of the n-pentyl group found in olivetol. Use of divarinol instead of divarinolic acid in a cannabinoid biosynthetic pathway will produce cannabinoids that similarly contain an n-propyl group and lack a carboxyl group such as cannabigerovarin (CBGV), tetrahydrocannabivarin (THCV), cannabidivarinic acid (CBDV), or cannabichromevarinic acid (CBCV). A skilled person will understand that these alternative biosynthetic intermediates can be used in accordance with the invention for the production of OA and/or CBGA or other cannabinoids.

In addition to the exemplary cannabinoid biosynthetic pathway from Cannabis sativa shown in FIG. 1, alternative enzymes can be used in a cannabinoid biosynthetic pathway in a genetically engineered plant or plant cell. For example, in addition to the enzymes found in Cannabis sativa, alternative enzymes of a cannabinoid biosynthetic pathway may be found in other plants (e.g., Humulus lupulus, Rhododendron sp. such as Rhododendron dauricum or Rhododendron adamsii or the liverworts Radula sp such as Radula perrottetlii or Radua arginateata), in bacteria (e.g., Streptomyces), or in protists (e.g., Dictyostelium discoideum). It will be understood that enzymes that differ in structure, but perform the equivalent function may be used interchangeably in a cannabinoid biosynthetic pathway in a genetically engineered plant or plant cell.

In addition to the wild-type enzymes found in organisms discussed herein (e.g., in Cannabis sativa), modified variants of these enzymes can be used in a cannabinoid biosynthetic pathway in a genetically engineered plant or plant cell. Variants of enzymes for use in a cannabinoid biosynthetic pathway can be generated by altering the polynucleotide sequence encoding said enzyme to, for example, increase/decrease the activity of a domain, add/remove a domain, add/remove a signalling sequences, or to otherwise alter the activity, abundance or specificity of the enzyme.

Exemplary enzymes and/or products of the cannabinoid biosynthetic pathway include but are not limited to the following.

C. sativa acyl activating enzyme (CsAAE1) is a cytosolic CsAAE1 enzyme present in the trichome. It is a trichome-specific acyl-CoA synthetase that converts C6 in its CoA derivative. CsAAE1 is a member of the acyl-activating enzyme (AAE) superfamily that activate carboxylic acids through an adenylate intermediate (Stout et al., 2012). This family of enzymes all possess a well-conserved 12 amino acid residue AMP-binding motif (PROSITE PS00455) (Shockey and Browse, 2011). The AAE1 belongs to the EC6.2.1.2 classification and CsAAE1 is known to accept FA that have different chain lengths (Stout et al., 2012). An exemplary sequence to be used in accordance with the invention is AFN42527.1 or SEQ ID NO:1.

C. sativa olivetol synthase (CsOLS) is one of two enzymes working in concert to produce OA from C6-CoA and malonyl-CoA. Taura et al. (2009) identified the CsOLS gene as the polyketide synthase in C. sativa trichomes that condenses C6-CoA and a variety of other short-chain fatty acids together with malonyl-CoA. CsOLS can accept C4-C8 CoA substrates (Taura et al., 2009). CsOLS is a cytosolic protein (Stout et al., 2012) and its crystal structure has been resolved recently (Kearsey et al., 2020). Non-natural variant OLS mutants have been recently described in WO2020/214951. An exemplary sequence to be used in accordance with the invention is BAG14339.1 or SEQ ID NO:2.

C. sativa olivetolic acid cyclase (CsOAC) is the second enzymatic step needed to produce OA. CsOAC is localized in the cytosol (Gagne et al., 2012) and refers to a 3,5,7-trioxododecanoyl-CoA cyclase or a 3,5,7-trioxundecanoyl-CoA cyclase (EC4.4.1.26). This enzyme class is capable of converting 3,5,7-trioxododecanoyl-CoA into OA or 3,5,7-trioxundecanoyl-CoA into divarinolic acid. An exemplary sequence to be used in accordance with the invention is AFN42527.1 or SEQ ID NO:3.

C. sativa CBGA synthase (CBGAS). In Cannabis OA is transported into the chloroplast for further conversion to CBGA by the CsCBGAS (EC 2.5.1.102). Recently, both Luo et al. (2019) and Gulck et al. (2020) identified CsPT4 as the functional CBGAS prenyltransferase in C. sativa. Unlike CsOLS and CsOAC, CsCBGAS is a membrane-bound enzyme residing in the chloroplast (Gulck et al., 2020). An alternative prenyltransferase NphB from Streptomyces sp. strain CL190, capable of producing CBGA was identified by Valliere et al. (2019). This group used structure-based protein design to improve the non-specific CBGAS activity of the soluble NphB. CBGAS is capable of converting GPP and olivetolic acid (OA) or GPP and divarinolic acid (DVA) into cannabigerolicacid (CBGA) or cannabigerovarinic acid (CBGVA). An exemplary sequence to be used in accordance with the invention is DAC76710.1 or SEQ ID NO:4.

C. sativa THCA and CBDA synthases (THCAS; CBDAS). The final step in the cannabinoid biosynthesis pathway consists of the conversion of CBGA into THC or CBD as the major cannabinoid end-products by the CsTHCAS or CsCBDAS, respectively. In C. sativa, both enzymes are located in the apoplast, explaining the accumulation of the major cannabinoids such as THC and CBD at very high levels in the extra cellular storage globules (Sirikantaramas et al., 2004; Taura et al., 2007). CBDA synthase (EC1.21.3.8) is capable of converting CBGA or CBGVA into (CBDA) or CBDVA. CBDAS can accept CBGA (C5 tail) and CBGVA (C3 tail) in vitro (Valliere et al., 2019). THCA synthase (EC1.21.3.7) is able to convert CBGA or CBGVA into THCA or THCVA.

### Polypeptides

The terms “peptide,” “polypeptide,” and “protein” may be used interchangeably herein, and may refer to a polymeric form of amino acids of any length, which can include coded and non-coded amino acids, chemically or biochemically modified or derivatized amino acids, full-length polypeptides, fragments of polypeptides, or polypeptides having modified peptide backbones. The polypeptides disclosed herein may be presented as modified or engineered forms, including truncated or fusion forms, retaining the recited activities. The polypeptides disclosed herein may also be variants differing from a specifically recited “reference” polypeptide (e.g., a wild-type polypeptide) by amino acid insertions, deletions, mutations, and/or substitutions, but retains an activity that is substantially similar to the reference polypeptide.

Genetically engineered plants, algae, plastids or parts thereof of the invention may comprise an exogenous polynucleotide encoding a polypeptide as defined herein. In these instances, the plants and cells produce a recombinant polypeptide. The term “recombinant” in the context of a polypeptide refers to the polypeptide encoded by an exogenous polynucleotide when produced by a cell, which polynucleotide has been introduced into the cell or a progenitor cell by recombinant DNA or RNA techniques such as, for example, transformation. Typically, the cell comprises a non-endogenous gene that causes an altered amount of the polypeptide to be produced. In an embodiment, a “recombinant polypeptide” is a polypeptide made by the expression of an exogenous (recombinant) polynucleotide in a plant or algal cell.

In an embodiment, the polypeptides of the invention may be active in the cytosol and/or plastid of the plant. In this context, the term “active” will be understood to mean that the polypeptide is capable of exerting its normal biological activity, for example by catalysing the synthesis of olivetolic acid or CBGA, whether in the cytosol and/or plastid of the plant.

The % identity of a polypeptide is determined by GAP (Needleman and Wunsch, 1970) analysis (GCG program) with a gap creation penalty=5, and a gap extension penalty=0.3. The query sequence is at least 100 amino acids in length, and the GAP analysis aligns the two sequences over a region of at least 100 amino acids. More preferably, the query sequence is at least 300 amino acids in length and the GAP analysis aligns the two sequences over a region of at least 300 amino acids. Alternatively, the query sequence is at least 500 amino acids in length and the GAP analysis aligns the two sequences over a region of at least 500 amino acids. Even more preferably, the GAP analysis aligns two sequences over their entire length of any amino acid sequence disclosed herein including those defined in SEQ ID NOs: 1-4 and 20-23.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is preferably at least 50%, at least 60%, at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In an embodiment, a polypeptide, in some cases a fusion polypeptide, for use in, and/or of, the invention comprises a “chloroplast transporting peptide” (CTP), also known as a “chloroplast transit peptide”. Examples of suitable CTP's include any signal segment capable of directing the secretion or localisation of a polypeptide defined herein into a plastid such as a chloroplast. Non-limiting examples of plastid transporters include those described in AT1G61800, AT5G16150, AT5G33320, AT5G46110, AT4G15530, AT2G36580, AT3G52990, AT3G55650, AT3G55810, AT4G26390, AT5G08570, AT5G56350, AT5G63680, AT1G32440, AT3G22960, AT3G49160 and AT5G52920.

In an embodiment, the CTP is a Rubisco small subunit (Hirakawa and Ishida, 2010). In a preferred embodiment, the CTP is a Rubisco small subunit with an amino acid sequence at least 70%, at least 80%, at least 90%, at least 95%, at least 96%, at least 97%, at least 98%, at least 99% or 100% identical to the nucleotide sequence as set forth in SEQ ID NO:15.

It will be understood that the use of a particular plastid targeting peptide depends on where in the chloroplast the protein is to be targeted, eg stroma/inner membrane/outer membrane/thylakoid membrane and whether the enzymes are membrane bound or not. Suitable plastid targeting peptides may include any of those disclosed in the art including in Dugdale et al., 2013; Dugdale et al., 2014; Lee etl al., 2008; Jarvis 2008 and Bruce 2000. For the cannabinoid pathway, the biosynthetic genes of the cannabinoid pathway are generally soluble and therefore may be targeted to the stroma using Rubisco targeting peptides. Predicted sequence motifs of suitable plastid targeting peptides may include any of the following outlined in Table 1.

TABLE 1
Predicted sequence motifs

LKETT FSNHL IKEQR
MAYSA FLHQS IICKA QSHED
AMKLP FPNAI TDSES SLGHR VNGSK LDSAA
CRLFR CNLLP LPKPL NRIAS PNGVP
EERKK YQNGD DVYAD LDWDN YMYVM CSKDG
AFATG PTFLG KISNT SVVSC
RQQTP QSLFG VTAMA KFITP
MITLR NLSRN SHQST RSFHN STGGF SSSND
RRLLQ NLPAL QQIPL SRGFI
FSSSC FLHRR KLTAI EQTTL DNRFS GSDSD
EALTT LFANF GL VSI LSTLG
ASTIK ATVRI TAAVA GVAAV VLLGG
VPIMS GISSR RFSIG FGFRF DVPFK RLRFV
EESSM DFVAT KFPAE LRYFP
MAAST SSPAF LSPAA LGSGR TMRKT
KAVKL PAASE GPFSG PGDYG
YSTQA QPQTN LITCA LAADS KSTFM GAAKP
VYPLA GLVSF KKLFL RAMSE DKESS
MAAAT GRLSL AQASF FVLQL
NAAVF SQWRP EDPRS KAPPY KGKFL DVNQA
HFLRN QPKVH VPLAL PKKVE PKSKV
FATIA SAAVK GGSSL GAKLF GDKSV EDLGN
LQARP LAIAA SFSNG LSFNL RQLPT LTVSC
SLQAR PRQLV QVKSF LRQLP VRKQL
TFTAT SMVAP SSSSF RRQSV RRSSS IVCAA
NSISF RPLRL VVRAE SIVIS LSLFL
AAVTR ARRLG SAVVD ARGVI
VGTSF LRSLP NTQSL KVKFI ELEVE VLDAA
QLLLS HSQIT PATKI VWRPK SSAVD PTKTL
AQATM FNGLK TRKAN RVNCM GKKKF LTDSE
MAAAM SLNPS QTKSK LAVTS SLSYS
MAAAM SPALS SSSSF RLRVS ASSVT
ATVTI LKLAV TLSTI PPKLA VIAVA ASIVL
HVGMK MLQIA SSVFF AISPA
AGFTV TAARG SGRKG
AVIQT GLLSL LSFNG FQTFE GISIS FICKA
FSLTF SVLVA AAQTT LKDGF
LNHKS KGSFF TCKAK SEASV PLGIN
MAATA FPALG KISNF IHCMS KAYKR
NFANL PINPG FLFKS TVQAR AEKTV
MASAN SSASV SSRQS KRTIR FSVRA VKEIA
MAATN GVSLN HRLQS FAVKA VVSAA KGTSD
RVLVS LRHSG SNNNK TTVRF SLNEI GLDSS
PPPPL QLSND
RPALL PMGKK RCSME AVMSN PAMAL
SAVIG SGCAQ LRSKF SIRME ESSKT
INRAP FLGKK SFKVL VKEDK AYDTS
SGIFG QARFG PAPPP NPPEW
RKSLS RCSVE TVTER LLLRA VDRPP
SACYI KSNNL PLAVE KELRS FVQTA VTGSL
LFSCC NISQK SHCPI SQSQE PLRSV SLFAL
IDSRA RLASA PSCIS TLPIQ EAPAT TTEAE
RIKLS ESLPI AICAA SSVEE
GTRVV DISSN KKAKT FPGAK DPFGL
SIGSC FKAPN VLISA
MDTLL NKLDF FHGFE SNNPY VKKRA GSAAL
LVLSE RPLPR IYTTP NFLSN SFGLN PLTTP
MASVA LISSP PSVSF RSSLS STSRR SFAVK
ETLIP MTTGT ALSFL LQLMR
TKPKP NTATP THHQK PPSLV VAFSE RDVTT
VAAVK GGSSL ANGVV GDKSV
GASVF SEFPI ESSKI LKKNA TRNVG AMQAG
GFSKS FGISS GPGSS ENPRP
QFHRS SLLGS LGISS GSDFS NLTAS IENPR
AMSLE LSRNH SSLLG RSFQN DFSSR NLNVT
VTFSV FTEFS GSSGG NGFGR
ASTSL FLSPS SSRLP IKCSK TETSS
ASIFR TGSSG FTSIG GSLAG
RAAIL ASIKL SNGGI RCNAV VTELA
LQPAK KSFGL TCSLH AGFAL SALVV
QPSIL FSNNG KRAPT CMAVA ETKTA
ASLKL ISKVG GLSFL RLIVT VVAPW AESKK
RRFST PPPPT KEIQE
PLCDS SQFVI NRLLS SSFWG GVSGC ASLGG
LCSST TTLSS QTSSS RRELS SSLVA SNDRR
GIFNS SGVKK FSTTH AIRAE
SNLTT VSSKP SSLSF RASSV
AGLRG RVSVG RTGLV RSVIG
ASLGV MLGTP LNFRA FSKKK TSDEL DGLLD
SPMAS SASLS PKGIS VSPTK FTVRA SDKTT
AFAFP SYIIT KGAST HLLFS VAINC AVGRR
MASLL FGSKL KNRSR MNVAT EINST GKFDS
KINGS LNGSG VAQRS AQQSE RSVIG VAAGL
AFSVR FKDSS ASITD QIKSE RNLAI
MDVAS HPSYY GKPIC SLIRI FMGET
RHAPR STDPK STQSA SVKWS PDQKD
AAAAA GIHPG PLWYP GSLVG
MASNA LSSFT NPALS SASPS FSRKV VAAAT
VLTRQ SSRVN CSLGS VSAIG TAIIR
ASVYA FPIQN VSFRL KLRFL VKAQS GASSN
LNPDP RVLES NQRLN QIPTW
PLFLL LYISR RAFPP PLVSL LIPTF NHQVR
AQATM TRKAN VSCMK IGKKK
AQATM TRKAN VSCMK KKFET
DTVFL FAGAK HIRTK NSRFH SIIAS AGTDS
QFTRN PFFSV IACAA CACTL
FSVRK FKESS SLSEQ SKADF SSSLR KAIIR
APRKG SIFAT AVRSN GDNAV
SVLTC GSGSV VGLVN VGFGQ KQMIM QRVVG
MTIAL FSGLP SSFSS SFRLK VVCSS SSSVM
RSIGS SVNHS NVPRS PMKPV PALQF
SSSSS LNSGS NLSSL ASPTS PILGV NKDPS
MAVCN FLQAP SRRAT TLSAG YGRLK TVTFC
RLLSA HVTSI LSNRT NLRRF
WTPLC PPERN PHCSP FRTAL PHLVA
PFSSH PNPST ETERP DDVTP
FNGLR PMRRK IMVTS TTLML RFGLA ANRKA
AMLQT TSPTF SYRSQ RVNRS SSSTG VKEKS
NVATM TGYSG YAPSF SIKCV SDTKK STNLI
VIAVA IVSGI LSGIL LTGVR FSSIG DAQSS
SAVKK HLFSS APAVK PQLDP NTPSP
FLFSR KSLVR SELPE KSVAE PAVVP
FLQTN LEIIP SLTDH KKRYN LQKAG GLEFD
LGSTK VQISL QTRKN FQIQA
SFISQ AAPET SDNTP QTIKV
FILTA GGGSS TTIVS DTPPA AKVPA
FSGLR SPKLD FHRVN VVAMA
LVNFH DVSPL ISSNL SSGKS STTDA
LQSPG VSRLL TIFSV CISKG YSPLL
LHKAS ALSLL VQAAK KSLTG EVKKE TPFVS
ASMTM SLEVK RDLMF VCSLA EPKRG
KSQSK KIPFL HNPLR VAAPP VPTSD EEKRI
FSLLN DISPL SSGKS TLSGV PFEEV
LSPAT ALMAM SGLFV TKMNH ISCQA LGALS
MAMTT PANVT ASSSR SSVSF VVRAA ASSSS
FELSR PVSPL RSLVP TPNSE PVYVP
GLAIR PPSDG IPADS HPISA TADFP
MAMQS PVPKL DKNFF YCKSK
FLQTN SLTDH KKRYN QKAGS
FRTAS KPIAH IPARH SCINP YPPSN
YKSNL LTLDS RCCKI LLVAT EEGDY
ASSFT PISLN FLLKK AVSPI
NSLMS AVYPS LLSSS KSKFV IRMAA
AQATM TRKTN VSCMK KKKFE DLSDV
ETALL VNFSG MASGL TALAF LAVVK QGLLA
ASPVL LKPNA KNRVS RIVVK AAASG ESKEE
ATKIF VISAT GTAAS AANYG YGGNS
LQLIL PPRPR QELSS TSPPI LPKLI PLTTP
VSAIF VTVAA LRQFQ IECSS
RSYLT SVKPL SVQVT RHLLS SGARR
MATCI SRFCC NFFRP SVNRK KSDVK ASITS
LSLKL PLFPF ISSEF SPDAD
ERRLD EQTKE LEKVE VSAYD WVAMV LFPQC
HTARH PSRLF RFSDR RSLLS VRDSR QRSWE
STATM KGQSL LTVRA LVKTA SNATC
NPTSH WLKPP HSLPI
LSLRL PTTGG CRIPY SGVPT CSLDS DSSVV
ATTVH SLFIQ LTSQG FDFKL YMIRK AESVN
ALLSD RSPPP PLTTP SLVEV SDPPP SSGSG
NPKSL PLCVS VLPPS PAEYP FVRNV VSSDF
GFPVI DQNPS KRRHI SGSSQ EKISP
LTISG PLNVA KSRSG IHCSK LPLIH
AFTCT FAVLP LTSIH VVCEA QETTT
ATVPL QFPCK IGVSV FKIRA GQDGV ASSST
QPTSD KSPKG HPKPE NPTRP DVGID SGTST
PQLLL QFRFT NYVAS CQRAS VIEGL
STDSC TDLPS NNSHS YSNRP
ITTMN AISDT LRGQF QRIHF LQHTR FGHVS
AAAMS KISTG SSYGD
MASLM GSTSL NKDKL KLGTS FLKAK TMTVA
MTKLL LCFAK GYCSN TCYAT
TNSFL NTQIP TTQVQ SQERV FAAGP
GQSVL LTVRA IASPG SNATC
LSLIC NTPNK CARVG EIPAN RARQI
SMISI FVISS SFSRK VSTLP SVPVV
HSRSP LSAAM EKSRQ SKVTV KLIAS NALKL
GSETP PIVGP
SRTPP QAIDP PCNPL LSRFL ATPDP
EFTVN NLRSL LRHGF SSQSW LKLAR LAMYS
TPHTI RLRSL SILPD GDDFI VFEDP
CLLPQ CPPDS FFQPQ SFDTD DSLIS
RTYIF SPLCL PRGDS LRPRV ESYGS LLRRP
QSQFL ANNRV CQITR SFKVE KFQLQ VKNGI
MGVSL TSHNN LRHLS LLQRK FQHSV
AVIGL KKASN
TELNF HLLPI LLSFT SIRME ATGAG
KSKKE KKRGV IRNHR TLPEL
YAIPR SFVKM SMGIK KLALA VTCMA MEVEA
HLNLR SRIGN KNLRN KLPPD RKPKI
KLTVK SRQSD EKQRF GDSSS SQNAE
SKIKL TLLLS FTRLC KAMDS CCISN
EDEYI DYEKE GLEDK EEVME EQMGA VLILM
MAYLA SMTMN TTAAE KOKKR ESKGF EEMRF
PSENV RFSVA RKVLA PGKLE REPVE
FQTKT SVVML ASVLS
REILR AYQAF DFDPS ERSGL RLTKN
VLTCA TKKLT TELSI RCSGN VLLEG
MNIVS AFGGK
MGSKQ GAYYG ILSLI VAVIL ILWLI
ASSSL SLRPR SFINP SLGRR AAKKK
SRPRI TNSML PHPLI LLRSD IPMTS
SSSTF LTTTS VSRKS KTTST
SMAMC TGTGK RLSVD IATRF RAEII SDKIQ
SSFTI MGLKL LKVTC ASTMT
MTSMI VKTTP SRSNR RFPRT RLSIA LSPVS
FSQAS NRTLC STPRN REAFC SCRFH NGAPS
MELTL SSSSL AKSSR QESSS AKMKP KTFTP
TQESA SRNRS SNALY FNGFR LSFNT
LCSAS FHALK SRKKH SMRCS SMKSS KSSKP
TILTV FSSSR VSLKS STSSP KAGVS
GQAFS WSSAS SSGVF ERCGC
DVPFF KDKVV
VMALA TSKSA VSVTS HRIKS AERTK
ALQRL PRRPP PPPSS IVGYP ELKFA
LTSVA TTITT RLCFS RFRIA KSSVV
CLLPR SDSVN CKSKI SSVKR QSKHE EDPDD
LLSRT NPSPN NFGSQ YTINP SAIAS
ALNSS AFFVP LSSAR TRSTI SRRAF SPQKS

In a particular embodiment, the fusion protein or polypeptide for expression in a plant or alga comprises: i) a polypeptide selected from the group consisting of a polyketide synthase, a polyketide cyclase, an acyl-activating enzyme, a prenyltransferase, a plastid acyl-lipid thioesterase, a plastid lipase or a plastid tomato 13-lipoxygenase, and a plastid transporting peptide ii) wherein the polypeptide of (i) increases the production of cannabigerolic acid (CBGA) in the presence of hexanoic acid (C6) and geranyl-pyrophosphate (GPP), when compared to a wild type plant or alga.

It will be understood that polyketide synthases (PKSs) are a family of multi-domain enzymes or enzyme complexes that produce polyketides, a large class of secondary metabolites, with particular relevance in plants and may include Type I polyketide synthases, type II polyketide synthases and type III polyketide synthases such as olivetolic acid synthase (OAS). It will be understood that polyketide cyclases represent a number of cyclases involved in polyketide synthesis in a number of actinobacterial species and may include olivetolic acid cyclase (OAC).

### Acyl-Activating Enzymes

Acyl-activating enzymes are a diverse group of proteins that catalyze the activation of many different carboxylic acids, primarily through the formation of a thioester bond. This group of enzymes is found in all living organisms and includes the acyl-coenzyme A synthetases, 4-coumarate:coenzyme A ligases, luciferases, and non-ribosomal peptide synthetases. The members of this superfamily share little overall sequence identity, but do contain a 12-amino acid motif common to all enzymes that activate their acid substrates using ATP via an enzyme-bound adenylate intermediate. A non-limiting example of this enzyme is acyl activating enzyme (CsAAE1).

Prenyltransferases are a class of enzymes that transfer allylic prenyl groups to acceptor molecules. Prenyl transferases commonly refer to prenyl diphosphate syntheses. Prenyltransferases are commonly divided into two classes, cis and trans, depending upon the stereochemistry of the resulting products. A non-limiting example of this enzyme is cannabigerolic acid synthase (CBGAS).

A fusion protein may comprise a linker between the CTP and an enzyme CBD biosynetetic pathway described herein.

In an embodiment, the fusion protein may include an N-terminal fusion or C-terminal fusion. For instance, the N-terminal fusion may include a chloroplast transporting peptide and the C-termical fusion may include a reporter protein known in the art such as mNeonGreen or green fluorescent protein (GFP), red fluorescent protein (RFP), mCherry, luciferase enzyme, or β-galactosidase.

Amino acid sequence mutants of the polypeptides for use in the invention can be prepared by introducing appropriate nucleotide changes into a nucleic acid defined herein, or by in vitro synthesis of the desired polypeptide. Such mutants include, for example, deletions, insertions or substitutions of residues within the amino acid sequence. A combination of deletion, insertion and substitution can be made to arrive at the final construct, provided that the final peptide product possesses the desired characteristics. Preferred amino acid sequence mutants have one, two, three, four or less than 10 amino acid changes relative to the reference polypeptide such as comprising an amino acid provided in SEQ ID NOs:1 to 4.

Mutant (altered) polypeptides can be prepared using any technique known in the art, for example, using directed evolution, rational design strategies or mutagenesis (see below). Products derived from mutated/altered DNA can readily be screened using techniques described herein to determine if, when expressed in a plant cell produce a product defeined herein.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. Where it is desirable to maintain a certain activity it is preferable to make no, or only conservative substitutions, at amino acid positions which are highly conserved in the relevant protein family. Examples of conservative substitutions are shown in Table 2 under the heading of “exemplary substitutions”.

In an embodiment, a mutant/variant polypeptide has one or two or three or four conservative amino acid changes when compared to a naturally occurring polypeptide. Details of conservative amino acid changes are provided in Table 2. In a preferred embodiment, the changes are not in one or more of the motifs which are highly conserved between the different polypeptides of the same class of enzyme. As the skilled person would be aware, such minor changes can reasonably be predicted not to alter the activity of the polypeptide when expressed in a recombinant cell.

The primary amino acid sequence of a polypeptide can be used to design variants/mutants thereof based on comparisons with closely related polypeptides. As the skilled addressee will appreciate, residues highly conserved amongst closely related proteins are less likely to be able to be altered, especially with non-conservative substitutions, and activity maintained than less conserved residues (see above).

TABLE 2
Exemplary substitutions.

| Original Residue | Exemplary Substitutions |
|---|---|
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

### Directed Evolution

In directed evolution, random mutagenesis is applied to a protein, and a selection regime is used to pick out variants that have the desired qualities, for example, increased activity. Further rounds of mutation and selection are then applied. A typical directed evolution strategy involves three steps:

1) Diversification: The gene encoding the protein of interest is mutated and/or recombined at random to create a large library of gene variants. Variant gene libraries can be constructed through error prone PCR (see for example, Leung, 1989 Cadwell and Joyce, 1992), from pools of DNaseI digested fragments prepared from parental templates (Stemmer, 1994a; Stemmer, 1994b; Crameri et al., 1998: Coco et al., 2001) from degenerate oligonucleotides (Ness et al., 2002, Coco, 2002) or from mixtures of both, or even from undigested parental templates (Zhao et al, 1998; Eggert et al., 2005; Jéz\acute{q}uek et al., 2008) and are usually assembled through PCR. Libraries can also be made from parental sequences recombined in vivo or in vitro by either homologous or non-homologous recombination (Ostermeier et al., 1999; Volkov et al., 1999; Sieber et al., 2001). Variant gene libraries can also be constructed by sub-cloning a gene of interest into a suitable vector, transforming the vector into a “mutator” strain such as the E. coli XL-1 red (Stratagene) and propagating the transformed bacteria for a suitable number of generations. Variant gene libraries can also be constructed by subjecting the gene of interest to DNA shuffling (i.e., in vitro homologous recombination of pools of selected mutant genes by random fragmentation and reassembly) as broadly described by Harayama (1998).

2) Selection: The library is tested for the presence of mutants (variants) possessing the desired property using a screen or selection. Screens enable the identification and isolation of high-performing mutants by hand, while selections automatically eliminate all nonfunctional mutants. A screen may involve screening for the presence of known conserved amino acid motifs. Alternatively, or in addition, a screen may involve expressing the mutated polynucleotide in a host organism or part thereof and assaying the level of activity.

3) Amplification: The variants identified in the selection or screen are replicated many fold, enabling researchers to sequence their DNA in order to understand what mutations have occurred.

Together, these three steps are termed a “round” of directed evolution. Most experiments will entail more than one round. In these experiments, the “winners” of the previous round are diversified in the next round to create a new library. At the end of the experiment, all evolved protein or polynucleotide mutants are characterized using biochemical methods.

### Rational Design

A protein can be designed rationally, on the basis of known information about protein structure and folding. This can be accomplished by design from scratch (de novo design) or by redesign based on native scaffolds (see, for example, Hellinga, 1997; Lu and Berry, 2007). Protein design typically involves identifying sequences that fold into a given or target structure and can be accomplished using computer models. Computational protein design algorithms search the sequence-conformation space for sequences that are low in energy when folded to the target structure. Computational protein design algorithms use models of protein energetics to evaluate how mutations would affect a protein's structure and function. These energy functions typically include a combination of molecular mechanics, statistical (i.e. knowledge-based), and other empirical terms. Suitable available software includes IPRO (Interative Protein Redesign and Optimization), EGAD (A Genetic Algorithm for Protein Design), Rosetta Design, Sharpen, and Abalone.

### Polynucleotides and Genes

The present invention refers to various polynucleotides encoding polypeptides that increase the production of a cannabinoid, preferably CBGA. As used herein, a “polynucleotide” or “nucleic acid” or “nucleic acid molecule” means a polymer of nucleotides, which may be DNA or RNA or a combination thereof, and includes genomic DNA, mRNA, cRNA, and cDNA. Less preferred polynucleotides include tRNA, siRNA, shRNA and hpRNA. A given polynucleotide may be of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. Basepairing as used herein refers to standard basepairing between nucleotides, including G:U basepairs. “Complementary” means two polynucleotides are capable of basepairing (hybridizing) along part of their lengths, or along the full length of one or both. The term “polynucleotide” is used interchangeably herein with the term “nucleic acid”.

The polynucleotides or nucleic acid sequences of the present application may be deoxyribonucleic acid (DNA) sequences or ribonucleic acid (RNA) sequences and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences.

The term “nucleic acid molecule” or its derivatives, as used herein, is intended to include unmodified DNA or RNA or modified DNA or RNA For example, it may be useful for the nucleic acid molecules of the disclosure to be composed of single- and double-stranded DNA, DNA that is a mixture of single- and double-stranded regions, single- and double-stranded RNA, and RNA that is a mixture of single- and double stranded regions, hybrid molecules comprising DNA and RNA that may be single stranded or, more typically double-stranded or a mixture of single- and double-stranded regions. In addition, it may be useful for the nucleic acid molecules to be composed of triple stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” encompasses chemically, enzymatically, or metabolically modified forms. The term “polynucleotide” shall have a corresponding meaning. In some embodiments, the genetically engineered plant, part thereof, alga or plastid comprises at least one polynucleotide described herein.

The term “isolated polynucleotide” means a polynucleotide which has generally been separated from the polynucleotide sequences with which it is associated or linked in its native state, if the polynucleotide is found in nature. Preferably, the isolated polynucleotide is at least 90% free from other components with which it is naturally associated, if it is found in nature. Preferably the polynucleotide is not naturally occurring, for example by covalently joining two shorter polynucleotide sequences in a manner not found in nature (chimeric polynucleotide).

A genomic form or clone of a gene containing the transcribed region may be interrupted with non-coding sequences termed “introns” or “intervening regions” or “intervening sequences”, which may be either homologous or heterologous with respect to the “exons” of the gene. An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. As described herein, the barley CAD2 genes (both resistant and susceptible alleles) contain two introns in their protein coding regions. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above. A gene may be introduced into an appropriate vector for extrachromosomal maintenance in a cell or, preferably, for integration into the host genome.

As used herein, a “chimeric gene” refers to any gene that comprises covalently joined sequences that are not found joined in nature. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. The term “endogenous” is used herein to refer to a substance that is normally present or produced in an unmodified plant at the same developmental stage as the plant under investigation. An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA/RNA technology. The terms “foreign polynucleotide” or “exogenous polynucleotide” or “heterologous polynucleotide” and the like refer to any nucleic acid which is introduced into the genome of a cell by experimental manipulations.

Foreign or exogenous genes may be genes that are inserted into a non-native organism or cell, native genes introduced into a new location within the native host, or chimeric genes. Alternatively, foreign or exogenous genes may be the result of editing the genome of the organism or cell, or progeny derived therefrom. A “transgene” is a gene that has been introduced into the genome by a transformation procedure.

The term “genetically engineered”, “genetically modified”, “genetic modification” or variants thereof refers to any genetic manipulation by man and includes introducing genes into cells by transformation or transduction, gene editing, cisgenesis, mutating genes in cells and altering or modulating the regulation of a gene in a cell or organisms to which these acts have been done or their progeny and so on.

Furthermore, the term “exogenous” in the context of a polynucleotide (nucleic acid) refers to the polynucleotide when present in a cell that does not naturally comprise the polynucleotide. The cell may be a cell which comprises a non-endogenous polynucleotide resulting in an altered amount of production of the encoded polypeptide, for example an exogenous polynucleotide which increases the expression of an endogenous polypeptide, or a cell which in its native state does not produce the polypeptide. Alternatively, the cell may be a cell with an altered chromosome position. Increased production of a polypeptide of the invention is also referred to herein as “over-expression”. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically, such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3. The query sequence is at least 900 nucleotides in length, and the GAP analysis aligns the two sequences over a region of at least 900 nucleotides. Preferably, the query sequence is at least 975 nucleotides in length, and the GAP analysis aligns the two sequences over a region of at least 975 nucleotides. Even more preferably, the query sequence is at least 1,050 nucleotides in length and the GAP analysis aligns the two sequences over a region of at least 1,050 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 50%, at least 60%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

When referring to polynucleotide identity herein, it will be understood that a given sequence identity is in reference to the open reading frame sequence.

In a further embodiment, the present invention relates to polynucleotides which are substantially identical or identical to those specifically described herein. As used herein, with reference to a polynucleotide the term “substantially identical” means the substitution of one or a few (for example 2, 3, or 4) nucleotides whilst maintaining at least one activity of the native protein encoded by the polynucleotide. In addition, this term includes the addition or deletion of nucleotides which results in the increase or decrease in size of the encoded native protein by one or a few (for example 2, 3, or 4) amino acids whilst maintaining at least one activity of the native protein encoded by the polynucleotide.

The present invention also relates to the use of oligonucleotides, for instance in methods of screening for a polynucleotide of, or encoding a polypeptide of, the invention. As used herein, “oligonucleotides” are polynucleotides up to 50 nucleotides in length. The minimum size of such oligonucleotides is the size required for the formation of a stable hybrid between an oligonucleotide and a complementary sequence on a nucleic acid molecule of the present invention. They can be RNA, DNA, or combinations or derivatives of either. Oligonucleotides are typically relatively short single stranded molecules of 10 to 30 nucleotides, commonly 15-25 nucleotides in length. When used as a guide for genome editing, probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, more preferably at least 22 nucleotides, even more preferably at least 25 nucleotides in length. Oligonucleotides of the present invention used as a probe are typically conjugated with a label such as a radioisotope, an enzyme, biotin, a fluorescent molecule or a chemiluminescent molecule.

As those skilled in the art would be aware, the sequence of the oligonucleotide primers described herein can be varied to some degree without effecting their usefulness for the methods of the invention. A “variant” of an oligonucleotide disclosed herein (also referred to herein as a “primer” or “probe” depending on its use) useful for the methods of the invention includes molecules of varying sizes of, and/or are capable of hybridising to the genome close to that of, the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise the target region. In addition, variants may readily be designed which hybridise close (for example, but not limited to, within 50 nucleotides or within 100 nucleotides) to the region of the genome where the specific oligonucleotides defined herein hybridise.

The present invention includes oligonucleotides that can be used as, for example, guides for RNA-guided endonucleases, probes to identify nucleic acid molecules, or primers to produce nucleic acid molecules. Probes and/or primers can be used to clone homologues of the polynucleotides of the invention from other species. Furthermore, hybridization techniques known in the art can also be used to screen genomic or cDNA libraries for such homologues.

Polynucleotides and oligonucleotides of the present invention include those which hybridize under stringent conditions to one or more of the sequences disclosed herein or in the art capable of increasing cannabinoid production. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid). A variant of a polynucleotide of the invention includes molecules of varying sizes when compared to the reference polynucleotides defined herein. For example, variants may comprise additional nucleotides (such as 1, 2, 3, 4, or more), or less nucleotides as long as they encode a functional protein. Furthermore, a few nucleotides may be substituted without influencing the integrity of the encoded protein. In addition, variants may include polynucleotides which encode the same polypeptide or amino acid sequence but which vary in nucleotide sequence by redundancy of the genetic code. The terms “polynucleotide variant” and “variant” also include naturally occurring allelic variants.

### Nucleic Acid Constructs

The present invention further relates to nucleic acid constructs comprising the polynucleotides of the invention, and vectors and host cells containing these, methods of their production and use, and uses thereof.

In order to enable expression of the polynucleotides in a plant, part thereof, alga or plastid thereof, the nucleic acid construct may comprise a coding region encoding a polynucleotide sequence described herein, operably linked to one or more regulatory sequences, such as a promoter sequence, optionally heterologous promoter sequence that functions in a plant, alga or plastid wherein the nucleic acid construct is to be expressed.

A first polynucleotide is operably linked with a second polynucleotide when the first polynucleotide is in a functional relationship with the second polynucleotide. When recombinantly produced, operably linked polynucleotides are generally contiguous, and, where necessary to join two protein-coding regions, in the same reading frame (e.g., in a polycistronic ORF or joined by a peptide linker). However, the polynucleotides need not be contiguous to be operably linked.

Regulatory sequences, or control elements, refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In preferred embodiments of the present invention, the cis-acting sequence is an activator sequence that enhances or stimulates the expression of an expressible genetic sequence.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a polynucleotide encoding a signal sequence which may be operably linked to a coding sequence for expression in a cell, preferably a plant or algal cell. A “plant promoter” may be a promoter capable of initiating transcription in plant cells. Similarly, an algal promoter may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma.

Promoters which initiate transcription only in certain tissues are referred to as “tissue-specific.” A “cell type-specific” promoter primarily drives expression in certain cell types in one or more organs, for example, trichomes or vascular cells in roots or leaves. Promoters which initiate transcription only in certain subcellular organelles (e.g., such as mitochondria or chloroplasts or other plastids) are referred to as “plastid-specific”, “chloroplast-specific”, or “mitochondrion-specific” promoters, respectively. It will be understood that some promoters may initiate transcription in more than one subcellular location.

In an embodiment, the nucleic acid construct contains a promoter or regulatory element that is operable in a plastid. Alternatively the nucleic acid construct contains a promoter or regulatory element that is operable in the nucleus or cytosol.

An “inducible” promoter may be a promoter which may be under environmental control. Examples of environmental conditions that may initiate transcription by inducible promoters include anaerobic conditions and the presence of light. Tissue-specific, tissue-preferred, cell type 25 specific, and inducible promoters constitute the class of “non-constitutive” promoters. A “constitutive” promoter is a promoter which may be active under most environmental conditions or in most cell or tissue types.

Any inducible promoter can be used in some embodiments of the invention (see Ward et al., 1993). With an inducible promoter, the rate of transcription increases in response to an inducing agent. Exemplary inducible promoters include, but are not limited to: promoters from the ACEI system that responds to copper; 1n2 gene from maize that responds to benzenesulfonamide herbicide safeners; Tet repressor from TnlO; and the inducible promoter from a steroid hormone gene, the transcriptional activity of which may be induced by a glucocorticosteroid hormone (Schena et al., 1991).

Exemplary constitutive promoters include, but are not limited to: promoters from plant viruses, such as the 35S promoter from cauliflower mosaic virus (CaMV); promoters from rice actin genes; ubiquitin promoters; pEMU; MAS; maize H3 histone promoter; and the ALS promoter, Xbal/Ncol fragment 5′ to the Brassica napus ALS3 structural gene (or a nucleotide sequence similarity to said Xbal Ncol fragment) (WO 96/30530).

Additionally, any tissue-specific promoter may be utilized in some embodiments of the invention. Plants transformed with a nucleic acid construct comprising a coding sequence operably linked to a tissue-specific promoter may produce the product of the coding sequence exclusively, or preferentially, in a specific tissue. Exemplary tissue-specific or tissue-preferred promoters include, but are not limited to: a root-preferred promoter, such as that from the phaseolin gene; a leaf-specific and light-induced promoter such as that from cab or rubisco; an anther-specific promoter such as that from LAT52; a pollen-specific promoter such as that from ZmJ3; and a microspore-preferred promoter such as the apg promoter.

Promoters suitable for use in nucleic acid constructs of the invention include those that are inducible, viral, synthetic, or constitutive, all of which are well known in the art.

For the purpose of expression in specific tissues of the plant such as the leaf, seed, root or stem, it is preferred that the promoters utilized in the present invention have relatively high expression in these specific tissues. For this purpose, one may choose from a number of promoters for genes with tissue- or cell-specific, or -enhanced expression. Examples of such promoters include, the chloroplast glutamine synthetase GS2 promoter from pea, the chloroplast fructose-1,6-biphosphatase promoter from wheat, the nuclear photosynthetic ST-LS1 promoter from potato, the serine/threonine kinase promoter and the glucoamylase (CHS) promoter from Arabidopsis thaliana. Also reported to be active in photosynthetically active tissues are the ribulose-1,5-bisphosphate carboxylase promoter from eastern larch (Larix laricina), the promoter for the Cab gene, Cab6, from pine, the promoter for the Cab-1 gene from wheat, the promoter for the Cab-1 gene from spinach, the promoter for the Cab 1R gene from rice, the pyruvate, orthophosphate dikinase (PPDK) promoter from Zea mays, the promoter for the tobacco Lhcb1*2 gene, the Arabidopsis thaliana Suc2 sucrose-H30 symporter promoter, and the promoter for the thylakoid membrane protein genes from spinach (PsaD, PsaF, PsaE, PC, FNR, AtpC, AtpD, Cab, RbcS). Other promoters for the chlorophyll u/0-binding proteins may also be utilized in the present invention such as the promoters for LhcB gene and PsbP gene from white mustard (Sinapis alba).

A variety of plant gene promoters that are regulated in response to environmental, hormonal, chemical, and/or developmental signals, also can be used for expression of RNA-binding protein genes in plant cells, including promoters regulated by (1) heat, (2) light (e.g., pea RbcS-3A promoter, maize RbcS promoter), (3) hormones such as abscisic acid, (4) wounding (e.g., WunI), or (5) chemicals such as methyl jasmonate, salicylic acid, steroid hormones, alcohol, Safeners (WO 97/06269), or it may also be advantageous to employ (6) organ-specific promoters.

Other non-limiting examples describing such promoters include U.S. Pat. No. 6,437,217 (maize RS81 promoter); U.S. Pat. No. 5,641,876 (rice actin promoter); U.S. Pat. No. 6,426,446 (maize RS324 promoter); U.S. Pat. No. 6,429,362 (maize PR-i promoter); U.S. Pat. No. 6,232,526 (maize A3 promoter); U.S. Pat. No. 6,177,611 (constitutive maize promoters); U.S. Pat. Nos. 5,322,938, 5,352,605, 5,359,142, and 5,530,196 (35S promoter); U.S. Pat. No. 6,433,252 (maize L3 oleosin promoter); U.S. Pat. No. 6,429,357 (rice actin 2 promoter, and rice actin 2 intron); U.S. Pat. No. 6,294,714 (light-inducible 25 promoters); U.S. Pat. No. 6,140,078 (salt-inducible promoters); U.S. Pat. No. 6,252,138 (pathogen-inducible promoters); U.S. Pat. No. 6,175,060 (phosphorous deficiency-inducible promoters); U.S. Pat. No. 6,388,170 (bidirectional promoters); U.S. Pat. No. 6,635,806 (gamma-coixin promoter); and U.S. Ser. No. 09/757,089 (maize chloroplast aldolase promoter). Additional promoters include the nopaline synthase (NOS) promoter (Ebert et al., 1987) and the octopine synthase (OCS) promoter (both of which are carried on tumor-inducing plasmids of Agrobacterium tumefaciens); the caulimovirus promoters such as the cauliflower mosaic virus (CaMV) 19S promoter (Lawton et al., 1987); the CaMV 35S promoter (Odell et al., 1985); the figwort mosaic virus 35S-promoter (Walker et al., 1987); the sucrose synthase promoter (Yang and Russell, 1990); the R gene complex promoter (Chandler et al., 1989); the chlorophyll a/b binding protein gene promoter; CaMV35S (U.S. Pat. Nos. 5,322,938, 5,352,605, 5,359,142, and 5,530,196); FMV35S (U.S. Pat. Nos. 6,051,753, and 5,378,619); a PC1SV promoter (U.S. Pat. No. 5,850,019); the SCP1 promoter (U.S. Pat. No. 6,677,503); and AGRtu.nos promoters (GenBank Accession No. V00087; Depicker et al., 1982; Bevan et al., 1983).

For expression in vegetative tissue leaf-specific promoters, such as the ribulose biphosphate carboxylase (RBCS) promoters, can be used. For example, the tomato RBCS1, RBCS2 and RBCS3A genes are expressed in leaves and light grown seedlings (Meier et al., 1997). A ribulose bisphosphate carboxylase promoters expressed almost exclusively in mesophyll cells in leaf blades and leaf sheaths at high levels, described by Matsuoka et al. (1994), can be used. Another leaf-specific promoter is the light harvesting chlorophyll a/b binding protein gene promoter (see, Shiina et al., 1997). The Arabidopsis thaliana myb-related gene promoter (Atmyb5) described by Li et al. (1996), is leaf-specific. The Atmyb5 promoter is expressed in developing leaf trichomes, stipules, and epidermal cells on the margins of young rosette and cauline leaves, and in immature seeds. A leaf promoter identified in maize by Busk et al. (1997), can also be used. Additional promoters for modulating endogenous pathways are described in Kohler et al., 1996, Noh and Amasino 1999, Borghi (2010) and Corrado and Karali (2009).

Additional regulatory sequences that may optionally be operably linked to a polynucleotide may include 5′ UTRs located between a promoter sequence and a coding sequence that function as a translation leader sequence. The translation leader sequence is present in the fully-processed mRNA, and it may affect processing of the primary transcript, and/or RNA stability. Examples of translation leader sequences include maize and petunia heat shock protein leaders (U.S. Pat. No. 5,362,865), plant virus coat protein leaders, plant rubisco leaders, and others (see, e.g., Turner and Foster, 1995). Nonlimiting examples of 5′UTRs include GmHsp (U.S. Pat. No. 5,659,122); PhDnaK (U.S. Pat. No. 5,362,865); AtAntl; TEV (Carrington and Freed, 1990); and AGRtunos (GenBank Accession No. V00087; and Bevan et al., 1983).

In some embodiments, the promoter region is derived from chloroplast genes, such as the psbA gene from spinach or pea, the rbcL and atpB promoter region from maize and rRNA promoters (from the plastid rrn operon). Examples of promoters are described in Verma Daniell (2007), Rasala et al. (2011), Hanley-Bowdoin and Chua, (1987), Mullet et al. (1985); Hanley-Bowdoin (1986) PhD. Dissertation, The Rockefeller University, Krebbers et al. (1982); Zurawski et al. (1981) and Zurawski et al. (1982). Other promoters may be identified and the relative strength of promoters so identified evaluated, by placing a promoter of interest 5′ to a promoterless marker gene and observing its effectiveness relative to transcription obtained from, for example, the promoter from the psbA gene, the strongest chloroplast promoter identified to date. The efficiency of coding region expression additionally may be enhanced by a variety of techniques. These include the use of multiple promoters inserted in tandem 5′ to the DNA sequence of interest, for example a double psbA promoter, the addition of enhancer sequences and the like.

In a preferred embodiment, the nucleic acid construct is any one of those listed in the Examples and may optionally be comprised within a vector. In another embodiment, the nucleic acid construct may include a long intergenic region (LIR), promoter or terminator described in the Examples or known in the art.

In an aspect of the invention, there is provided methods for the integration of the polynucleotides or nucleic acid constructs of the invention into either the nuclear genomic DNA or plastid (such as chloroplast) genomic DNA of a plant or alga cell. In the former case, it is envisaged that the nucleic acid constructs will contain polynucleotides that encode a fusion polypepide comprising a plastid transporting peptide. In the latter case, the nucleic acid constructs will not necessarily contain polynucleotides that encode a fusion polypepide comprising a plastid transporting peptide.

Where chloroplast genomic DNA integration is contemplated, one or more integration sites selective for integration of the construct into a chloroplast genome of a plant or alga is required. Examples of chloroplast integration regions include but are not limited to tmV-3′-rpsl2, tmI-tmA, and trnfM-trnG. Examples of specific chloroplast integration sites include but are not limited to tmH/pbA, trnG/trnfM, ycf3/tmS, rbcL/accD, petA/psbJ, 5′rps12/clpP, petD/rpoA, ndhB/rps7, 3′rpsl2/trnV, tmV/rml6, rml6/tml, tml/tmA, tmN/tmR, and rp32/tmL.

### Vectors

In another embodiment of the invention, polynucleotides described herein may be contained within a vector, preferably one that is suitable for expression in a plant, alga or a plastid thereof. In another embodiment, a host cell is transformed with a vector or nucleic acid molecule comprising a nucleic acid sequence as described herein. In another embodiment, the host cell is any microorganism as described herein. As used herein, the term “vector” or “nucleic acid vector” means a nucleic acid molecule, such as a plasmid, comprising regulatory elements and a site for introducing DNA, which is used to introduce said DNA into a plant, alga, cell thereof or plastid. The DNA can encode a heterologous protein, which can be expressed in a plant, alga, cell thereof or plastid described herein and used to increase production of a cannabinoid described herein. The transgenic DNA can be integrated into nuclear, mitochondrial or chloroplastic genomes through homologous or nonhomologous recombination. The DNA can also replicate without integrating into nuclear, mitochondrial or chloroplastic genomes in an extra-chromosomal vector. The vector can contain a single, operably-linked set of regulatory elements that includes a promoter, a 5′ untranslated region (5′ UTR), an insertion site for transgenic DNA, a 3′ untranslated region (3′ UTR) and a terminator sequence. Vectors useful in the present methods are well known in the art.

The vector can be either RNA or DNA, either prokaryotic or eukaryotic, and typically is a viral vector, derived from a virus, or a plasmid. Plasmid vectors typically include additional nucleic acid sequences that provide for easy selection, amplification, and transformation of the expression cassette in prokaryotic cells, e.g., pUC-derived vectors, pSK-derived vectors, pGEM-derived vectors, pSP-derived vectors, pBS-derived vectors, or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

In another embodiment, the vector is a commercially-available vector. As used herein, the term “expression cassette” means a single, operably-linked set of regulatory elements that includes a promoter, a 5′ untranslated region (5′ UTR), an insertion site for transgenic DNA, a 3′ untranslated region (3′ UTR) and a terminator sequence. In an embodiment, the at least one polynucleotide is an episomal vector. In a preferred embodiment, the vector is any one of those listed in Table 3. In a more preferred embodiment, the vector includes elements of a viral vector to enhance expression (Diamos and Mason 2019) for instance, by utilising the bean yellow dwarf viral (BeYDV) vector replication machinery as described in Example 5 herein. In this respect, in a preferred embodiment, elements of the BeYDV including the replication machinery are capable of providing strong expression of the polynucleotides described herein. In particular, in an embodiment, two replication proteins, Rep and RepA, produced on the complementary sense DNA strand (C1/C2 genes) may be utilised. Rep and RepA are produced from a single intron-containing transcript: RepA is the predominant protein product from the unspliced transcript, while a relatively uncommon excision of an intron alters the reading frame to produce Rep. In this example, production of the viral proteins is driven by a single bidirectional promoter in the long intergenic region (LIR) which also contains the viral origin of replication. Both divergent transcripts converge at a short intergenic region (SIR), which has bidirectional transcription terminator signals and is suspected to be the origin of complementary strand synthesis (Liu et al., 1998).

In addition, polynucleotide, nucleic acid construct or vector may contain one or more introns in front of the cloning site or within a gene sequence to drive a strong expression of the gene of interest. The one or more introns may include introns of FBAC2-1 TUFA-1, EIF6-1, RPS4-1, RbcS2-1, RbcS2-2. The polynucleotide, nucleic acid construct or vector may contain more than one intron or more than one copy of the same intron. The polynucleotide, nucleic acid construct or vector may also contain a suitable terminator such as tEF-1 a, t40SRPS8, tH4-1 B, ty-Tubulin, tRBCMT, tFcpB, tFcpC, tFcpD, PAL, tFcpA, tRbcS2. Seletctable marker genes can also be linked on the vector, such as the kanamycin resistance gene (also known as neomycin phosphotransferase gene II, or nptII), zeocin resistance gene, hygromycin resistance gene, Basta resistance gene, hygromycin resistance gene, or others.

In accordance with the invention, each expression vector may contain a promoter that drives transcription in a plant, alga or plant cell. As used herein, an “expression vector” is a DNA or RNA vector that is capable of transforming a host cell and of effecting expression of one or more specified polynucleotides. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in algal and plant cells.

A polynucleotide, recombinant nucleic acid construct or vector of the present invention may also include a screenable marker. Screenable markers may be used to monitor expression. Exemplary screenable markers include a p-glucuronidase or uidA gene (GUS) which encodes an enzyme for which various chromogenic substrates are known (Jefferson et al., 1987); an R-locus gene, which encodes a product that regulates the production of anthocyanin pigments (red color) in plant tissues (Dellaporta et al., 1988); a β-lactamase gene (Sutcliffe et al., 1978); a gene which encodes an enzyme for 20 which various chromogenic substrates are known (e.g., PADAC, a chromogenic cephalosporin); a luciferase gene (Ow et al., 1986); a xylE gene that encodes a catechol dioxygenase that can convert chromogenic catechols (Zukowski et al., 1983); an amylase gene (Ikatu et al., 1990); a tyrosinase gene which encodes an enzyme capable of oxidizing tyrosine to DOPA and dopaquinone which in turn condenses to melanin (Katz et al., 1983); and an alpha-galactosidase.

As used herein, the term “tag” refers to an amino acid sequence that is recognized by an antibody. The tag amino acid sequence links to, for example, sequence of an enzyme, thereby allowing detection or isolation of the enzyme by the binding between the tag and the tag-specific antibody. For example, common tags known in the art include 6His, MYC, FLAG, V5, HA, and HSV. These tags are useful when positioned at the N- or C-terminus.

As used herein, the term “reporter” refers to a molecule that allows for the detection of another molecule to which the reporter is attached or associated, or for the detection of an organism that comprises the reporter. Reporters can include fluorescent molecules including fluorescent proteins such as green fluorescent protein (GFP), yellow fluorescent protein (YFP), mNeonGreen and red fluorescent protein (RFP). In some embodiments, the at least one nucleic acid molecule comprises one or more reporter sequences encoding a reporter with an amino acid sequence with at least 70%, at least 80%, at least 90%, at least 95%, at least 96%, at least 97%, at least 98%, at least 99%, or 100% identical to the nucleotide sequence as set forth in SEQ ID NO:16.

Nucleic acid sequences as described herein can be provided in vectors in different arrangements or combinations. Each individual sequence that encodes a component of the cannabinoid biosynthetic pathway can be provided in separate vectors. Alternatively, multiple sequences can be provided together in the same vector. For example, polynucleotides encoding a polyketide synthase and an olivetolc acid cyclase (OAC), an acyl activating enzyme 1 (AAE1) and an olivetol synthase (OLS) can be provided together in a first vector, and a polynucleotide encoding a prenyltransferase can be provided in a second vector. Alternatively, these sequences can be provided together in the same vector. Where more than one polynucleotide is provided in the same vector, the sequences can be provided in separate expression cassettes, or together in the same expression cassette.

### Enhancing RNA Expression Levels and Stabilised Expression

Post-transcriptional gene silencing (PTGS) is a nucleotide sequence-specific defense mechanism that can target both cellular and viral mRNAs for degradation. PTGS occurs in plants stably or transiently transformed with a recombinant polynucleotide(s) and results in the reduced accumulation of RNA molecules with sequence similarity to the introduced polynucleotide. “Post-transcriptional” is understood to refer to a mechanism operating at least partly, but not necessarily exclusively, after production of an initial RNA transcript, for example during processing of the initial RNA transcript, or concomitant with splicing or export of the RNA to the cytoplasm, or within the cytoplasm by complexes associated with Argonaute proteins.

RNA molecule levels can be increased, and/or RNA molecule levels stabilized over numerous generations by limiting the expression of a silencing suppressor in a plant or part thereof. As used herein, a “silencing suppressor” is any polynucleotide or polypeptide that can be expressed in a plant cell that enhances the level of expression product from a different introduced gene in the plant cell, particularly, over repeated generations from the initially genetically engineered plant. In an embodiment, the silencing suppressor is a viral silencing suppressor or mutant thereof. A large number of viral silencing suppressors are known in the art and include, but are not limited to P19, V2, P38, Pe-Po and RPV-P0. Examples of suitable viral silencing suppressors include those described in WO 2010/057246. A silencing suppressor may be stably expressed in a plant or part thereof of the present invention.

As used herein, the term “stably expressed” or variations thereof refers to the level of the RNA molecule being essentially the same or higher in progeny plants over repeated generations, for example, at least three, at least five, or at least ten generations, when compared to corresponding plants lacking the exogenous polynucleotide encoding the silencing suppressor. However, this term(s) does not exclude the possibility that over repeated generations there is some loss of levels of the RNA molecule when compared to a previous generation, for example, not less than a 10% loss per generation.

The suppressor can be selected from any source e.g. plant, viral, mammal, etc. The suppressor may be, for example, flock house virus B2, pothos latent virus P14, pothos latent virus AC2. African cassava mosaic virus AC4, bhendi yellow vein mosaic disease C2, bhendi yellow vein mosaic disease C4, bhendi yellow vein mosaic disease βC1, tomato chlorosis virus p22, tomato chlorosis virus CP, tomato chlorosis virus CPm, tomato golden mosaic virus AL2, tomato leaf curl Java virus βC1, tomato yellow leaf curl virus V2, tomato yellow leaf curl virus-China C2, tomato yellow leaf curl China virus Y10 isolate βC1, tomato yellow leaf curl Israeli isolate V2, mungbean yellow mosaic virus-Vigna AC2, hibiscus chlorotic ringspot virus CP, turnip crinkle virus P38, turnip crinkle virus CP, cauliflower mosaic virus P6, beet yellows virus p21, citrus tristeza virus p20, citrus tristeza virus p23, citrus tristeza virus CP, cowpea mosaic virus SCP, sweet potato chlorotic stunt virus p22, cucumber mosaic virus 2b, tomato aspermy virus HC-Pro, beet curly top virus L2, soil borne wheat mosaic virus 19K, barley stripe mosaic virus Gammab, poa semilatent virus Gammab, peanut clump pecluvirus P15, rice dwarf virus Pns10, curubit aphid borne yellows virus P0, beet western yellows virus P0, potato virus X P25, cucumber vein yellowing virus P1b, plum pox virus HC-Pro, sugarcane mosaic virus HC-Pro, potato virus Y strain HC-Pro, tobacco etch virus P1/HC-Pro, turnip mosaic virus P1/HC-Pro, cocksfoot mottle virus P1, cocksfoot mottle virus-Norwegian isolate P1, rice yellow mottle virus P1, rice yellow mottle virus-Nigerian isolate P1, rice hoja blanca virus NS3, rice stripe virus NS3, crucifer infecting tobacco mosaic virus 126K, crucifer infecting tobacco mosaic virus p122, tobacco mosaic virus p122, tobacco mosaic virus 126, tobacco mosaic virus 130K, tobacco rattle virus 16K, tomato bushy stunt virus P19, tomato spotted wilt virus NSs, apple chlorotic leaf spot virus P50, grapevine virus A p10, grapevine leafroll associated virus-2 homolog of BYV p21, as well as variants/mutants thereof. The list above provides the virus from which the suppressor can be obtained and the protein (e.g., B2, P14, etc.), or coding region designation for the suppressor from each particular virus. Other candidate silencing suppressors may be obtained by examining viral genome sequences for polypeptides encoded at the same position within the viral genome, relative to the structure of a related viral genome comprising a known silencing suppressor, as is appreciated by a person of skill in the art.

Silencing suppressors can be categorized based on their mode of action. Suppressors such as V2 which preferentially bind to a double-stranded RNA molecule which has overhanging 5′ ends relative to a corresponding double-stranded RNA molecule having blunt ends are particularly useful for enhancing transgene expression when used in combination with gene silencing (exogenous polynucleotide encoding a dsRNA). Other suppressors such as p19 which preferentially bind a dsRNA molecule which is 21 base pairs in length relative to a dsRNA molecule of a different length can also allow transgene expression in the presence of an exogenous polynucleotide encoding a dsRNA, but generally to a lesser degree than, for example, V2. This allows the selection of an optimal combination of dsRNA, silencing suppressor and over-expressed transgene for a particular purpose. Such optimal combinations can be identified using a method of the invention. Multiple copies of a suppressor may be used. Different suppressors may be used together (e.g., in tandem).

In an embodiment, the silencing suppressor preferentially binds to a double-stranded RNA molecule which has overhanging 5′ ends relative to a corresponding double-stranded RNA molecule having blunt ends. In this context, the corresponding double-stranded RNA molecule preferably has the same nucleotide sequence as the molecule with the 5′ overhanging ends, but without the overhanging 5′ ends. Binding assays are routinely performed, for example in in vitro assays, by any method as known to a person of skill in the art.

In a particular example, the plants produced increased levels of enzymes for cannabinoid production in plants such as Nicotania or Brassicas.

### Codon Optimisation

It will be understood that it is possible to improve the expression of a heterologous nucleic acid in a host organism or host cell by replacing the nucleotide sequences coding for a particular amino acid (i.e., a codon) with another codon which is better expressed in the host organism (i.e., codon optimization). One reason that this effect arises is due to the fact that different organisms show preferences for different codons. In some embodiments, a heterologous polynucleotide disclosed herein is modified or optimized such that the nucleotide sequence reflects the codon preference for the particular host cell, preferably a plant or alga cell.

For example, the polynucleotide may in some embodiments be modified or optimized for plant codon preference, preferably of a vascular plant, more preferably of the genus Nicotiana, preferably Nicotiana benthamiana or Nicotiana tabacum. Alternatively, the polynucleotide may in some embodiments be modified or optimized for alga codon preference using methods known in the art, for specific algae useful in the present invention including Chlamydomonas sp. Dunaliella sp, Chlorella sp. In another embodiment, the polynucleotide may be further modified or optimized for plastid expression in plants, parts thereof or algae.

Statistical methods have been generated to analyze codon usage bias in various plants or algae and many computer algorithms have been developed to implement these statistical analyses in the design of codon optimized gene sequences. Other modifications in codon usage to increase protein expression that are not dependent on codon bias have also been described.

It will be appreciated that this disclosure embraces the degeneracy of codon usage as would be understood by one of ordinary skill in the art and illustrated in Table 2.

TABLE 2
Exemplary substitutions.

| Original Residue | Exemplary Substitutions |
|---|---|
| Ala (A) | GCT, GCC, GCA, GCG |
| Arg (R) | CGT, CGC, CGA, CGG, AGA AGG |
| Asn (N) | AAT, AAC |
| Asp (D) | GAT, GAC |
| Cys (C) | TGT, TGC |
| Gln (Q) | CAA, CAG |
| Glu (E) | GAA, GAC |
| Gly (G) | GGT, GGC, GGA, GGG |
| His (H) | CAT, CAC |
| Ile (I) | ATT, ATC, ATA |
| Leu (L) | TTA, TTG, CTT, CTC, CTA, CTG |
| Lys (K) | AAA, AAG |
| Met (M) | ATG |
| Phe (F) | TTT, TTC |
| Pro (P) | CCT, CCC, CCA, CCG |
| Ser (S) | TCT, TCC, TCA, TCG, AGT, AGC |
| Thr (T) | ACT, ACC, ACA, ACG |
| Trp (W) | TGG |
| Tyr (Y) | TAT, TAC |
| Val (V) | GTT, GTC, GTA, GTG |

### Recombinant Cells

Another embodiment of the present invention includes a recombinant cell comprising a host cell, preferably a plant or alga cell transformed with one or more exogenous polynucleotides, nucleic acid constructs or vectors of the present invention, or progeny cells thereof. Transformation of polynucleotides, nucleic acid constructs or vectors into a cell can be accomplished by any method by which a nucleic acid molecule can be inserted into the cell. Transformation techniques include, but are not limited to, transfection, particle bombardment/biolistics, electroporation, microinjection, lipofection, adsorption, and protoplast fusion. In an embodiment, gene editing may be used to transform the target cell using, for example, targeting nucleases such as TALEN, MADS7, Cpf1 or Cas9-CRISPR or engineered nucleases derived therefrom.

A recombinant cell may remain unicellular or may grow into a tissue, organ or a multicellular organism. Transformed nucleic acid constructs of the present invention can remain extrachromosomal or can integrate into one or more sites within a chromosome of the transformed (i.e., recombinant) cell in such a manner that their ability to be expressed is retained. Preferred host cells are plant or alga cells, more preferably cells of a plant.

Host cells into which the polynucleotide(s) are introduced can be either untransformed cells or cells that are already transformed with at least one nucleic acid. Such nucleic acids may be related to lipid synthesis, cannabinoid synthesis, or unrelated. Host cells of the present invention either can be endogenously (i.e., naturally) capable of producing polypeptide(s) defined herein, in which case the recombinant cell derived therefrom has an enhanced capability of producing the polypeptide(s), or can be capable of producing said polypeptide(s) only after being transformed with at least one polynucleotide of the invention. In an embodiment, a recombinant cell of the invention has an enhanced capacity to produce cannabinoids.

### Nuclease Genetic Engineering

Endonucleases can be used to generate single strand or double strand breaks in genomic DNA. The genomic DNA breaks in eukaryotic cells are repaired using non-homologous end joining (NHEJ) or homology directed repair (HDR) pathways. NHEJ may result in imperfect repair resulting in unwanted mutations and HDR can enable precise gene insertion by using an exogenous supplied repair DNA template. CRISPR-associated (Cas) proteins have received significant interest although transcription activator-like effector nucleases (TALENs) and zinc-finger nucleases are still useful, the CRISPR-Cas system offers a simpler, versatile and cheaper tool for genome modification (Doudna and Charpentier, 2014).

The CRISPR-Cas systems are classed into three major groups using various nucleases or combinations on nuclease. In class 1 CRISPR-Cas systems (types I, III and IV), the effector module consists of a multi-protein complex whereas class 2 systems (types II, V and VI) use only one effector protein (Makarova et al., 2015). Cas includes a gene that is coupled or close to or localised near the flanking CRISPR loci. Haft et al. (2005) provides a review of the Cas protein family.

The nuclease is guided by the synthetic small guide RNA (sgRNAs or gRNAs) that may or may not include the tracRNA resulting in a simplification of the CRISPR-Cas system to two genes; the endonuclease and the sgRNA (Jinek et al. 2012). The sgRNA is typically under the regulatory control of a U3 or U6 small nuclear RNA promoter. The sgRNA recognises the specific gene and part of the gene for targeting. The protospacer adjacent motif (PAM) is adjacent to the target site constraining the number of potential CRISPR-Cas targets in a genome although the expansion of nucleases also increases the number of PAM's available. There are numerous web tools available for designing gRNAs including CHOPCHOP (http://chopchop.cbu.uib.no), CRISPR design https://omictools.com/crispr-design-tool, E-CRISP http://www.e-crisp.org/E-CRISP/, Geneious or Benchling https://benchling.com/crispr.

CRISPR-Cas systems are the most frequently adopted in eukaryotic work to date using a Cas9 effector protein typically using the RNA-guided Streptococcus pyogenes Cas9 or an optimised sequence variant in multiple plant species (Luo et al., 2016). Luo et al. (2016) summarises numerous studies where genes have been successfully targeted in various plant species to give rise to indels and loss of function mutant phenotypes in the endogenous gene open reading frame and/or promoter. Due to the cell wall on plant cells the delivery of the CRISPR-Cas machinery into the cell and successful transgenic regenerations have used Agrobacterium tumefaciens infection (Luo et al., 2016) or plasmid DNA particle bombardment or biolistic delivery. Vectors suitable for cereal transformation include pCXUNcas9 (Sun et al, 2016) or pYLCRISPR/Cas9Pubi-H available from Addgene (Ma et al., 2015, accession number KR029109.1).

Alternative CRISPR-Cas systems refer to effector enzymes that contain the nuclease RuvC domain but do not contain the HNH domain including Cas12 enzymes including Cas12a, Cas12b, Cas12f, Cpf1, C2c1, C2c3, and engineered derivatives. Cpf1 creates double-stranded breaks in a staggered manner at the PAM-distal position and being a smaller endonuclease may provide advantages for certain species (Begemann et al., 2017). Other CRISPR-Cas systems include RNA-guided RNAses including Cas13, Cas13a (C2c2), Cas13b, Cas13c.

### Sequence Insertion or Integration

The CRISPR-Cas system can be combined with the provision of a polynucleotide to direct homologous repair for the insertion of a sequence into a genome. Targeted genome integration of plant transgenes enables the sequential addition of transgenes at the same locus. This “cis gene stacking” would greatly simplify subsequent breeding efforts with all transgenes inherited as a single locus. When coupled with CRISPR/Cas9 cleavage of the target site the transgene can be incorporated into this locus by homology-directed repair that is facilitated by flanking sequence homology. This approach can be used to rapidly introduce new alleles without linkage drag or to introduce allelic variants that do not exist naturally.

### Nickases

The CRISPR-Cas II systems use a Cas9 nuclease with two enzymatic cleavage domains a RuvC and HNH domain. Mutations have been shown to alter the double strand cutting to single strand cutting and resulting in a technology variant referred to as a nickase or a nuclease-inactivated Cas9. The RuvC subdomain cleaves the non-complementary DNA strand and the HNH subdomain cleaves that DNA strand complementary to the gRNA. The nickase or nuclease-inactivated Cas9 retains DNA binding ability directed by the gRNA. Mutations in the subdomains are known in the art for example S. pyogenes Cas9 nuclease with a D10A mutation or H840A mutation.

### Genome Base Editing or Modification

Base editors have been created by fusing a deaminase with a Cas9 domain (WO 2018/086623). By fusing the deaminase, one can take advantage of the sequence targeting directed by the gRNA to make targeted cytidine (C) to uracil (U) conversion by deamination of the cytidine in the DNA. The mismatch repair mechanisms of the cell then replace the U with a T. Suitable cytidine deaminases may include APOBEC1 deaminase, activation-induced cytidine deaminase (AID), APOBEC3G and CDA1. Further, the Cas9-deaminase fusion may be a mutated Cas9 with nickase activity to generate a single strand break. It has been suggested that the nickase protein was potentially more efficient in promoting homology-directed repair (Luo et al., 2016).

### Vector Free Genome Editing or Genome Modification

More recently methods to use vector free approaches using Cas9/sgRNA ribonucleoproteins have been described with successful reduction of off-target events. The method requires in vitro expression of Cas9 ribonucleoproteins (RNPs) which are transformed into the cell or protoplast and does not rely on the Cas9 being integrated into the host genome, thereby reducing the undesirable side cuts that has been linked with the random integration of the Cas9 gene. Only short flanking sequences are required to form a stable Cas9 and sgRNA stable ribonucleoprotein in vitro. Woo et al. (2015) produced pre-assembled Cas9/sgRNA protein/RNA complexes and introduced them into protoplasts of Arabidopsis, rice, lettuce and tobacco and targeted mutagenesis frequencies of up to 45% observed in regenerated plants. RNP and in vitro demonstrated in several species including dicot plants (Woo et al., 2015), and monocots maize (Svitashev et al., 2016) and wheat (Liang et al., 2017). Genome editing of plants using CRISPR-Cas 9 in vitro transcripts or ribonucleoproteins are fully described in Liang et al. (2018) and Liang et al. (2019).

### Method for Gene Insertion

Plant embryos may be bombarded with a Cas9 gene and sgRNA gene targeting the site of integration along with the DNA repair template. DNA repair templates are may be synthesised DNA fragment or a 127-mer oligonucleotide, with each encoding the cDNA or the gene of interest. Bombarded cells are grown on tissue culture medium. DNA extracted from callus or TO plants leaf tissue using CTAB DNA extraction method can be analysed by PCR to confirm gene integration. T1 plants selected if pcr confirms presence of the gene of interest.

The method comprises introducing into a plant cell the polynucleotide of interest referred to as the donor DNA and the endonuclease. The endonuclease generates a break in the target site allowing the first and second regions of homology of the donor DNA to undergo homologous recombination with their corresponding genomic regions of homology. The cut genomic DNA acts as an acceptor of the DNA sequence. The resulting exchange of DNA between the donor and the genome results in the integration of the polynucleotide of interest of the donor DNA into the strand break in the target site in the plant genome, thereby altering the original target site and producing an altered genomic sequence.

The donor DNA may be introduced by any means known in the art. For example, a plant having a target site is provided. The donor DNA may be provided to the plant by known transformation methods including, Agrobacterium-mediated transformation or biolistic particle bombardment. The RNA guided Cas or Cpf1 endonuclease cleaves at the target site, the donor DNA is inserted into the transformed plant's genome.

Although homologous recombination occurs at low frequency in plant somatic cells the process appears to be increased/stimulated by the introduction of doublestrand breaks (DSBs) at selected endonuclease target sites. Ongoing efforts to generate Cas, in particular Cas9, variants or alternatives such as Cpf1 or Cms1 may improve the efficiency.

### Genetically Modified Plants or Algae

The present invention provides genetically engineered plants, algae or parts thereof, or plastids thereof, where the plants, parts thereof, algae, or plastids thereof, are genetically engineered with one or more polynucleotides disclosed herein to increase production of a cannabinoid.

In an embodiment, the genetically engineered plant is a plant of a genus other than Cannabis or is a non-Cannabis plant. In another embodiment, the genetically engineered plant part is an aerial plant part, preferably of a vascular plant, more preferably leaves or stems of a vascular plant. A non-limiting example is the leaves of Nicotiana sp., preferably Nicotiana benthamiana or Nicotiana tabacum. Where plant parts are contemplated for use in the invention, a suitable plant part may include roots, stems, leaves, flowers, fruits, and seeds, preferably the leaves and seeds. Where the term “aerial” plant part is used, this will be understood to mean parts of the plant that are above the ground including stems, leaves, flowers, fruits and seeds.

In an embodiment, the genetically engineered plant is a lower plant (non-vascular) or a higher plant (vascular), preferably a vascular plant. A vascular plant will be understood to be those plants that contain vascular tissues such as xylem (for transporting water) and phloem (for transporting minerals and nutrients) and are also known as tracheophytes. Vascular plants are also homoiohydric (capable of regulating water concentration), possess true leaves, roots, and stems and include ferns, clubmosses, horsetails, seed plants, angiosperms, and gymnosperms. In contrast, non-vascular plants or lower plants lack vascular tissues, are poikilohydric (lack mechanism against dessication), lack true leaves, roots, and stems, include mosses, liverworts and hornworts and generally inhabit damp, swampy places. A skilled person will understand that this distinction is well characterised in the art. It will be understood that plants known to belong to the non-vascular and vascular classifications may be suitable to use in accordance with the invention.

In an embodiment, the plastid is not from an alga. Where algae are however contemplated for use in the invention, it will be understood that algae defines a large and diverse group of photosynthetic eukaryotic organisms. Suitable algae for use in the invention may include, but are not limited to Euglenophyta (Euglenoids); Chrysophyta (Golden-brown algae and Diatoms); Pyrrophyta (Fire algae); Chlorophyta (Green algae); Rhodophyta (Red algae); Paeophyta (Brown algae) and Xanthophyta (Yellow-green algae). Specific non-limiting examples algae include the Classes: Chlorophyceae, Eustigmatophyccae, Prymnesiophyceae, Bacillariophyceae, Bacillariophytes capable of oil production include the genera Amphipleura, Amphora, Chaetoceros, Cyclotella, Cymbella, Fragilaria, Hantzschia, Navicula, Nitzschia, Phaeodactylum, and Thalassiosira. Specific non-limiting examples of chlorophytes capable of oil production include Ankistrodesmus, Botryococcus, Chlorella, Chlorococcum, Dunaliella, Monoraphidium, Oocystis, Scenedesmus, and Tetraselmis. In one aspect, the chlorophytes can be Chlorella or Dunaliella. Specific non-limiting examples of cyanophytes capable of oil production include Oscillatoria and Synechococcus. A specific example of chrysophytes capable of oil production includes Boekelovia. Specific non-limiting examples of haptophytes include Isochysis and Pleurochysis.

Specific algae useful in the present invention include, for example, Chlamydomonas sp. such as Chlamydomonas reinhardtii, Dunaliella sp. such as Dunaliella salina, Dunaliella tertiolecta, D. acidophila, D. bardawil, D. bioculata, D. lateralis, D. maritima, D. minuta, D. parva, D. peircei, D. polymorpha, D. primolecta, D. pseudosalina, D. quartolecta, D. viridis, Haematococcus sp., Chlorella sp. such as Chlorella vulgaris, Chlorella sorokiniana or Chlorella protothecoides, Thraustochytrium sp., Schizochytrium sp., Volvox sp. Nannochloropsis sp., Botryococcus braunii which can contain over 60 wt % lipid, Phaeodactylum tricornutum, Thalassiosira pseudonana, Isochrysis sp., Pavlova sp., Chlorococcum sp, Ellipsoidion sp., Neochloris sp., Scenedesmus sp.

In a further embodiment, the plant is a high biomass plant. High biomass plants will be understood to be a plants that are capable of production of higher amounts of a cannabinoid or industrial product described herein or known in the art when compared to a lower biomass plant. High biomass plants typically have a higher mass in a given area when compared to plants of lower biomass. It will be understood that high biomass plants are preferable for use in the invention. Such high biomass plants may include but are not limited to Acrocomia aculeata (macauba palm), Arabidopsis thaliana, Aracinis hypogaea (peanut), Astrocaryum murumuru (murumuru), Astrocaryum vulgare (tucuma), Attalea geraensis (Indaia-rateiro), Attalea humilis (American oil palm), Attalea oleifera (andaia), Attalea phalerata (uricuri), Attalea speciosa (babassu), Avena sativa (oats), Beta vulgaris (sugar beet), Brassica sp. such as Brassica carinata, Brassica juncea, Brassica napobrassica, Brassica napus (canola), Camelina sativa (false flax), Cannabis sativa (hemp), Carthamus tinctorius (safflower), Caryocar brasiliense (pequi), Cocos nucifera (Coconut), Crambe abyssinica (Abyssinian kale), Cucumis melo (melon), Elaeis guineensis (African palm), Glycine max (soybean), Gossypium hirsutum (cotton), Helianthus sp. such as Helianthus annuus (sunflower), Hordeum vulgare (barley), Jatropha curcas (physic nut), Joannesia princeps (arara nut-tree), Lemna sp. (duckweed) such as Lemna aequinoctialis, Lemna disperma, Lemna ecuadoriensis, Lemna gibba (swollen duckweed), Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Licania rigida (oiticica), Linum usitatissimum (flax), Lupinus angustifolius (lupin), Mauritia flexuosa (buriti palm), Maximiliana maripa (inaja palm), Miscanthus sp. such as Miscanthus x giganteus and Miscanthus sinensis, Nicotiana sp. (tobacco) such as Nicotiana tabacum or Nicotiana benthamiana, Oenocarpus bacaba (bacaba-do-azeite), Oenocarpus bataua (pataua), Oenocarpus distichus (bacaba-de-leque), Oryza sp. (rice) such as Oryza sativa and Oryza glaberrima, Panicum virgatum (switchgrass), Paraqueiba paraensis (mari), Persea amencana (avocado), Pongamia pinnata (Indian beech), Populus trichocarpa, Ricinus communis (castor), Saccharum sp. (sugarcane), Sesamum indicum (sesame), Solanum tuberosum (potato), Sorghum sp. such as Sorghum bicolor, Sorghum vulgare, Theobroma grandiforum (cupuassu), Trifolium sp., Trithrinax brasiliensis (Brazilian needle palm), Triticum sp. (wheat) such as Triticum aestivum and Zea mays (corn).

In a preferred embodiment, the leaves of a high biomass plant listed above are utilised for the production of the cannabinoid in accordance with the invention. In another preferred embodiment, the chloroplasts of the leaves of a high biomass plant listed above are utilised for the production of the cannabinoid in accordance with the invention.

As used herein, the term “genetically engineered”, “genetically modified” and their derivatives refer to a plant, part thereof, alga or plastid thereof whose genetic material has been altered using molecular biology techniques such as but not limited to molecular cloning, recombinant DNA methods, gene editing, transformation and gene transfer. The genetically engineered plant, part thereof, alga, or plastid thereof includes a living modified plant, part thereof, alga or plastid thereof, genetically engineered plant, part thereof, alga or plastid thereof or a transgenic plant, part thereof, alga or plastid thereof. Genetic alteration includes addition, deletion, modification and/or mutation of genetic material. Such genetic engineering as described herein in the present disclosure increases production of cannabinoids relative to the corresponding wild-type plant, part thereof or alga which has not been genetically engineered for introduction of a polynucleotide of the cannabinoid pathway as described herein. The term wild-type will be understood to mean the same plant or alga that has not been genetically engineered and generally represents the phenotype of the typical form of a given plant or alga as it occurs in nature.

The present disclosure provides a method of producing a cannabinoid in a plant or alga, the method comprising cultivating a plant or alga comprising a nucleic acid construct of the invention under conditions sufficient for expression of the polypeptide in the plant or alga, wherein when expressed in the presence of hexanoic acid (C6) and geranyl-pyrophosphate (GPP), the polypeptides increase the production of cannabigerolic acid (CBGA) in the plant or alga when compared to a wild type plant or alga.

In an embodiment, the nuclear and/or plastid genome of the genetically engineered plant or alga comprises a polynucleotide. In some embodiments, the transgenic plant or alga is homozygous for the genetic modification. In some embodiments, the transgenic plant or alga is heterozygous for the genetic modification.

In an embodiment, production of CBGA is increased by at least about 1.5-about 2 fold, about 2-about 2.5 fold, about 2.5-about 3.0 fold, about 3-about 3.5 fold, about 3.5-about 4.0 fold, about 4-about 4.5 fold, about 4.5-about 5.0 fold, about 5-about 5.5 fold, about 5.5-about 6 fold, about 6-about 6.5 fold, about 6.5-about 7.0 fold, about 7.0-about 7.5 fold, about 7.5-about 8.0 fold, about 8.0-about 8.5 fold, about 8.5-about 9.0 fold, about 9.0-about 9.5 fold or more when compared to a wild-type plant, part thereof, alga or plastid thereof.

In another embodiment, production of CBGA is increased at least about 10%-about 20%, about 20%-about 30%, about 30%-about 40%, about 40%-about 50%, about 50%-about 60%, about 60%-about 70%, about 70%-about 80%, about 80%-about 90%, about 90%-about 100%, about 100%-about 120%, about 120%-about 140%, about 140%-about 160%, about 160%-about 180%, about 180%-about 200%, about 200%-about 220%, about 220%-about 240%, about 240%-about 260%, about 260%-about 280%, about 280%-about 300%, about 300%-about 320%, about 320%-about 340%, about 340%-about 360%, about 360%-about 380%, about 400%-about 420% or more when compared to a wild-type plant, part thereof, alga or plastid thereof.

A skilled person will understand methods for calculating yield of a given cannabinoid. For example, it is known that Cannabis can yield up to 2.8t dry flower/ha at about 15% CBDA which yields about 420 kg of a major cannabinoid/ha. Assuming tobacco biomass in the field is lot dry biomass/ha then ˜4% on a dry weight basis provides for a similar production level. A skilled person will also understand that Cannabis usually accumulates 2-5% CBGA.

In some embodiments, a genetically engineered plant is a transgenic version of a control, non-transgenic plant that normally produces a cannabinoid. In another embodiment, a genetically about plant or alga is a transgenic version of a control, non-transgenic plant or alga that does not normally produce a cannabinoid. In this case, a skilled person will understand the necessary components of a biosynthetic cannabinoid pathway that are required to be expressed in the plant or alga for increased production of a cannabinoid, preferably CBGA when compared to a wild-type plant or alga.

Methods of introducing exogenous polynucleotides into plant and alga cells are well known in the art. Such plant or algae cell are considered “transformed.” Suitable methods may include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, CRISPR/Cas9-mediated genome editing, and the like. The choice of method is generally dependent on the type of cell being transformed and the circumstances under which the transformation is taking place (e.g., in vitro, ex vivo, or in vivo).

A CRISPR/Cas9 system can be used to generate a transgenic (genetically engineered) plant of the present disclosure. CRISPR/Cas9 systems and methods are known in the art (see, e.g., Bortesi and Fischer, 2015; Fan et al., 2015).

Transformation methods based upon the soil bacterium Agrobacterium tumefaciens and man made variants/mutants thereof are probably the best characterized tools used for introducing an exogenous nucleic acid into a vascular plant. The wild type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleotide sequence of interest to be introduced into the plant host. In this respect, a transfer nucleic acid is flanked, typically, by two “border” sequences, although in some instances a single border at one end can be used and the second end of the transferred nucleic acid is generated randomly in the transfer process. A polynucleotide of interest is typically positioned between the left border-like sequence and the right border-like sequence.

Agrobacterium-mediated transformation generally employs cointegrate vectors or, e.g., binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art (see., e.g., Glick and Thompson, (eds.), Methods in Plant Molecular Biology and Biotechnology, Boca Raton, Fla.: CRC Press (1993). In general, it could be assumed that all bacteria that are able to enter the cytosol of the host cell (like S. flexneri or L. monocytogenes) and lyse within this cellular compartment, should be able to transfer DNA.

Agrobacterium-mediated transformation is useful for producing a variety of transgenic vascular plants (Wang et al., 1995) including the high biomass plants suitable for the invention including but not limited to the Nicotiana sp and Brassica sp.

Microprojectile-mediated transformation also can be used to produce a subject transgenic plant. This method, first described by Klein et al. (1987), relies on microprojectiles such as gold or tungsten that are coated with the desired heterologous nucleic acid by precipitation with calcium chloride, spermidine or polyethylene glycol. The microprojectile particles are accelerated at high speed into an angiosperm tissue using a device such as the BIOLISTIC PD-1000 (Biorad; Hercules Calif.).

A polynucleotide may be introduced into a plant or alga in a manner such that the heterologous nucleic acid is able to enter a plant cell(s), e.g., via an in vivo or ex vivo protocol. By “in vivo,” it may mean that the polynucleotide is administered to a living body of a plant or alga e.g. infiltration. By “ex vivo” it may mean that cells or explants are modified outside of the plant or alga, and then such cells or organs are regenerated to a plant or alga. A number of vectors or constructs suitable for stable transformation of plant or alga cells or for the establishment of transgenic plants or algae have been described, including those described in Weissbach and Weissbach, (1989) Methods for Plant Molecular Biology Academic Press, and Gelvin et al. (1990) Plant Molecular Biology Manual, Kluwer Academic Publishers. Specific examples may include those derived from a Ti plasmid of Agrobacterium tumefaciens, as well as those disclosed by Herrera-Estrella et al. (1983), Bevan (1984), and Klee (1985). Alternatively, non-Ti vectors can be used to transfer the DNA into plants and cells by using free DNA delivery techniques. By using these methods transgenic plants such as wheat, rice (Christou, 1991) and corn (Gordon-Kamm, 1990) can be produced. An immature embryo can also be a good target tissue for monocots for direct DNA delivery techniques by using the particle gun (Weeks et al., 1993; Vasil, 1993; Wan and Lemeaux, 1994) and for Agrobacterium-mediated DNA transfer (Ishida et al., 1996).

Methods disclosed for plastid transformation in higher plants include particle gun delivery of DNA containing a selectable marker and targeting of the DNA to the plastid genome through homologous recombination (U.S. Pat. No. 5,451,513. U.S. Pat. Nos. 5,545,818, 5,877,402, 5,932,479, and WO 99/05265). Moreover, exemplary methods for introduction of DNA into chloroplasts are biolistic bombardment, polyethylene glycol transformation of protoplasts, and microinjection (Danieli et al., 1998; Staub et al., 2000; O'Neill et al., 1993; Knoblauch et al., 1999; U.S. Pat. Nos. 5,545,817; 5,576,198; WO 95/16783; Boynton et al., 1993; Svab et al., 1993, McBride et al., 1994).

Any vector suitable for the methods of biolistic bombardment, polyethylene glycol transformation of protoplasts and microinjection will be suitable as a targeting vector for chloroplast transformation. Any double stranded DNA vector may be used as a transformation vector, especially when the method of introduction does not utilize Agrobacterium. Alternatively, methods for transforming a plant or alga of the invention may include any of those described in herein including in the Examples. Plants which can be genetically engineered may include any of those listed herein or known in the art, preferably a high biomass plant, more preferably Nicotiana sp., such as Nicotiana benthamiana or Nicotiana tabacum.

Also provided by the present disclosure are transformed plant or alga cells, tissues, plants or alga and products that contain the transformed plant or alga cells. A feature of the transformed cells, and tissues and parts thereof is the presence of a polynucleotide integrated into the genome, and production by plant or alga cells of one or more polypeptides that are utilized to generate a cannabinoid. Recombinant plant cells of the present disclosure are useful as populations of recombinant cells, or as a tissue, seed, whole plant, stem, fruit, leaf, root, flower, stem, tuber, grain, animal feed, a field of plants, and the like.

Also provided by the present disclosure is reproductive material of a subject transgenic plant or alga, where reproductive material may include seeds, progeny plants and clonal material, where such material can give rise to a plant or alga that produces a cannabinoid according to methods known in the art or described herein.

Although expression of the polynucleotide sequences described herein is in one embodiment preferred in the plastid of a plant, most preferably in a chloroplast of a plant, a skilled person would understand that other organelles including the oil droplet may be suitable for the generation of a cannabinoid described herein.

A plant or alga containing transgenic plastids (e.g., chloroplasts) may be generated when the host cell used in the transformation process possesses totipotency. Procedures for regeneration of transgenic plants from transformed cells or tissues are, in general, similar, with suitable modifications within the capability of one skilled in the art. Regeneration of dicots such as sugar beets, Freytag et al. (1988); tobacco, Svab et al., (1990), or monocots such as wheat from anthers or embryos (see below) routinely has been successful.

If it is desired to transform plastids in other plant or alga materials, for example anther culture derived plants or embryo derived callus, the plant material is placed in a convenient container, for example a petri dish as described above for isolated cells (see also U.S. Pat. No. 6,680,426; Hanson et al (2012); Golds et al. (1993)).

Once the plastid has been shown to have been transformed, the cells of the plant or alga may be used repeatedly for tissue culture, followed by a growth of callus tissue where desired or regeneration of a plant or alga. Thus, the modified plant or alga cell may be repetitively regenerated by use of cell and tissue culture. In some instances, proper propagation may be maintained from seed. In order to improve the ability to identify transformed cells, one may desire to employ a selectable or screenable marker gene, as previously set forth, with the transformation vector used to generate the transformant. In the case where a selectable marker is used, transformed cells are identified within the potentially transformed cell population by exposing the cells to a selective agent or agents. In the case where a screenable marker is used, cells may be screened for the desired marker gene trait.

Cells that survive the exposure to the selective agent, or cells that have been scored positive in a screening assay, may be cultured in media that supports regeneration of plants or alga. In some embodiments, any suitable plant or alga tissue culture media (e.g., MS and N6 media) may be modified by including further substances, such as growth regulators. Tissue may be maintained on a basic medium with growth regulators until sufficient tissue is available to begin plant regeneration efforts, or following repeated rounds of manual selection, until the morphology of the tissue is suitable for regeneration (e.g., at least 2 weeks), then transferred to media conducive to shoot formation. Cultures are transferred periodically until sufficient shoot formation has occurred. Once shoots are formed, they are transferred to media conducive to root formation. Once sufficient roots are formed, plants or alga can be transferred to soil for further growth and maturation.

To confirm the presence of a polynucleotide in the regenerating plants, a variety of assays may be performed. Such assays include, for example: molecular biology assays, such as Southern and Northern blotting, PCR, and nucleic acid sequencing; plant part assays, such as leaf or root assays.

Depending on the transformation approach, the transformants may include cells that are both plastid and nuclear transformants so that selection for a marker linked to plastid transformation might also result in the selection of cells that are both plastid and nuclear transformants. This problem can be addressed by utilising a preferred nucleic acid construct of the invention described above that contains homologous recombination regions (also described as integration sites) that enable integration in the plastid genome, but not the nuclear genome, and use of a promoter that is active in the chloroplasts only. This enables the selection method to select cells or plants that produce the polypeptides in plastids and that do not produce polypeptides outside of plastids.

Integration events may be analyzed, for example, by PCR amplification using, e.g., oligonucleotide primers specific for a polynucleotide of interest. PCR genotyping is understood to include, but not be limited to, polymerase-chain reaction (PCR) amplification of genomic DNA derived from isolated host plant callus tissue predicted to contain a polynucleotide of interest integrated into the genome, followed by standard cloning and sequence analysis of PCR amplification products. Methods of PCR genotyping have been well described (for example, Rios et al., 2002) and may be applied to genomic DNA derived from any plant species (e.g., Z. mays or G. max) or tissue type, including cell cultures.

Once a genetically engineered plant, alga, part thereof or plastid expressing one or more polynucleotides of the invention has been produced, methods known in the art or described herein may be utilised to extract a cannabinoid, preferably CBGA.

In a non-limiting example, extraction from fresh or dry plant or alga parts thereof may involve a combination of steps including physical processing of material, (e.g. ultrasonic-assisted, microwave-assisted, sieve), solvent (e.g., Ethanol, water, isopropyl) extraction, hydrocarbon or CO₂ extraction (e.g. supercritical CO₂ extraction), winterization & filtering, drying, distillation and/or chromatography, depending on the purity required. It will be appreciated by those skilled in the art that the the desired characteristics of the product will influence the method of extraction. For example, cannabinoids can be extracted in acidic form by undertaking processing steps at or below ambient temperature, or cannabinoids can be decarboxylated for extraction into the neutral form by subjecting the material to higher temperatures.

In particular, harvested plant material is first vacuum dried and ground into a powder. The cannabinoid, preferably CBGA is extracted using supercritical gas (typically CO₂) or an organic solvent (e.g. ethanol), thereby removing lighter terpene fraction. The resulting crude cannabinoid extract is then subjected to winterization using cold ethanol to allow for the elimination of waxes and lipids by precipitation. Following filtration and the removal of the alcohol by evaporation, the crude cannabinoid fraction (‘resin’) is concentrated by distillation to eliminate heavy terpenes and other residues. Where extraction of specific cannabinoid molecules is desired, the cannabinoid distillate is subjected to a further chromatography step such as high-performance liquid chromatography, centrifugal partition chromatography, or supercritical fluid chromatography.

One or more known processes known in the art or described herein may then be utilised to convert the extracted cannabinoid to any one of CBG, CBDA, CBD, CBCA, CBC, THCA, THC or another cannabinoid known in the art. In general, this process may include enzymatic conversion, either in vivo (e.g., by yeast feeding as disclosed in Zirpel et al. (2015); by leaf infiltration e.g., Geissler et al. (2018), and Gulck et al. (2020) or in vitro (Valliere et al., 2019, Valliere et al., 2020; Lange et al., 2016).

### Compositions

In an aspect of the invention, there is provided a pharmaceutical composition obtained from a method of the invention. Preferably the pharmaceutical composition is formulated with one or more pharmaceutically acceptable carriers known in the art.

The compositions described herein can be prepared for administration to a subject in need thereof orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form known in the art, subcutaneously, intravenously, intramuscularly, intraperitoneally, intrathecally, intraventricularly, intrasternally, and intracranial injection or infusion techniques.

Methods for preparing a cannabinoid into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for topical administration. The compositions for administration will commonly comprise a solution of cannabinoid or extract thereof dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of a cannabinoid or extract thereof of the present invention in these formulations can vary widely, and will be selected primarily based on fluid volumes, viscosities, body weight and the like in accordance with the particular mode of administration selected and the subject's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, a cannabinoid or extract thereof of the present invention can be administered in a manner compatible with the dosage formulation and in such amount as is therapeutically/prophylactically effective. Formulations are easily administered in a variety of dosage forms, such as the type of topical solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver a cannabinoid or extract thereof of the present invention.

### Dosages

Suitable dosages of a cannabinoid or extract thereof of the present invention will vary depending on the specific cannabinoid or extract thereof and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED50 of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC50 (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a cannabinoid, pharmaceutical composition or extract thereof described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of a cannabinoid or extract thereof sufficient to achieve the stated result in a subject.

The term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific cannabinoid or extract thereof administered and/or the particular subject and/or the type or severity or level of condition and/or predisposition (genetic or otherwise) to the condition. Accordingly, this term is not to be construed to limit the present invention to a specific quantity, e.g., weight or amount of a cannabinoid or extract thereof, rather the present invention encompasses any amount of the a cannabinoid or extract thereof sufficient to achieve the stated result in a subject.

### Medical Uses

It will be understood that the invention finds utility in the treatment of any disease or condition responsive to treatment with a cannabinoid or extract thereof described herein.

The disease or condition that may be treated and/or prevented using the cannabinoids of the invention include but are not limited to chronic pain, neuropathic pain, cancer, nausea and/or vomiting associated with cancer chemotherapy, lack of appetite, multiple sclerosis, spasticity associated with multiple sclerosis or spinal cord injury epilepsy, Parkinson's disease, anorexia and/or weight loss, irritable bowel syndrome, Tourette syndrome, amyotrophic lateral sclerosis, Huntington's disease, Dystonia, Dementia, Glaucoma, traumatic brain injury and/or intracranial haemorrhage, addiction, anxiety, depression, sleep disorders, post-traumatic stress disorder, microbial infection including methicillin-resistant Staphylococcus aureus (MRSA) and biofilm producing organisms, dermatitis, acne, schizophrenia and other psychoses. Additionally, any other disease or condition known in the art to be susceptible to cannabinoid treatment may be contemplated for treatment by the cannabinoids of the invention.

A subject in need of treatment may present a number of symptoms depending on the type of disease that the inflammation is associated with. In an aspect, a subject in need of treatment may exhibit well characterised symptoms associated with a disease or condition described herein.

The terms “treatment” or “treating” of a subject includes the administration of a a cannabinoid or extract thereof of the invention to a subject with the purpose of delaying, slowing, stabilizing, curing, healing, alleviating, relieving, altering, remedying, less worsening, ameliorating, improving, or affecting the condition or disease, or a symptom of the disease or condition.

The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury. In particular, treating refers to a reduction in a symptoms associated with the disease or condition.

The existence of, improvement in or treatment of the disease or condition may be determined by any clinically or biochemically relevant method known in the art. A relevant method may be measurement of symptoms. For example in the case of chronic pain, an improvement in symptoms may include reduced joint pain, reduced muscle aches, reduced burning pain, reduced fatigue, reduced sleep apnoea, reduced mood problems, including depression, anxiety, and irritability. Alternatively in the case of epilepsy, an improvement in symptoms may include reduced confusion, reduced staring spells, reduced stiff muscles, reduced uncontrollable jerking movements of the arms and legs, reduced loss of consciousness or awareness and reduced mood problems, including anxiety and fear. The improvement or treatment may be determined directly from the subject, or a sample or biopsy therefrom. The sample or biopsy may be of the diseased tissue.

### EXAMPLES

### Example 1—Materials and Methods

### Plant Materials

Olivetolic acid (OA; 2,4-dihydroxy-6-pentyl-benzoic acid) used in N. benthamiana infiltrations assays was purchased from Toronto Research Chemicals. Cannabigerolic acid (CBGA; 3-[(2E)-3,7-dimethyl-2,6-octadien-1-yl]-2,4-dihydroxy-6-pentyl-benzoic acid) was a Certified Reference Material (CRM) at 1 mg/mL in acetonitrile, purchased from Cerilliant (TX, USA), and stored at −80° C. Hexanoic acid (C6), geranyl-pyrophosphate (GPP as Trisammonium salt), LCMS-grade solvents (methanol, acetonitrile, isopropanol), formic acid, and ammonium acetate were purchased from Nu Chek Prep Inc (MN, USA) and ThermoFisher Scientific (MA, USA). Strata X-AW 33tm Polymeric Weak Anion columns (100 mg/6 mL; sorbent mass/volume) were purchased from Phenomenex Australia Pty Ltd (Sydney, Australia). Antibiotics and their concentrations used in A. tumefaciens growth medium include Rifampicin (25 μg/ml), Kanamycin (50 μg/ml), Carbenicillin (50 μg/ml) and Gentamycin (15 μg/ml).

### Genetic Constructs

All coding sequences were optimized using GeneArt (ThermoFisher Scientific Inc, MA, USA) online algorithm for expression in N. benthamiana, domesticated to ensure compatibility with the Golden Gate (GG) cloning system (Weber et al., 2011). Optimised coding sequences were flanked with appropriate 4 nucleotide overhangs according to Engler et al. (2014) (SEQ ID 5-14). The 2×35S promoter and Nos terminator level 0 GG modules correspond to pICH51288 and pICH41421, respectively (Engler et al., 2014). Modules were assembled into the pICH47732 backbone (Engler et al., 2014) by GG cloning to contain the optimised coding sequences for either CsAAE1, CsOLS, CsOAC, or CsCBGAS and resulting expression vectors were named pCAN1-3 and pCAN7 respectively (see Table 3).

The N terminus of Rubisco small subunit (SSU) was previously shown to redirect cytosolic proteins to the chloroplast in plants (Lee et al., 2002). A level 0 GG module containing the coding sequence for the Rubisco small subunit chloroplast targeting peptide was based on pICH78133 as described by Engler et al. (2014) with minor modifications (SEQ ID NO:15). Modules were assembled into the pICH47732 backbone (Engler et al., 2014) by GG cloning to contain the coding sequences for either CsAAE1, CsOLS, CsOAC, or CsCBGAS downstream of the Rubisco SSU targeting peptide and resulting expression vectors were named pCAN4-6 respectively (Table 3).

The sequence coding for the mNeonGreen fluorescent protein (Shaner et al., 2013) was designed as a GG level 0 module with flanking overhangs that allow for incorporation as a C-terminal fusion protein (SEQ ID 16). The mNeonGreen protein was chosen because it was previously shown to be a robust fluorescent reporter in plants and did not alter protein targeting (Stoddard and Rolland, 2019). Modules were assembled into the pICH47732 backbone (Engler et al., 2014) by GG cloning to contain the coding sequences for one of CsAAE1, CsOLS, CsOAC, and CsCBGAS between the Rubisco SSU targeting peptide and the fluorescent protein, resulting expression vectors were named pCAN11-13 (Table 3).

Three level 0 GG modules were designed to incorporate elements of the Bean yellow dwarf virus (BeYDV) to test if the recombinant protein expression levels could be elevated (Baltes et al., 2014). Module EN38510 contained the BeYDV long intergenic region (LIR), flanked by GGAG and TACT GG sequences at the 5′ and 3′ end, respectively (SEQ ID NO: 17). The 2×35S promoter and Tobacco Mosaic Virus (TMV) 5′ UTR sequence were designed as a GG module (EN38509) compatible with EN38510 by using TACT and AATG as the 5′ and 3′ GG linker sequences, respectively (SEQ ID NO:18). An alternative terminator module (EN38511) contained the 35S terminator sequence of pICH41414 (Engler et al., 2014), as well as the BeYDV short intergenic region (SIR), Rep/RepA coding sequences and LIR (SEQ ID NO: 19). An internal BsaI site was removed in the Rep protein and a single nucleotide substitution was introduced as per Diamos and Mason (2019). Modules were assembled into the pICH47732 backbone (Engler et al., 2014) by GG cloning to provide vectors containing the coding sequences for either CsAAE1, CsOLS, CsOAC, CsCBGAS, with module EN38510, module EN38509, and module EN38511. The resulting expression vectors were named pCAN31-37 (see Table 3).

Gene synthesis was outsourced to either GeneArt (ThermoFisher Scientific, MA, USA) or Twist Bioscience (CA, USA). The binary expression vector pICH47732 served as the backbone for all GG assembly reactions (Engler et al., 2014) and is referred to as the ‘standard vector’ herein. A list of all expression vectors can be found in Table 3 below.

TABLE 3
List of expression constructs

| | | Promoter + | N-terminal | | C-terminal | Terminator + |
|---|---|---|---|---|---|---|
| Vector | LIR | 5′UTR | fusion | ORF | fusion | 3′ UTR |
| pCAN1 | — | 2x35S | — | CsOAC | — | Nos |
| pCAN2 | — | 2x35S | — | CsAAE1 | — | Nos |
| pCAN3 | — | 2x35S | — | CsOLS | — | Nos |
| pCAN4 | — | 2x35S | Rubisco SSU | CsOAC | — | Nos |
| pCAN5 | — | 2x35S | Rubisco SSU | CsAAE1 | — | Nos |
| pCAN6 | — | 2x35S | Rubisco SSU | CsOLS | — | Nos |
| pCAN7 | — | 2x35S | — | CsCBGAS | — | Nos |
| pCAN11 | — | 2x35S | Rubisco SSU | CsOLS | mNeonGreen | Nos |
| pCAN12 | — | 2x35S | Rubisco SSU | CsOAC | mNeonGreen | Nos |
| pCAN13 | — | 2x35S | Rubisco SSU | CsAAE1 | mNeonGreen | Nos |
| pCAN31 | EN38510 | EN38509 | — | CsAAE1 | — | EN38511 |
| pCAN32 | EN38510 | EN38509 | — | CsOAC | — | EN38511 |
| pCAN33 | EN38510 | EN38509 | — | CsOLS | — | EN38511 |
| pCAN34 | EN38510 | EN38509 | — | CsCBGAS | — | EN38511 |
| pCAN35 | EN38510 | EN38509 | Rubisco SSU | CsAAE1 | — | EN38511 |
| pCAN36 | EN38510 | EN38509 | Rubisco SSU | CsOAC | — | EN38511 |
| pCAN37 | EN38510 | EN38509 | Rubisco SSU | CsOLS | — | EN38511 |
| pCAN38 | — | 2x35S | — | AtALP4.1 | — | Nos |
| pCAN39 | — | 2x35S | — | AtALP4.2 | — | Nos |
| pCAN40 | — | 2x35S | — | AtPLIP1 | — | Nos |
| pCAN41 | — | 2x35S | — | TomloxC | — | Nos |
| pCAN42 | EN38510 | EN38509 | — | AtALP4.1 | — | EN38511 |
| pCAN43 | EN38510 | EN38509 | — | AtALP4.2 | — | EN38511 |
| pCAN44 | EN38510 | EN38509 | — | AtPLIP1 | — | EN38511 |
| pCAN45 | EN38510 | EN38509 | — | TomloxC | — | EN38511 |

N. benthamiana Leaf Infiltration Assay

Agrobacterium tumefaciens GV3101 cultures containing individual expression vectors were grown in 10 mL LB medium, supplemented with antibiotics. Following incubation for 2 days at 28° C., acetosyringone was added to the cultures at a final concentration of 100 μM. Cultures were incubated for 3 hours at 28° C. and collected by centrifugation at 4000 rpm for 5 minutes. Cell pellets were gently resuspended in an equal volume of infiltration buffer (5 mM MgSO₄, 5 mM MES, 5% Ethanol, pH 5.7), containing 100 μM acetosyringone, 0.1% Tween20 and appropriate substrates (C6, GPP, OA) as described below. The resuspended cultures were mixed to obtain the desired combination of different expression vectors with the OD₆₀₀ of each culture in the final infiltration solution set to 0.125. P19 was included as a viral suppressor protein in all infiltrations, as described by Wood et al. (2009).

Cell solutions were infiltrated into the underside of N. benthamiana leaves using a 1 mL syringe, in combination with C6, GPP, OA and/or CBGA as substrates and/or positive controls. Infiltrated substrates that were individually and/or co-infiltrated included 1 mM GPP, 2 mM C6, 0.5 mM OA, or 0.2 mM CBGA. Infiltrated leaf areas were marked with a pen and plants were placed in cabinet (Conviron PGC20Flex; 23° C.; 16 h/8 h light/dark cycle). Three days after the initial infiltration, marked leaf areas were re-infiltrated with infiltration buffer containing appropriate substrates. Infiltrated leaf spots (˜400 mg fresh weight) were harvested 4 days post-gene infiltration. Samples were immediately flash frozen in liquid N₂ and stored in −80° C. until extraction. Negative controls consisted of either A. tumefaciens expressing the viral suppressor protein P19 (‘P19’), infiltration buffer without any A. tumefaciens cultures (‘Inf neg’) or a buffer control during extraction (‘Blank’).

### Extraction of OA and CBGA

Cannabinoids were extracted from frozen infiltrated N. benthamiana leaf samples by adding 800 μL 20% MEOH (prepared in ultra-pure water) in 2 mL Eppendorf tubes. Samples were lysed using a Qiagen Tissuelyzer (3 min at 29.0 Hz) (Qiagen N.V., Hilden, Germany) with two stainless steel ball bearings. Following sonication for 10 min in an ice bath, samples were immediately centrifuged for 5 min at 13,000 RPM. The extraction procedure was repeated twice and pooled. The combined extracts were filtered using a 0.22 μm nylon microcentrifuge tube filter spun at 12,000 RPM for 2 min. A total of 10 or 6 replicate extracts were combined in case of the infiltration experiments with the standard or BeYDV vector, respectively. Next, samples were transferred to 10 mL glass vials, dried under N₂ and re-suspended in equal volume of 100 mM ammonium acetate buffer (pH 4.9±0.5) buffer solution, followed by solid-phase extraction purification.

Strata X-AW 33 μm cartridges (Phenomenex, Sydney Australia) were preconditioned and equilibrated by adding 6 mL acetonitrile and 6 mL of 100 mM ammonium acetate buffer (pH 4.9). Pooled samples were loaded on the column, followed by washing steps with 6 mL of 100 mM ammonium acetate (pH 4.9) and 6 mL methanol. Samples were dried for 5 min under vacuum before elution with 6 mL of ammonium hydroxide:acetonitrile (5:95, v/v). The eluates were dried under a stream of N₂ at 30° C. and resuspended in acetonitrile, corresponding to 1/10 or 1/20 of the initial loading volume in case of the infiltration experiments with the standard and BeYDV-derived vectors, respectively. A 50 μL aliquot was taken for immediate LC-MS analysis while the remainder of the eluate was stored at −80° C.

### LC-MS Detection of OA and CBGA

Chromatographic separation of OA, CBGA and their glycosylated forms was achieved using a Vanquish UPLC system equipped with a thermostatted autosampler and column compartment on an Agilent Zorbax Eclipse Plus C18 Rapid Resolution HD column (2.1×150 mm; 1.8 μm particle size) (Agilent CA, USA) coupled with a guard-column under constant temperature of 40° C. Mobile phase A consisted of 0.1% formic acid in ultra-pure water and mobile phase B consisted of 0.1% formic acid in acetonitrile. The gradient separation started with isocratic hold at 5% B for 2 min, followed by a linear gradient of 67% B at 5 min and 65% B at 9 min, reaching 100% B at 18 min and maintained for 2 min. Re-equilibration to the initial conditions was achieved by holding at 5% B for 2 min before the next injection. The flow rate was set at 0.3 mL/min using a UHPLC Vanquish binary pump (Thermo Scientific, MA, USA). Aliquots of 2 μL sample volume including experimental negative and positive controls, procedural blanks, instrumental blanks and standards (OA, CBGA) were injected.

OA and CBGA and their glycosylated forms were detected on an Orbitrap Q-Exactive (Thermo Scientific, CA, USA) mass spectrometer equipped with heated electrospray ionization (HESI) source operated in positive ionization mode. The capillary and vaporizer temperatures were 256° C. and 412° C., respectively, electrospray voltage of 3.5 kV, sheath gas 48 and auxiliary gas 11 (arbitrary units), S-lens RF level of 50. Normalized collision energy was optimized at values of 15 and 25. Data acquisition was set using parallel reaction mode at a resolving power of 70,000 FWHM at m/z 200. Mass calibration was performed before runs using Pierce-™ LTQ ESI positive ion calibration solution (Thermo Scientific™, San Jose, CA, USA). Detection was based on calculated [M+H]⁺ molecular ions with an accuracy of 2 ppm and retention time of target compounds present in the inclusion list.

Mass calibration was performed before runs using Pierce™ LTQ ESI positive ion calibration solution (Thermo Scientific™, San Jose, CA, USA). Detection was based on calculated [M⁺H]⁺ molecular ions with an accuracy of 2 ppm and retention time of target compounds present in the inclusion list.

Instrumental control and data acquisition were achieved using Thermo Scientific Xcalibur (Version 4.3.73.11, 2019) (Thermo Scientific, CA, USA) and data processing was performed with Freestyle software. OA and CBGA in glycosylated and single forms were identified by comparison of MS spectra and fragmentation patterns to those of reference standards and/or infiltrated leaves.

Serial dilutions from working standard solutions were prepared for OA and CBGA at concentrations from 0.0025 to 1 μg/mL. Calibration curves were constructed by using the peak areas for each OA (m/z 225.111) and CBGA (m/z 343.2268). External standard linear calibration curve obtained a R²≥0.9998. Intra-day injection precision was <4% CV (n=5). Instrument detection limit (LOD) of 9 μg L₋⁻¹ for CBGA and 11 μg L-₋⁻¹ for OA and were calculated based on the standard deviation of the response and slope.

Glycosylated forms of OA (OA-Glc) and CBGA (CBGA-Glc) were obtained by infiltrating leaves separately with 0.5 mM of OA and 0.2 mM of CBGA followed by sample extraction as previously described. Acquired data (area of chromatograms) was corrected for sample fresh weight and dilution/concentration factors during extraction.

### Confocal Microscopy

To validate correct retargeting of the designed enzymes to the chloroplast in N. benthamiana, the coding sequences for the Rubisco SSU chloroplast targeting signal and the mNeonGreen fluorescent protein were cloned in frame at to the N- and C-termini of the CsAAE1, CsOLS and CsOAC, respectively. mNeonGreen fusion constructs containing the hemagglutinin epitope (EN38113; SEQ ID 28) instead of the Rubisco SSU served as controls to visualize cytosolic localization. The A. tumefaciens GV3101 cultures were infiltrated in N. benthamiana leaves as described above in the absence of any substrates. After three days, leaf discs of approximately 0.5 cm in diameter were collected from infiltrated leaves. The abaxial side of these discs was imaged with a Leica SP8 confocal laser-scanning microscope (Leica Microsystems, Sydney, Australia) equipped with a 40× (NA=1.1) water immersion objective and the Leica Application Suite (LAS-X, Leica Microsystems, Sydney, Australia). Image z-stacks were collected using HyD detectors and sequential scanning in separate tracks. In the first track, tissue was excited at 506 nm and mNeonGreen signal was collected at 512-530 nm together with transmitted light. The mNeonGreen detector was gated between 0.3 ns and 6 ns to remove cell wall autofluorescence. In the second track, tissue was excited at 633 nm and chloroplast autofluorescence was collected at 650-690 nm.

### RNA Expression

For quantification of RNA expression levels via digital droplet PCR in tobacco, the methods described in Vanhercke T., et al., (2017) were followed.

### Protein Expression

Protein expression levels can be quantified by Western analysis using HA-tag or mNeonGreen fusion proteins and antibodies against HA epitope or mNeonGreen fluorescence. Alternatively, absolute quantification of the expressed protein levels in leaves can be achieved by a targeted LC-MS/MS method (Colgrave et al., 2019).

### Example 2—the Biosynthesis Pathway of OA and CBGA

Different intracellular localization patterns of the cannabinoid biosynthetic enzymes suggest a model, starting with the production of OA from malonyl-CoA and C6 in the cytosol of C. sativa trichome cells (FIG. 1). OA is subsequently transported to the chloroplast for further conversion to CBGA by the CsCBGAS. As the final step, CBGA is secreted into the apoplastic storage cavities of the trichome heads where it is converted to THCA, CBDA and minor cannabinoids such as CDCA (Andre et al., 2016; Gülck and Moller, 2020; Arif et al., 2021).

Cannabinoid biosynthesis enzymes are recognised to act on alternate substrates. FA chain length promiscuity of cannabinoid biosynthesis enzymes was shown in yeast feeding experiments by Luo et al., (2019).

C. sativa acyl activating enzyme (CsAAE1; AFD33345.1; SEQ ID NO:1) is a cytosolic CsAAE1 enzyme present in the trichome. It is a trichome-specific acyl-CoA synthetase that converts C6 in its CoA derivative. CsAAE1 is a member of the acyl-activating enzyme (AAE) superfamily that activate carboxylic acids through an adenylate intermediate (Stout et al., 2012). This family of enzymes all possess a well-conserved 12 amino acid residue AMP-binding motif (PROSITE PS00455) (Shockey and Browse, 2011). The AAE1 belongs to the EC6.2.1.2 classification. CsAAE1 is known to accept FA that have different chain lengths (Stout et al., 2012).

C. sativa olivetol synthase (CsOLS; BAG14339.1; SEQ ID NO:2) is one of two enzymes working in concert to produce OA from C6-CoA and malonyl-CoA. Taura et al., (2009) identified the CsOLS gene as the polyketide synthase in C. sativa trichomes that condenses C6-CoA and a variety of other short-chain fatty acids together with malonyl-CoA. CsOLS can accept C4-C8 CoA substrates (Taura et al., 2009). CsOLS is a cytosolic protein (Stout et al., 2012) and its crystal structure has been resolved recently (Kearsey et al., 2020). Non-natural variant OLS mutants have been recently described in WO2020/214951.

C. sativa olivetolic acid cyclase (CsOAC; AFN42527.1; SEQ ID NO:3) is the second enzymatic step needed to produce OA. CsOAC is localized in the cytosol (Gagne et al., 2012). It refers to a 3,5,7-trioxododecanoyl-CoA cyclase or a 3,5,7-trioxundecanoyl-CoA cyclase (EC4.4.1.26) this enzyme class is capable of converting 3,5,7-trioxododecanoyl-CoA into OA or 3,5,7-trioxundecanoyl-CoA into divarinolic acid.

C. sativa CBGA synthase (CBGAS; DAC76710.1; SEQ ID NO:4). In Cannabis OA is transported into the chloroplast for further conversion to CBGA by the CsCBGAS. Recently, both Luo et al. (2019) and Gulck et al. (2020) identified CsPT4 as the functional CBGAS prenyltransferase in C. sativa. Unlike CsOLS and CsOAC, CsCBGAS is a membrane-bound enzyme residing in the chloroplast (Gulck et al., 2020). An alternative prenyltransferase NphB from Streptomyces sp. strain CL190, capable of producing CBGA was identified by Valliere et al. (2019). This group used structure-based protein design to improve the non-specific CBGAS activity of the soluble NphB. The CBGAS refers to a cannabigerolicacidsynthase (EC 2.5.1.102) capable of converting GPP and Olivetolic acid (OA) or GPP and divarinolic acid (DVA) into cannabigerolicacid (CBGA) or cannabigerovarinic acid (CBGVA).

C. sativa THCA and CBDA synthases (THCAS; CBDAS). The final step in the cannabinoid biosynthesis pathway consists of the conversion of CBGA into THC or CBD as the major cannabinoid end-products by the CsTHCAS or CsCBDAS, respectively. In C. sativa both enzymes are located in the apoplast, explaining the accumulation of the major cannabinoids such as THC and CBD at very high levels in the extra cellular storage globules (Sirikantaramas et al., 2004; Taura et al., 2007). CBDA synthase (EC1.21.3.8) is capable of converting CBGA or CBGVA into cannabidiolic acid (CBDA) or cannabidivarinic acid (CBDVA). CBDAS can accept CBGA (C5 tail) and CBGVA (C3 tail) in vitro (Valliere et al., 2019). THCA synthase (tetrahydrocannabinolic acid synthase EC1.21.3.7) is able to convert CBGA or CBGVA into THCA (tetrahydrocannabinolic acid) or THCVA (tetrahydrocannabivarinic acid).

### Example 3—Functional CBGA Biosynthesis Pathway Operating in the Cytosol and Chloroplast

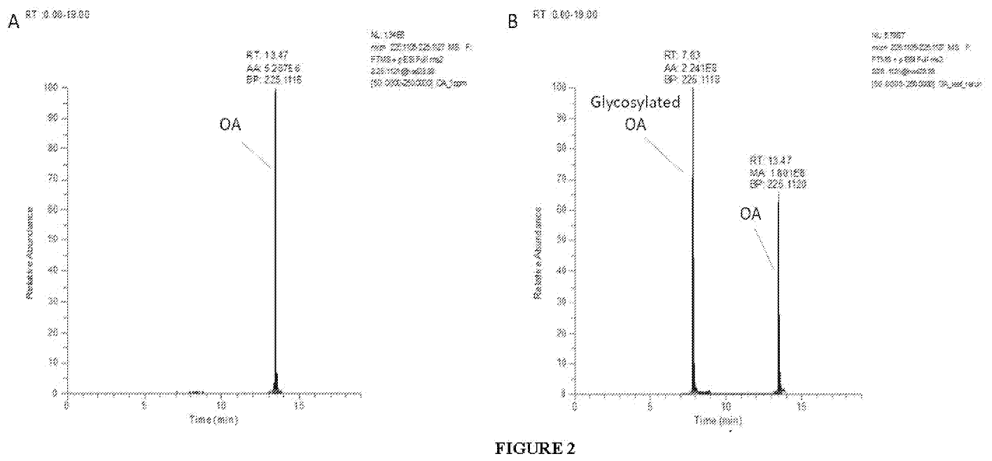

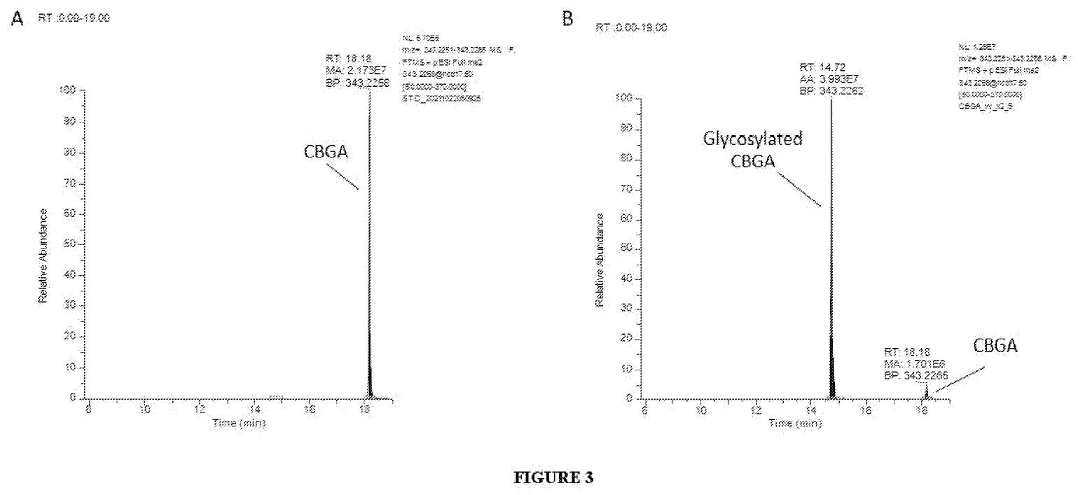

Infiltration of OA or CBGA in N. Benthamiana revealed rapid glycosylation of both molecules, confirming similar observations by Gulck et al. (2020) (FIGS. 2 and 3).

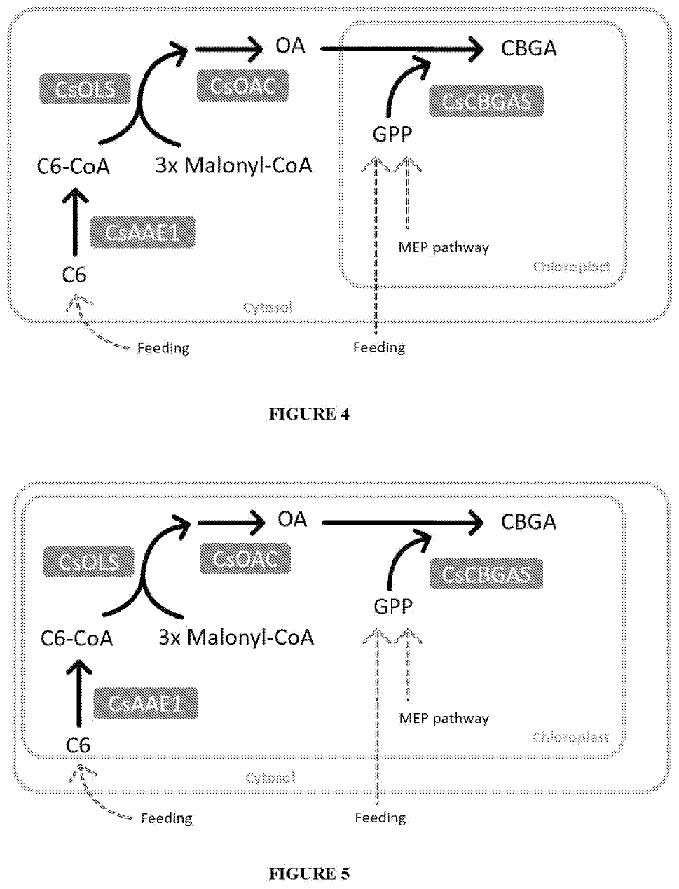

The present inventors aimed to build the CBGA biosynthesis pathway of C. sativa in N. benthamiana leaves by transforming the enzymes in the biosynthetic pathway using the vectors described in Example 1. The enzymes for synthesising OA were expressed in the cytosol and the CBGA biosynthetic enzymes targeted the chloroplast, respectively (FIGS. 1 and 4). Since CsAAE1, CsOLS and CsOAC are localized in the cytosol in the trichomes of C. sativa, the authors expressed codon optimized versions of the three genes in N. benthamiana without any additional targeting peptides (pCAN1-3). In C. sativa, CsCBGAS is localized in the chloroplast membrane as a result of a targeting peptide present within the naturally occurring CBGAS sequence. To achieve chloroplast localization of recombinant CsCBGAS in N. benthamiana, the inventors expressed a codon optimized version while keeping its own native plastidial targeting sequence (pCAN7).

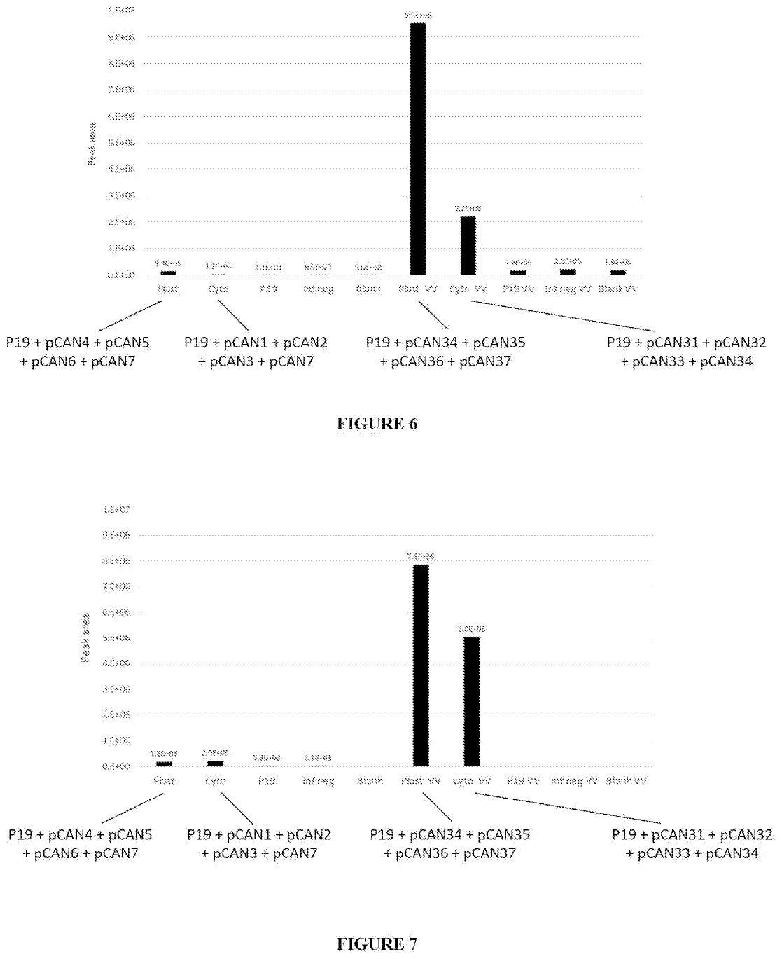

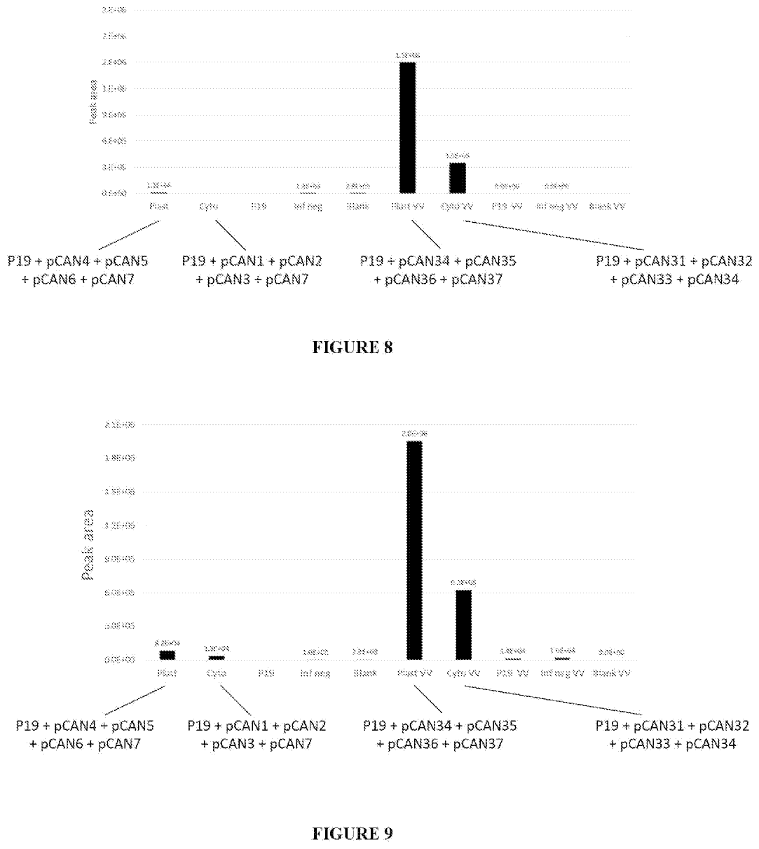

When N. benthamiana was infiltrated with the vectors pCAN1-3 and pCAN7 in the presence of C6 and GPP as substrates, low levels of OA were detected that were absent in the corresponding P19 and blank infiltrations, serving as negative controls (FIG. 6). The glycosylated forms of both OA and CBGA were also detected at low levels, indicating a functional recombinant CBGA biosynthesis pathway (FIGS. 7 and 9). CBGA in its unmodified form however was not detected (FIG. 8). This observation is in accordance with the intrinsic rapid glycosylation of CBGA in N. benthamiana leaves.

### Example 4—Functional CBGA Biosynthesis Pathway Targeted to the Chloroplast

The functional pathway described in Example 3 relies on the biosynthesis of OA in the cytosol, followed by transport of this cannabinoid precursor into the chloroplast for further conversion into CBGA by the membrane bound CsCBGAS. The inventors hypothesised that, unlike in C. sativa trichomes, the transport of OA into the chloroplast in N. benthamiana might pose a metabolic bottleneck reducing the production of CBGA. In addition, glycosylation of OA could lead to a further metabolic ‘dead-end’ if the CBGAS enzyme is unable to recognize OA in a glycosylated form as a substrate. To improve the flux of OA to CBGA, the inventors designed an alternative CBGA biosynthetic pathway that co-locates all enzymes into the chloroplast. It was proposed that co-locating all biosynthesis steps in the same organelle could lead to better availability of the OA substrate for the CsCBGAS enzyme (FIG. 5). The inventors therefore re-targeted each of the CsAAE1 (pCAN5), CsOLS (pCAN6) and CsOAC (pCAN4) to the chloroplast by including a targeting peptide of the Rubisco SSU as an N-terminal fusion.

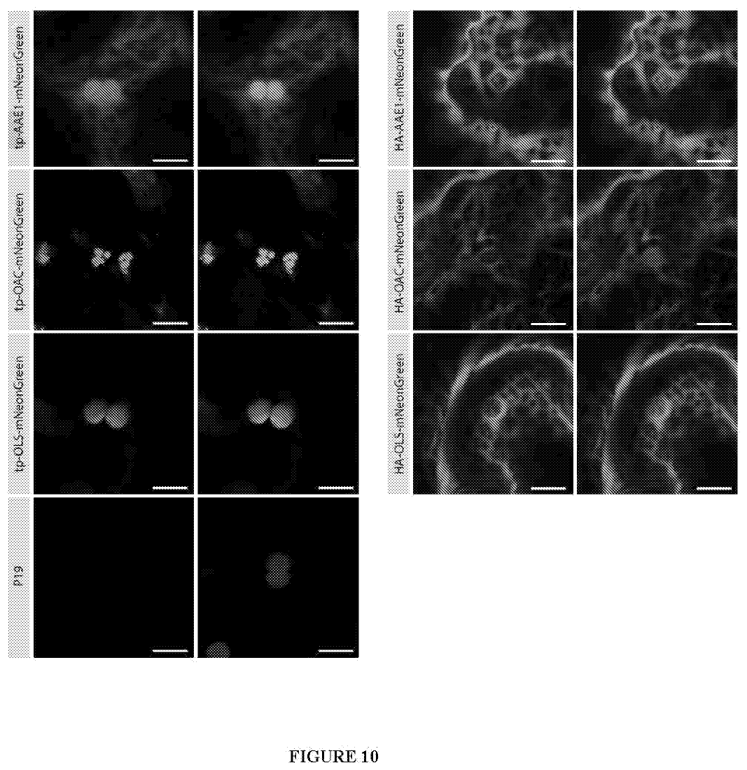

Chloroplast targeting of CsAAE1, CsOLS, and CsOAC containing the N-terminal Rubisco SSU peptide was confirmed by confocal microscopy (FIG. 10). The re-targeted CsAAE1 protein also showed some cytosolic localization. In the absence of a chloroplast targeting peptide, all three proteins resided in the cytosol as expected (FIG. 10).

When N. benthamiana was infiltrated with the vectors pCAN4-6 and pCAN7 in the presence of C6 and GPP as substrates, both OA and CBGA as well as their glycosylated derivatives were produced at detectable levels (FIGS. 6-9). The plastidial targeted pathway successfully produced detectable levels of CBGA and the glycosylated CBGA was substantially higher compared to the cytosolic-chloroplast hybrid pathway of Example 3, demonstrating an improved flux by re-targeting all enzymatic steps to the chloroplast. CBGA is the key cannabinoid end product of the pathway assembled in the N. benthamiana, the plastid pathway produced approximately 4 fold more glycosylated OA when compared to the cytosol-chloroplast model of Example 3. CBGA was detectable in the glycosylated form in the cytosol-chloroplast model of Example 3 at low levels which were dramatically increased (estimated about 24-fold increase) in the plastid targeting pathway. Furthermore unglycosylated CBGA was detected in the plastid targeting pathway described in this Example.

Chloroplast targeting remains complicated as based on the confocal microscopy experiments, as whilst both OLS and OAC relocated to the chloroplast, they appear to have different localization patterns within the chloroplast, despite the Rubisco chloroplast targeting peptide being fused to each sequence. Also, AAE1 fused with the same chloroplast targeting peptide still shows some localization in the cytosol. Nonetheless, the present inventors have obtained for the first time a fully demonstrated and functional CBGA biosynthetic cannabinoid pathway in a non-Cannabis host plant.

### Example 5—Enhanced Expression of a Functional CBGA Biosynthesis Pathway Operating in Cytosolic and Chloroplasts Using Deconstructed Viral Expression Vector

Several methods are known to increase expression of exogenous proteins in plants, including increasing the level of transcription using viral promoters, (eg CaMV 35 S promoter, maize-ubiquitin-1 promoter or the Agrobacterium tumefaciens nopaline synthase (NOS) promoter) suitable to the specific system. Terminators such as Ext terminator have been found to also improve protein expression. Plant viruses to boost protein expression are an alternative mechanism, particularly for transient expression. One such virus is the bean yellow dwarf virus (BeYDV), a Mastrevirus of the Geminiviridae family. The BeYDV contains a single-stranded circular DNA genome and uses a rolling circle mechanism to replicate its genome, it has been shown to result in a high production of gene copies and thereby increased protein expression.

To test if the recombinant protein levels in N. benthamiana could be increased and improve cannabinoid production, a series of level 0 modules, compatible with GG cloning and containing elements of the Bean yellow dwarf virus (BeYDV) as described in detail in Example 1 were designed. Cloning of CsAAE1 (pCAN31), CsOLS (pCAN33), CsOAC (pCAN32) and CsCBGAS (pCAN34) in this new expression system, N. benthamiana leaves were infiltrated as described in Example 1 in the presence of C6 and GPP as substrates.

Compared to infiltrations using a standard expression vector (Example 3), the use of the BeYDV-adapted vector system resulted in surprising and considerable increases in the levels of OA, CBGA and the glycosylated forms of OA and CBGA (FIGS. 6-9 and 10). It was estimated that the OA production was increased approximately 65 to 70 fold as a result of the cytostol-chloroplast expression model under the control of the viral vector expression system. In this experiment the glycosylated form of the OA was increased about 25 fold in the cytostol-chloroplast model under control of viral vector replication machinery. CBGA was detected successfully and CBGA in the glycosylated form was observed to be at a level about 19 fold greater than when compared to the results from Example 3. As expected, higher levels of the glycosylated forms (FIGS. 7 and 9) were observed, confirming earlier results described in Example 3.

### Example 6—Enhanced Expression of a Functional CBGA Biosynthesis Pathway Targeted to the Chloroplast Using Deconstructed Viral Expression Vector

Since re-targeting of the pathway in the chloroplast (Example 4) and the use of BeYDV genetic elements to increase expression (Example 5) resulted in increased levels of OA and CBGA, the combination of both strategies was undertaken to attempt to further increase the CBGA yields and metabolic flux through the recombinant cannabinoid pathway in N. benthamiana. To this end, the Rubisco SSU chloroplast targeting sequence was cloned in frame with the CsAAE1 (pCAN35), CsOLS (pCAN37), and CsOAC (pCAN36) in the new BeYDV-adapted expression system (see Table 3).

When N. benthamiana was infiltrated with the vectors pCAN35-37 and pCAN34 in the presence of C6 and GPP as substrates as described in Example 1, dramatically increased levels of OA, CBGA and their glycosylated derivatives were observed (FIGS. 6-9 and 10-11). Similar to earlier results obtained with a standard expression vector (Example 4), levels of all four cannabinoids were higher compared to the cytosolic-chloroplast pathway using the same expression system (Example 5). It was estimated that the OA production was increased approximately 65 to 70 fold as a result of the viral vector expression system. In this experiment the glycosylated form of the OA was increased about 40-45 fold in the chloroplast model under control of viral vector replication machinery. CBGA (not glycosylated) was detected at around 10-13 fold higher levels and CBGA in the glycosylated form was observed to be at a level about 2-3 fold greater than when compared to the results from Example 4.

Again, this confirms improved function of the CBGA biosynthesis pathway when all enzymes are located within the same subcellular compartment such as the chloroplast.

The inventors next sought to compare expression levels in one experiment using four independent biological replicates to allow for comparison between (1) cytosolic vs. chloroplast targeting, or (2) standard binary expression vector vs. deconstructed viral expression vector. The same constructs used in the previous experiments were used here for the four combinatorial gene infiltrations as follows: pCAN1+pCAN2+pCAN3+pCAN7 for cytosolic localization using binary vector (‘Cyto’); pCAN4+pCAN5+pCAN6+pCAN7 for plastidial targeting using binary vector (‘Plast’); pCAN31+pCAN32+pCAN33+pCAN37 for cytosolic localization using deconstructed viral vector (‘Cyto VV’); and pCAN34+pCAN35+pCAN36+pCAN37 for plastidial targeting using deconstructed viral vector (‘Plast VV’).

Nicotiana benthamiana infiltrations with Agrobacterium strains carrying expression vectors was performed as described in Examples 3-6. Each infiltration was combined with an Agrobacterium strain expressing the viral suppressory protein p19, while Agrobacterium strains expressing p19 and GFP were used as negative control. Substrates C6 and GPP were exogenously supplied as described in Examples 3-6.

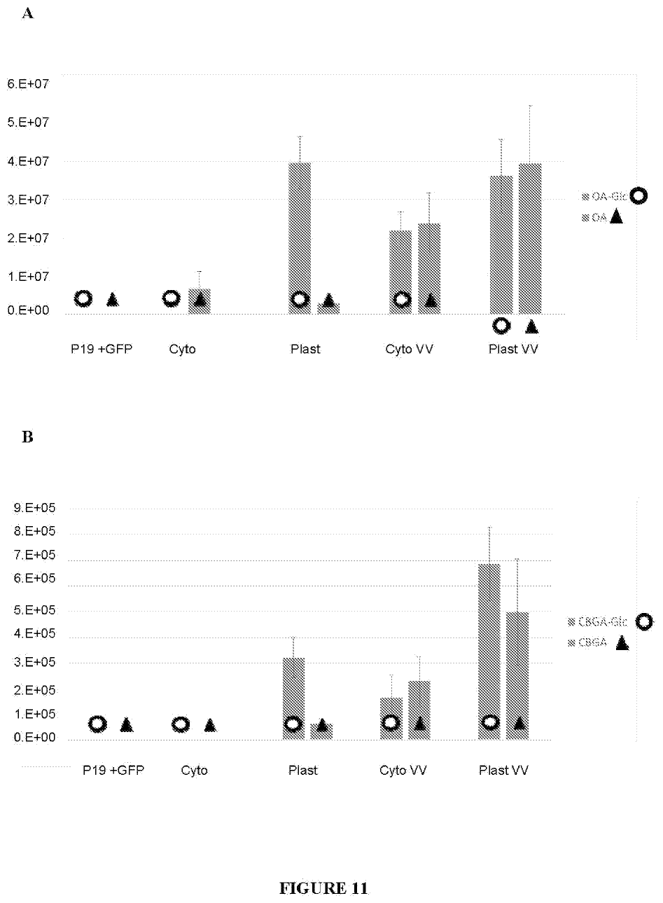

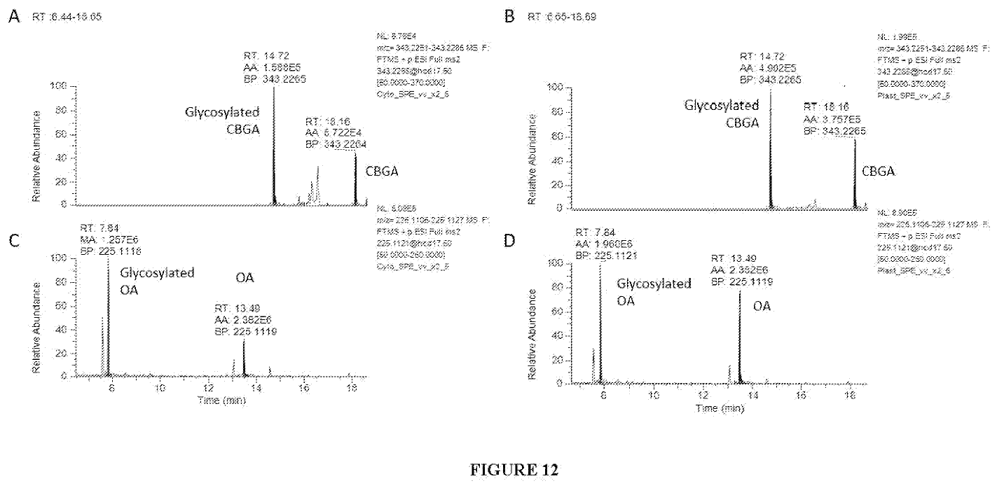

The results shown in FIG. 11 confirmed higher accumulation of OA and CBGA as well as their glycosylated derivatives when the recombinant proteins were targeted to the chloroplast, compared to localization in the cytosol. Higher yields of OA and CBGA were also detected in both cytosol and chloroplasts when transgenes were expressed using the deconstructed viral vector compared to normal binary expression vector.

Based on these results, the inventors propose approaches for the biosynthesis of CBGA in plant biomasses, whereby it it possible to yield surprisingly synergistic effects using cytostol-chloroplast and/or chloroplast targeted biosynthesis pathways boosted by the use of the viral vector replication machinery from a geminivirus such as BeYDV.

### Example 7—C6 Engineering

Plastid targeting of the OA biosynthesis enzymes CsAAE1, CsOLS and CsOAC were shown to successfully produce OA, although at a low level and mostly glycosylated with glucose in N. benthamiana leaves (Example 4). Previous infiltration experiments herein relied on exogenously supplied C6:0 as a substrate for CsAAE1 to produce hexanoyl-CoA, which is subsequently converted into OA by the combined action of CsOLS and CsOAC. Plant leaves normally accumulate medium chain length FAs mostly with carbon chain length of C16 or C18, while levels of C14 or shorter chain fatty acids are low. Very short chain length FAs (i.e. less than 10 carbon chain length), either in acyl-CoA form or esterified to a glycerol backbone, are rare in leaf. Increased release of C6 in the plastid could possibly enhance the OA synthesis by recombinant CsAAE1, CsOLS and CsOAC biosynthesis enzymes.

Two approaches were designed to test the possibility of increasing the accumulation of C6 in N. benthamiana leaves, combining with the expression of CsAAE1, CsOLS and CsOAC thereby to enhance OA synthesis. The first approach was designed to test the possibility of increasing the accumulation of cellular C6 content in N. benthamiana leaves, combined with the expression of CsAAE1, CsOLS and CsOAC to enhance levels of OA, without exogenous supply of C6 substrate. Arabidopsis thaliana acyl-lipid thioesterase (AtALT4, At1g68280) has been shown to result in the accumulation of short chain fatty acids (SCFAs) that contain 6 or 8 carbons in leaves and seeds (Kalinger et al., 2021). Transient expression of AtALT4 in N. benthamiana leaf also led to C6 fatty acid accumulation (Kalinger et al., 2021). AtALT4 contains a 47 amino acid transit peptide and is predicted to be localised in the chloroplast. There are two reported sequence versions of AtALT4 in NCBI, NP_001319347 and F4HX80, due to the intron prediction, designated AtALT4.1 and AtALT4.2 respectively.

A second approach is to express the A. thaliana Plastid Lipase1 (AtPLIP1, At3g61680, AC149785) and tomato 13-lipoxygenase (TomLoxC, AAB65766). AtPLIP1 hydrolyzes C18:2 or C18:3 from sn-1 position of a unique chloroplast-specific phosphatidylglycerol that contains 16:1^{Δ3trans} as its second acyl group (Wang, 2017). AtPLIP1 is a peripheral thylakoid membrane protein localised in chloroplast. TomloxC is also predicted to be chloroplast localized and to encode 13-lipoxygenase enzyme that produces C5 and C6 fatty acid-derived volatiles from the peroxidation of C18 polyunsaturated fatty acid in tomato leaf (Shen et al. 2014). Lipoxygenase activity is also thought to generate C6 from C18:2 in C. sativa (Gülck and Moller, 2020).

The coding sequences for AtALT4.1 (NP_001319347), AtALT4.2 (F4HX80), AtPLIP1 (AC149785) and TomloxC (AAB65766, SEQ ID NOs:20-23, respectively) were codon optimised for N. benthamiana and synthesised. The codon optimised nucleotide sequences are listed as SEQ ID NOs: 24-27. These coding sequences were cloned into Golden Gate cloning vector pICH47732 under 2×35S promoter as described in Example 1. The resulting expression vectors were named pCAN38-41 (Table 3). The same sequences were also cloned into Golden Gate cloning vector pICH47732 using modules EN38510, EN38509, and EN38511 as described in Example 1. The resulting expression vectors were designated pCAN42-45 (Table 3). These expression plasmids can be transformed into A. tumefaciens GV3103, and the resulting strains combined for N. benthamiana leaf infiltration as described in Example 1. The total leaf FA are analysed by gas chromatography (GC) to detect the accumulation of short chain FA, especially C6. Expression of AtALT4.1, AtALT4.2 or AtPLIP1+TomloxC can also be combined with the expression of CsAAE1, CsOLS and CsOAC to produce OA without C6 feeding.

Results from experiments conducted in N. benthamiana showed that the codon optimized sequence for AtALT4.1 (NP_001319347, SEQ ID NO: 24) was functional and led to the production of C6. AtALT4.2 showed activity supporting C6 production. AtALT4.1 was used in the following experiments, referred to as AtALT4.

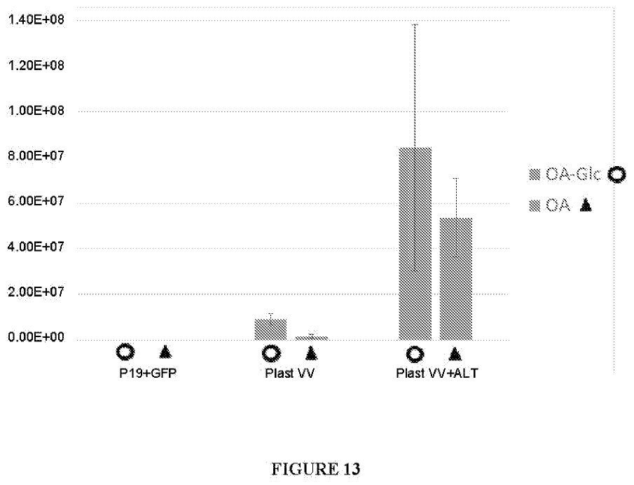

AtALT4 (NP_001319347) was heterologously expressed in N. benthamiana leaves, using a deconstructed viral expression vector combined with the chloroplast targeting version of OA biosynthesis pathway (CsAAE1, CsOLS and CsOAC) from deconstructed viral expression vectors. The result showed that OA biosynthesis pathway combined with AtALT4 produced a larger amount of OA without C6 feeding (FIG. 13, Plast VV+ALT), when compared to expressing the OA biosynthesis pathway alone with an exogenous C6 substrate supply (FIG. 13, Plast VV).

### Example 8—Stable Tobacco Transformation

Codon optimized sequences of CsAAE1, CsOLS, CsOAC, and CsCBGAS are cloned into a plant binary expression vector such as, pORE04 (Coutu et al., 2007). To achieve subcellular localization of CsAAE1, CsOLS, and CsOAC in either the cytosol or chloroplast, all three genes are cloned either in the absence of a targeting peptide or cloned in frame with a chloroplast targeting peptide such as the Rubisco SSU described in Examples 1 and 4. Promoters driving transgene expression are constitutive (e.g. 35S, enTCUP2), tissue specific (e.g. Rubisco SSU), developmentally regulated (e.g. SAG12), inducible or a combination thereof. A. tumefaciens-mediated transformation of N. tabacum and tissue culture regeneration is undertaken as described in Vanhercke et al. (2014). Confirmed transgenic events are transferred to the glasshouse and analyzed for increased CBGA levels after infiltration with C6 and GPP substrates.

The same combination of transgenes, promoters and subcellular targeting peptides as described above are cloned into a plant expression vector, containing viral genetic elements to increase expression similar as, but not limited to, Zhang and Masson (2006) or Dugdale et al., (2014). Confirmed transgenic events are transferred to the glasshouse and analyzed for increased CBGA levels after infiltration with C6 and GPP substrates.

The same set of transgenes as described above are cloned into a chloroplast transformation vector and transplastomic N. tabacum lines and tissue culture regeneration are generated (Svab and Maliga, 1993). Confirmed transgenic events are transferred to the glasshouse and analyzed for increased CBGA levels after infiltration with C6 and GPP substrates.

The same set of transgenes are cloned together with the AtALT4, AtPLIP and TomLoxC, or AtALT4 and AtPLIP and TomloxC into a plant expression vector. Promoter, subcellular targeting and vector backbones are the same as described above. A. tumefaciens-mediated transformation and tissue culture regeneration of N. tabacum is undertaken essentially as described in Vanhercke et al. (2014). Confirmed transgenic events are transferred to the glasshouse and analyzed for increased CBGA levels in the absence of exogenous C6 substrate in both vegetative (e.g. leaf) tissues and seed. The CsAAE1, CsOLC, CsOAC, and CsCBGAS are cloned together with the AtALT4, AtPLIP and TomLoxC, or AtALT4 and AtPLIP and TomloxC in a chloroplast transformation vector. Transplastomic N. tabacum lines are generated essentially as described by Svab and Maliga (1993). Confirmed transgenic events are transferred to the glasshouse and analyzed for increased CBGA levels in the absence of exogenous C6 substrate in both vegetative (e.g. leaf) tissues and seed.

The present invention is the first demonstration of a functional CBGA cannabinoid biosynthesis pathway in a non-Cannabis plant host. In particular, co-expression of CsAAE, CsOLS, CsOAC, and CsCBAS genes codon optimized for N. benthamiana results in the synthesis of OA and the glycosylated forms of OA and CBGA. We further show that re-targeting of the CsAAE1, CsOLC and CsOAC to the chloroplasts of N. benthamiana cells further improves yields of OA, CBGA and their respective glycosylated forms. Dramatic increases in the levels of all four cannabinoids were obtained when the recombinant cytosolic/chloroplast and chloroplast CBGA biosynthetic pathways were overexpressed using an improved vector that incorporated specific BeYDV genetic elements. Again, levels of OA, CBGA and their glycosylated derivatives were increased when all biosynthetic genes were co-located in the chloroplast.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from Australian provisional patent application AU 2021904150, filed 20 Dec. 2021, the entire contents of which is incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Alharbi (2020) Epilepsy & Behavior 112:107452.
Andre et al. (2016) Frontiers in Plant Science 7:19.
Arif et al. (2021) Plants 10:1307.
Ausubel et al. (editors), Current Protocols in Molecular Biology, Greene Pub.
Associates and Wiley-Interscience (including all updates until present),
Baltes et al. (2014) Plant Cell 26:151-163.
Baron (2018) Headache 58:1139-1186.
Begemann et al. (2017) Science Reports 7:1-6.
Bevan et al. (1983) Nature 304:184-187.
Bevan (1984) Nucleic Acids Research 128711-8721.
Borghi (2010) Methods Mol Biol 655, 65-75.
Bortesi and Fischer (2015) Biotechnology Advances 33:41-52.
Bostwick (2012) Mayo Clinical Proceedings 87:172-186.
Boynton et al. (1993) Methods Enzymology 217:510-36.
Brown (editor) (1991) Essential Molecular Biology: A Practical Approach, Volumes 1 and 2, IRL Press.
Bruce (2000) Trends in Cell Biology 10:440-447.
Busk et al. (1997) Plant J. 11:1285-95.
Cadwell and Joyce (1992) Genome Research 2:28-33.
Carrier et al. (2006) Proceedings of the National Academy of Sciences of the USA. 103:7895-7900.
Carrington and Freed (1990) J Virol 64:1590-7.
Cazzonelli et al. (2005) Transgenic Research 14, 941-967.
Chandler et al. (1989) Plant Cell (1): 1175-1183.
Citti et al. (2019) Scientific Reports 9:20335.
Colgrave et al. (2019) Food Chem Toxicol. 130:89-98.
Christou et al. (1991) Nature Biotechnology 9:957-962.
Coco (2002).
Coligan et al. (editors) Current Protocols in Immunology, John Wiley & Sons (including all updates until present).
Corrado and Karali (2009) Biotechnology Advances 27, 733-743.
Coutu et al. (2007) Transgenic Research 16:771-781.
Crameri et al. (1998) Nature 391: 288-91.
Deguchi et al. (2020) Frontiers in Plant Science 11:580621.
Dellaporta et al. (1988) Chromosome Structure and Function 263-282.
Depicker et al. (1982) Journal of Molecular and Applied Genetics 1:561-573.
Diamos and Mason (2019) Front Plant Sci. 9:1974.
Doudna and Charpentier (2014) Science 346.
Dugdale et al. (2013) The Plant Cell. 25:2429-2443.
Dugdale et al. (2014) Nature Protocols 9:1010-1027.
Ebert et al. (1987) Proceedings of the National Academy of Sciences of the USA 84(16): 5745-5749.
Eggert et al. (2005) Chembiochem 6:1062-1067.
Engler et al. (2014) ACS Synthetic Biology 3:839.
Fan et al. (2015) Scientific Reports. 5:12217.
Fellermeier et al. (1998) FEBS Letters 427:283-285.
Fellermeier et al. (2001) European Journal of Biochemistry 268:1596-1604.
Freytag et al. (1988) Plant Cell Reports 7:30-34.
Gagne et al. (2012) Proceedings of the National Academy of Sciences of the USA 109:12811-128106.
Geissler et al. (2018) Biotechnology Letters 40:981-987.
Glick and Thompson, (eds.), Methods in Plant Molecular Biology and Biotechnology, Boca Raton, Fla.: CRC Press (1993).
Glover and Hames (editors), (1995 and 1996) DNA Cloning: A Practical Approach, Volumes 1-4, IRL Press.
Guan and Nikolau (2016) Plant J. 85:581-93.
Gülck and Moller (2020) Trends in Plant Science 52:985-1004.
Gülck et al. (2020) Journal of Natural Products 83:2877-2893.
Golds et al. (1993) Nature Biotechnology 11:95-97.
Gordon-Kamm et al. (1990) The Plant Cell 2:603-618.
Haft et al. (2005) PLOS Computational Biology 1:e60.
Hanley-Bowdoin (1986) In vitro synthesis of chloroplast messenger RNAs. PhD Dissertation. The Rockefeller University, NY.
Hanley-Bowdoin and Chua, (1987) Trends in Biomedical Sciences 12:67-70.
Hanson et al (2012).
Harayama (1998) Trends in Biotechnology 16:76-82.
Hazekamp (2018) Medical Cannabis and Cannabinoids. 1:65-72.
Hazekamp et al. (2004) Journal of Liquid Chromatography & Related Technologies 27:2421-2439.
Hellinga (1997) Folding and Design 3: R1-R8.
Hemmerlin et al. (2012) Progress in Lipid Research 51:95-148.
Ikatu et al. (1990) Biotechnology 8(3): 241-242.
Ishida et al. (1996) Nature Biotechnology 14: 745-750.
Jarvis (2008) New Phytologist 179:257-285.
Jefferson et al. (1987) The EMBO Journal 6:3901-3907.
Jézéquek et al. (2008).
Jinek et al. (2012) Science 337:816-821.
Jones et al. (2010) Journal of Pharmacology and Experimental Therapeutics 332:569-577.
Kalinger et al. (2021) Lipids 56:327-344.
Katz et al. (1983) The Journal of General Microbiology 129: 2703-2714.
Kearsey et al. (2020) FEBS Journal 287:1511-1524.
King (2019) Current Opinion in Food Science 28:32-40.
Kinghorn et al. (2017) Phytocannabinoids, Springer, Switzerland.
Klee et al. (1985) Nature Biotechnology 3:637-642.
Klein et al. (1987) Nature 327:70-73.
Knoblauch et al. (1999) Nature Biotechnology 17, 906-909.
Kohler et al. (1996) The Plant Journal 10, 175-183.
Krebbers et al. (1982) Nucleic Acid Research 10:4985-5002.
Lange et al. (2016) Journal of Biotechnology 233:42-48.
Lawton et al. (1987) Molecular and Cellular Biology 7:335-341.
Lee et al. (2002) Molecular and Cells 14:388-397.
Lee et al. (2008) The Plant Cell. 20:1603-1622.
Leung et al. (1989) Science 246:1306-1309.
Li et al. (1996) FEBA Lett 379:117-21.
Liang et al. (2017) Nature Communications 18:14261.
Liang et al. (2018) Plant Biotechnology Journal 16: 2053-2062.
Liang et al. (2019) Methods in Molecular Biology 1917: 327-335.
Linciano et al. (2020) Scientific Reports 10:2019.
Liu et al. (1998) Journal of General Virology 79:2265-2274.
Lu and Berry (2007) Handbook of Proteins 2, 1153-1157.
Luo et al. (2016) Nucleic Acids Research 44: 7385-7394.
Luo et al. (2019) Nature 567:123.
Ma et al. (2021) BioRxiv 1-21.
Ma et al. (2015) Molecular Plant 8:1274-1284.
Makarova et al. (2015) Methods in Molecular Biology 1311: 47-75.
Matsuoka et al. (1994) Plant J 6:311-319.
McBride et al. (1994) Proceedings of the National Academy of Sciences of the USA 91: 7301-7305.
Meier et al. (1997) FEBS Lett 415:91-5.
Mullet et al. (1985) Plant Molecular Biology 4:39-54.
Muthan et al. (2013) Plant Physiology 163:61-74.
Needleman and Wunsch (1970) J Mol Biol. 48:443-453.
Ness et al. (2002) Nature Biotechnology 20: 1251-1255.
Noh and Amasino (1999) Plant Molecular Biology 41: 181-194.
Odell et al. (1985) Nature 313:810-812.
O'Neill et al. (1993) The Plant Journal 3: 729-738.
Ostermeier et al. (1999) Nature Biotechnology 17:1205-1209.
Ow et al. (1986) Science 234:856-859.
Ozber et al. (2020) Journal of Industrial Microbiology & Biotechnology 47:815-828.
Perbal (1984) John Wiley and Sons.
Rasala et al. (2011) Plant Biotechnology Journal 9:674-683.
Reid (2018) American Chemical Society: pp I+EC-160.
Rios et al. (2002) The Plant Journal 32:243-253.
Russo (2016) Trends in Pharmacological Sciences 37:594-605.
Sambrook et al. (1989) Cold Spring Harbour Laboratory Press.
Schachtsiek et al. (2018) Planta Medica 84:214-220.
Schena et al. (1991) Proc. Natl. Acad. Sci. USA 88:0421.
Sfecci et al. (2017) Phytochemistry Letters. 20:406-409.
Shaner et al. (2013) Nature Methods 10:407-409.
Shen et al. (2014) Journal of Experimental Botany 65:419-428.
Shiina et al. (1997) Plant Physiol. 115:477-83.
Shockey and Browse (2011) The Plant Journal. 66:143-160.
Shoyama et al. (1975) Phytochemistry 14:2189-2192.
Sieber et al. (2001) Nature Biotechnology 19: 456-460.
Sirikantaramas et al. (2004) Journal of Biological Chemistry 279:39757-39774.
Sirikantaramas et al. (2005) Plant Cell Physiology 46:1278-1582.
Sirikantaramas et al. (2017) In: Chandra S., Lata H., ElSohly M. (eds) Cannabis sativa L-Botany and Biotechnology. Springer 183-206.
Śledziński et al. (2021) International Journal of Molecular Sciences 22:263.
Staub et al. (2000) Nature Biotechnology 18:333-338.
Stemmer (1994a) Proceedings of the National Academy of Sciences of the USA 91: 10747-10751.
Stemmer (1994b) Nature 370: 389-391.
Stoddard and Rolland (2019) Plant Direct 3: e00112.
Stout et al. (2012) The Plant Journal 71:353-365.
Sun et al. (2016) Molecular Plant 9: 628-631.
Sutcliffe et al. (1978) Proceedings of the National Academy of Sciences of the USA 75: 3737-3741.
Svab et al. (1990) PNAS 87:8526-8530.
Svab and Maliga (1993) Proceedings of the National Academy of Sciences of the USA 90:913-917.
Svitashev et al. (2016) Nature Communications 7:1-7.
Tan et al. (2018) ACS Synth Biol 17:1886-1896.
Taura et al. (2007) FEBS Letters 581:2929-2934.
Taura et al. (2009) FEBS Letters 583:2061-2066.
Thomas et al. (2020) Applied Microbiology and Biotechnology 104: 9551-9563.
Turner and Foster (1995) Molecular Biotechnology 3:225-236.
Valliere et al. (2019) Nature Communications 10:565.
Valliere et al. (2020) Nature Chemical Biology 16:1427.
Vanhercke et al. (2014) Plant Biotechnology Journal 12:231-239.
Vanhercke et al. (2017) Metabolic Engineering 39:237-246.
Vasil et al. (1993) Nature Biotechnology 11:1553-1558.
Verma and Daniell (2007) Plant physiology 145:1129-1143.
Volkov et al. (1999) Molecular Biology Evolution 16: 311-320.
Volkow et al. (2014) The New England Journal of Medicine 370:2219-2227.
Wan and Lemeaux (1994) Plant Physiology 104:37-48.
Wang et al. (1995) Plant Molecular Biology 28: 619-634.
Wang et al. (2017) The Plant Cell 29:1678-1696.
Ward et al. (1993) Plant Mol. Biol. 22:361-366.
Weber et al. (2011) PLoS One 6:e16756.
Weeks et al. (1993) Plant Physiology 102:1077-1084.
Weissbach and Weissbach (1989) Methods for Plant Molecular Biology Academic Press.
Wojcik et al. (2021) Plant Physiology 185:608-618.
Woo et al. (2015) Nature Biotechnology 33:1162-1164.
Wood et al. (2009) Plant Biotechnology Journal 7:914-924.
Yang and Russell (1990) PNAS 87:4144-4148.
Zhang and Masson (2006) Biotechnology and Bioengineering 93:271-279.
Zhao et al. (1998) Nature Biotechnology 16: 258-261.
Zirpel et al. (2015) Biotechnology Letters 37:1869-1875.
Zirpel et al. (2018) Journal of Biotechnology 284:17-26.
Zukowski et al. (1983) PNAS 80:1101-1105.
Zurawski et al. (1981) Nucleic Acids Research 9:3251-3270.
Zurawski et al. (1982) PNAS 79:7699-7703.

1. A plastid of a genetically modified plant or part thereof comprising an acyl activating enzyme 1 (AAE1) polypeptide, an olivetol synthase (OLS) polypeptide and an olivetolic acid cyclase (OAC) polypeptide, optionally further comprising a plastid localised prenyltransferase polypeptide, preferably cannabigerolic acid synthase (CBGAS),
wherein the polypeptides increase the production of cannabigerolic acid (CBGA) in the presence of hexanoic acid (C6) and geranyl-pyrophosphate (GPP), when compared to a plastid of a wild-type plant or part thereof.

2. A genetically modified plant or part thereof comprising a polynucleotide encoding an acyl activating enzyme 1 (AAE1), a polynucleotide encoding an olivetol synthase (OLS) and a polynucleotide encoding an olivetolic acid cyclase (OAC), optionally further comprising a polynucleotide encoding a plastid localised prenyltransferase polypeptide, preferably cannabigerolic acid synthase (CBGAS),
wherein each polynucleotide is operably linked to a promoter which is capable of directing expression of the polynucleotide in the plant or part thereof,
wherein the polynucleotides are codon optimised for expression in the plant or part thereof, and
when expressed in the plant or part thereof in the presence of hexanoic acid (C6) and geranyl-pyrophosphate (GPP), the polypeptides encoded by the polynucleotides increase the production of cannabigerolic acid (CBGA) when compared to a wild-type plant or part thereof.

3. The genetically modified plant or part thereof of claim 2,
wherein
i) the polynucleotides are comprised within a vector, preferably a viral vector,
wherein the polynucleotides encode a fusion polypeptide comprising a plastid transporting peptide; and/or,
wherein the polypeptide encoded by the polynucleotides are expressed in the presence of a plastid localised prenyltransferase; and/or
ii) when expressed in the plant or part thereof in the presence of hexanoic acid (C6) and geranyl-pyrophosphate (GPP), the polypeptides encoded by the polynucleotides increase the production of cannabigerolic acid (CBGA) compared to a wild-type plant or part thereof.

4. (canceled)

5. The plant or part thereof of claim 2, wherein the genetically modified plant or part thereof is a high biomass plant, preferably selected from the group consisting of Acrocomia aculeata (macauba palm), Arabidopsis thaliana, Aracinis hypogaea (peanut), Astrocaryum murumuru (murumuru), Astrocaryum vulgare (tucuma), Attalea geraensis (Indaia-rateiro), Attalea humilis (American oil palm), Attalea oleifera (andaia), Attalea phalerata (uricuri), Attalea speciosa (babassu), Avena sativa (oats), Beta vulgaris (sugar beet), Brassica sp. such as Brassica carinata, Brassica juncea, Brassica napobrassica, Brassica napus (canola), Camelina sativa (false flax), Cannabis sativa (hemp), Carthamus tinctorius (safflower), Caryocar brasiliense (pequi), Cocos nucifera (Coconut), Crambe abyssinica (Abyssinian kale), Cucumis melo (melon), Elaeis guineensis (African palm), Glycine max (soybean), Gossypium hirsutum (cotton), Helianthus sp. such as Helianthus annuus (sunflower), Hordeum vulgare (barley), Jatropha curcas (physic nut), Joannesia princeps (arara nut-tree), Lemna sp. (duckweed) such as Lemna aequinoctialis, Lemna disperma, Lemna ecuadoriensis, Lemna gibba (swollen duckweed), Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Licania rigida (oiticica), Linum usitatissimum (flax), Lupinus angustifolius (lupin), Mauritia flexuosa (buriti palm), Maximiliana maripa (inaja palm), Miscanthus sp. such as Miscanthus x giganteus and Miscanthus sinensis, Nicotiana sp. (tobacco) such as Nicotiana tabacum, Nicotiana benthamiana, Oenocarpus bacaba (bacaba-do-azeite), Oenocarpus bataua (pataua), Oenocarpus distichus (bacaba-de-leque), Oryza sp. (rice) such as Oryza sativa and Oryza glaberrima, Panicum virgatum (switchgrass), Paraqueiba paraensis (mari), Persea amencana (avocado), Pongamia pinnata (Indian beech), Populus trichocarpa, Ricinus communis (castor), Saccharum sp. (sugarcane), Sesamum indicum (sesame), Solanum tuberosum (potato), Sorghum sp. such as Sorghum bicolor, Sorghum vulgare, Theobroma grandiforum (cupuassu), Trifolium sp., Trithrinax brasiliensis (Brazilian needle palm), Triticum sp. (wheat) such as Triticum aestivum and Zea mays (corn).

6. The plant or part thereof of claim 5, wherein the plant is Nicotiana sp., preferably Nicotiana benthamiana or Nicotiana tabacum.

7-8. (canceled)

9. The plant or part thereof of claim 2, wherein at least one polypeptide is active in the cytosol of the plant or part thereof; and/or
wherein one or more polynucleotides, optionally each polynucleotide, encode a fusion polypeptide comprising a plastid transporting peptide, preferably a chloroplast transit peptide (CTP).

10-23. (canceled)

24. The plant or part thereof of claim 2, wherein the plant or part thereof further comprises at least one polynucleotide selected from the group consisting of a polynucleotide encoding an acyl-lipid thioesterase, optionally wherein the polynucleotide encodes a fusion polypeptide comprising a plastid transporting peptide.

25. (canceled)

26. A nucleic acid construct encoding polypeptides for expression in a plant or part thereof, comprising one or more polynucleotides encoding an acyl activating enzyme 1 (AAE1), a polynucleotide encoding an olivetol synthase (OLS), and a polynucleotide encoding an olivetolic acid cyclase (OAC), optionally further encoding one or more of: a plastid transporting peptide, a prenyltransferase, a plastid acyl-lipid thioesterase, a plastid lipase, and a plastid tomato 13-lipoxygenase;
wherein the polynucleotide is operably linked to a promoter which is capable of directing expression of the one or more or all polynucleotides in the plant or part thereof,
optionally, wherein the one or more or all polynucleotides are operably linked to a polynucleotide encoding a plastid transporting peptide;
optionally, wherein the nucleic acid construct encodes a fusion polypeptide comprising the plastid transporting peptide, and
when expressed, the polypeptide encoded by the polynucleotide/s increase the production of cannabigerolic acid (CBGA) compared to a wild-type plant or part thereof.

27. (canceled)

28. The nucleic acid construct of claim 26, further comprising one or more of the following features:
i) a polynucleotide encoding a silencing suppressor polypeptide, preferably a p19 silencing suppressor polypeptide;
ii) the nucleic acid construct is comprised within a vector suitable for expression in a plant or part thereof;
iii) one or more or all polynucleotides are operably linked to a promoter which is capable of directing expression of the polypeptide encoded by the polynucleotide in a plant or part thereof;
iv) the one or more or all polynucleotides are operably linked to a polynucleotide encoding a plastid transporting peptide;
v) the nucleic acid construct encodes a fusion polypeptide comprising a plastid transporting peptide;
vi) the acyl-lipid thioesterase is ALT4;
vii) the plastid lipase is plastid lipase 1 (PLIP1);
viii) production of CBGA is increased by at least about 1.5-about 2 fold, about 2-about 2.5 fold, about 2.5-about 3.0 fold, about 3-about 3.5 fold, about 3.5-about 4.0 fold, about 4-about 4.5 fold, about 4.5-about 5.0 fold or about 5-about 5.5 fold or more when expressed in a plant or part thereof, compared to a wild-type plant or part thereof; and
ix) production of CBGA is increased at least about 10%-about 20%, about 20%-about 30%, about 30%-about 40%, about 40%-about 50%, about 50%-about 60%, about 60%-70%, about 70%-about 80%, about 80%-about 90%, about 90%-about 100%, about 100%-about 120%, about 120%-about 140%, about 140%-about 160%, about 160%-about 180%, about 180%-about 200%, about 200%-about 220%, about 220%-about 240%, about 240%-about 260%, about 260%-about 280%, about 280%-about 300%, about 300%-about 320%, about 320%-about 340%, about 340%-about 360%, about 360%-about 380%, about 400%-about 420% or more when expressed in a plant or part thereof, compared to a wild-type plant or part thereof.

29-32. (canceled)

33. A fusion polypeptide for expression in a plant or part thereof, comprising:
i) a polypeptide selected from the group consisting of a polyketide synthase, a polyketide cyclase, an acyl-activating enzyme, a prenyltransferase, a plastid acyl-lipid thioesterase, a plastid lipase or a plastid tomato 13-lipoxygenase, and
ii) a plastid transporting sequence,
wherein the polypeptide of (i) increases the production of cannabigerolic acid (CBGA) in the presence of hexanoic acid (C6) and geranyl-pyrophosphate (GPP), when compared to a wild type plant or part thereof, optionally wherein the plastid transporting sequence is a chloroplast transit peptide (CTP).

34-36. (canceled)

37. The nucleic acid construct of claim 26, wherein the polyketide synthase is olivetol synthase (OLS), the polyketide cyclase is olivetolic acid cyclase (OAC), the acyl-activating enzyme is acyl activating enzyme 1 (AAE1), the plastid acyl-lipid thioesterase is ALT4, the plastid lipase is PLIP1, the plastid tomato 13-lipoxygenase is TomLoxC and the prenyltransferase is cannabigerolic acid synthase (CBGAS), preferably wherein plastid transporting peptide is a chloroplast transit peptide (CTP).

38. The the plant or part thereof of claim 2, wherein one or more apply:
i) the polynucleotide encoding AAE1 comprises a nucleotide sequence which is at least 70%, at least 75%, at least 80%, at least 85%, at least 90%, at least 95%, at least 96%, at least 97%, at least 98%, at least 99%, at least 99.5% at least 99.8% or identical to a sequence set forth as any one of SEQ ID NO:5, SEQ ID NO:6 and SEQ ID NO:7;
ii) the polynucleotide encoding OLS comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to a sequence set forth as any one of SEQ ID NO:8, SEQ ID NO:9 and SEQ ID NO:10;
iii) the polynucleotide encoding OAC comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to a sequence set forth as any one of SEQ ID NO:11, SEQ ID NO:12 and SEQ ID NO:13;
iv) the polynucleotide encoding the plastid transporting peptide comprises a sequence which is at least 70%, at least 75%, at least 80%, at least 85%, at least 90%, at least 95%, at least 96%, at least 97%, at least 98%, at least 99%, at least 99.5%, at least 99.8% or identical to the sequence set forth as SEQ ID NO:15.

39. (canceled)

40. The plant or part thereof of claim 24, wherein:
i) the polynucleotide encoding ALT4 comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to a sequence set forth as SEQ ID NO:24 or 25;
ii) the polynucleotide encoding PLIP1 comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to the sequence set forth as SEQ ID NO:26; and
iii) the polynucleotide encoding TomLoxC comprises a nucleotide sequence which is at least 70%, at least 75%, at least 80%, at least 85%, at least 90%, at least 95%, at least 96%, at least 97%, at least 98%, at least 99%, at least 99.5%, at least 99.8% or identical to the sequence set forth as SEQ ID NO:27.

41-49. (canceled)

50. A method of producing a cannabinoid in a plant or plant part thereof, the method comprising:
i) obtaining a plant or part thereof comprising the nucleic acid construct of claim 26 cultivated under conditions sufficient for expression of the polypeptide in the plant or plant part, wherein when expressed in the presence of hexanoic acid (C6) and geranyl-pyrophosphate (GPP), the polypeptide increases the production of cannabigerolic acid (CBGA) in the plant or plant part when compared to a wild type plant or plant part;
ii) recovering the CBGA produced at i); and
iii) optionally feeding the CBGA to a genetically modified microorganism, preferably yeast or bacteria; and
iv) chemically, physically, or biochemically converting the CBGA, optionally by (a) using the CBGA as a feedstock; or optionally by (b) CBGA decarboxylation, preferably by incubation of CBGA with heat or light;
thereby producing the cannabinoid.

51-63. (canceled)

64. A genetically modified and/or recombinant plant cell for producing cannabigerolic acid (CBGA) comprising the nucleic acid construct of claim 26.

65-75. (canceled)

76. A process for identifying a polynucleotide encoding a polypeptide for producing cannabigerolic acid (CBGA) in a plant, or part thereof, or plastid thereof, or cell thereof, the process comprising:
i) obtaining the nucleic acid construct of claim 26;
ii) introducing the nucleic acid construct into a plant, part thereof, or plastid or cell thereof;
iii) determining whether the level of CBGA is increased relative to a corresponding wild-type plant, part thereof or plastid or cell thereof lacking the nucleic acid construct; and
iv) optionally, selecting a polynucleotide, which when expressed produces a polypeptide for producing CBGA.

77. (canceled)

78. A process for producing an industrial product, the process comprising the steps of:
i) obtaining a plant or part thereof of claim 2;
ii) processing the plant or part thereof;
iii) converting at least some of a cannabinoid in the plant or part thereof of step i), or in the processed plant, part thereof, or plastid, obtained by step ii), by applying heat, chemical, or enzymatic means, or any combination thereof, to generate a cannabinoid in situ; and
iv) recovering the cannabinoid, thereby producing the industrial product.

79. (canceled)

80. A process for producing extracted cannabigerolic acid (CBGA), the process comprising the steps of:
i) obtaining a plant or part thereof of claim 2;
ii) extracting CBGA from the plant or part thereof; and
iii) recovering the extracted CBGA, thereby producing extracted CBGA.

81-82. (canceled)

83. A process of producing a cannabigerolic acid (CBGA) enriched extract, the process comprising the steps of:
i) obtaining a plant or part thereof of claim 2;
ii) processing the plant or part thereof of step i);
iii) centrifuging the processed plant or part thereof from step ii) to obtain extracts;
iv) filtering the extract obtained by step iii);
v) drying the filtered extract of step iv) and resuspending in buffer solution;
vi) purifying the extract of step v) using a solid-phase extraction column; and
vii) recovering the CBGA enriched extract, thereby producing the CBGA enriched extract.

84. A method for producing a pharmaceutical composition comprising:
i) obtaining the extract of claim 80; and
ii) formulating the extract or cannabinoid with one or more pharmaceutically acceptable carriers, thereby producing a pharmaceutical composition.

85. (canceled)

86. A method of treating and/or preventing a condition or disease responsive to cannabinoid treatment in a subject comprising administering a therapeutically effective amount of the extract of claim 80 to the subject, thereby treating and/or preventing a condition or disease responsive to cannabinoid treatment.

87-89. (canceled)

90. The plant or part thereof of claim 2, wherein the plant or part thereof further comprises at least one polynucleotide selected from a polynucleotide encoding a plastid lipase 1 (PLIP1) and a polynucleotide encoding a tomato 13-lipoxygenase (TomLoxC), optionally wherein the polynucleotide encodes a fusion polypeptide comprising a plastid transporting peptide.
